(19) United States
(12) Patent Application Publication
(10) Pub. No.: US 2016/0215042 A1
(43) Pub. Date: Jul. 28, 2016

(54) METHODS FOR MODULATING IMMUNE RESPONSES DURING CHRONIC IMMUNE CONDITIONS BY TARGETING METALLOTHIONEINS

(71) Applicant: THE BRIGHAM AND WOMEN'S HOSPITAL, INC., Boston, MA (US)

(72) Inventors: Vijay K. KUCHROO, Newton, MA (US); Chuan WU, Boston, MA (US); Caroline POT, Geneva (CH); Lionel APETOH, Dijon (FR); Ana C. ANDERSON, Brookline, MA (US)

(21) Appl. No.: 14/784,721

(22) PCT Filed: Apr. 18, 2014
(86) PCT No.: PCT/US14/34603

§ 371 Date: Oct. 15, 2015

Related U.S. Application Data
(60) Provisional application No. 61/814,059, filed on Apr. 19, 2013.

(51) Int. Cl. C07K 16/18; C12N 15/113

(52) U.S. Cl. CPC C07K 16/18; C12N 15/113; C12N 2310/14; C12N 2310/16; C12N 2310/17; C07K 2317/75; C07K 2317/76

(57) ABSTRACT

Described herein are novel compositions comprising MT1 and/or MT2 modulators (i.e., inhibitors or activators), and methods using these agents for targeting different T-cell populations, for example, type 1 regulatory (Tr1) CD4+ cells and exhausted CD8+ T-cells cells. Aspects of the invention relate to inhibitors of MT1 and/or MT2 to increase the differentiation of CD4+ cell Trl1 CD4+ cells and increase in the activity of Tr1 cells, e.g., to increase IL-10 production. In alternative embodiments, MT1 and/MT1 inhibitors can be used to increase proliferation and/or activity of exhausted CD8+ T-cells and to decrease CD8+ T-cell exhaustion (e.g., decrease functionally exhausted or unresponsive CD8+ immune cells). Accordingly, aspects of the present invention relate to compositions and methods comprising inhibitors of MT1 and/or MT2 useful in the treatment of chronic immune conditions, such as persistent infections, cancer, and autoimmune disease.

### GOVERNMENT SUPPORT

This invention was made with Government Support under Grant Nos. NS038037 and NS076410 awarded by the National Institutes of Health. The Government has certain rights in the invention.

### FIELD OF THE INVENTION

The invention relates to compositions and methods for modulating immune responses.

### SEQUENCE LISTING

The instant application contains a Sequence Listing which has been submitted electronically in ASCII format and is hereby incorporated by reference in its entirety. Said ASCII copy, created on Apr. 17, 2014, is named 043214-077621-PCT_SL.txt and is 38,983 bytes in size.

### BACKGROUND

IL-10-producing type 1 regulatory (Tr1) cells are emerging to be a very important regulatory T cell subset, as reported in Apetoh L, et al. (2010); Nat Immunol 11(9):854-861 and Fitzgerald D C, et al. (2007) Nat Immunol 8(12):1372-1379, which show Tr1 cells are induced by IL-27. Tr1 cells have been reported to control autoimmunity and tissue inflammation in mouse models of human autoimmune diseases including multiple sclerosis, inflammatory bowel disease and graft-versus-host disease (3). In addition, Tr1 cells were reported to suppress the induction of cytotoxic T lymphocyte (CTL) responses and inhibit antitumor immunity (4). Tr1 cells produce both IFN-γ and IL-10, without expressing the regulatory T cell (Treg) specific transcription factor, Foxp3 (5). Transcriptional analysis of Tr1 cells showed that Tr1 cells differentiated from Tbx21^{-/-} cells exhibited severely compromised IFN-γ but not IL-10 production, which was in contrast to Stat1^{-/-} Tr1 cells, which showed reduction in both IFN-γ and IL-10 production (6). More recently, the inventors have identified transcription factors c-Maf and aryl hydrocarbon receptor (AhR), both of which are induced by IL-27, that bind to Il10 promoter and are essential for the induction of IL-10 in Tr1 cells (1, 7, 8). In addition, IL-27 induced Maf and AhR cooperatively bind to the promoter and transactivate Il21 gene, which acts as a growth factor for the generation of Tr1 cells. While molecular landscape for the generation of Tr1 is being identified, very little is known about the molecular switches that can inhibit the development of Tr1 cells.

### SUMMARY OF THE INVENTION

The compositions and methods described herein are based, in part, on the novel discovery that metallothioneins (MTs) are negative regulators of IL-10-producing type 1 regulatory (Tr1) CD4+ cells, and that inhibition of MTs can increase IL-10 production from Tr1 cells, and/or promote or increase their differentiation from CD4+ cells and/or increase the proliferation or activity of Tr1 CD4+ cells. One aspect of the present invention relates to methods and compositions comprising inhibitors of MTs to increase IL-10 production from Tr1 CD4+ cells, for example, in the treatment of autoimmune diseases and treatment of graft-versus-hosts disease and to prevent transplant rejection in a transplant recipient subject. In another aspect of the present invention, overexpression or activation of MTs in Tr1 CD4+ cells can be used to decrease Tr1 CD4+ T cell proliferation and/or differentiation from CD4+ T cells, for example in diseases where excess IL-10 is produced, for example, during chronic immune conditions. In such embodiments, overexpression or activation of MTs in Tr1 CD4+ cells can be performed ex vivo, and the cells administered to a subject in need thereof, e.g., a subject where it is desirable to decrease IL-10 production from Tr1 CD4+ cells.

Furthermore, the inventors have also discovered that MTs regulate different populations of T-cells in different ways. For example, the inventors have discovered that inhibitors of MT1 and/or MT2 can increase exhausted or dysfunctional CD8+ T-cell proliferation and/or activity and can also decrease CD8+ T-cell exhaustion. Accordingly, another aspect of the present invention relates to methods and compositions comprising inhibitors of MTs to decrease CD8+ T cell exhaustion in a subject in need thereof, or to decrease exhausted CD8+ T cell and/or increase CD8+ T cell activity in a subject in need thereof, such as, for example, a subject with a chronic immune condition, e.g., a subject with a chronic infection or cancer.

To identify candidate molecules that can control Tr1 CD4+ cell differentiation, the inventors have performed a comparative gene microarray analysis of Tr1 cells generated with IL-27 and identified that the isoforms 1 and 2 of metallothioneins (MTs) were strongly induced in Tr1 cells by IL-27. MT1 and MT2 are low-molecular weight proteins involved in the detoxification of heavy metals and in the regulation of oxidative stress (9). In mice, there are four different MT genes constitutively expressed in the liver, of which MT1 and MT2 are the most abundantly expressed (10). MT genes are highly induced under different stresses such as inflammation (10) and are specifically induced by proinflammatory cytokines like TNF-α, IL-1 and IL-6 (11). However, the role of MTs in IL-27-induced Tr1 CD4+ differentiation and IL-10 production is novel.

Here, the inventors have discovered that MTs control IL-10 production from Tr1 cells as T cells from MT deficient mice exhibit increased IL-10 production from Tr1 cells both in vitro and in vivo. At the mechanistic level, the inventors discovered that in the absence of MTs or by inhibition of MTs, IL-27 induces increased phosphorylation of STAT1 and STAT3 but not STAT4 in Tr1 CD4+ cells, resulting in enhanced IL-10 production from Tr1 CD4+ cells. Furthermore, compared to wildtype (WT) Tr1 CD4+ cells, Mt^{-/-} Tr1 cells were more efficient in their ability to suppress effector CD8+ T cell proliferation and inhibit development of an autoimmune disease in the Experimental Autoimmune Encephalomyelitis (EAE) model. Taken together, the inventors have discovered that MTs are negative regulators for Tr1 CD4+ cell differentiation and that MTs are new targets for regulating development of Tr1 CD4+ cells.

Furthermore, the inventors have demonstrated that when Tr1 CD4+ cells treated ex vivo with MT activators, e.g., exogenous MT protein, or overexpression of MT protein, are transplanted into an animal model of EAE, the Tr1 cells no longer have the ability to produce IL-10 and are unable to suppress autoimmunity and/or protect the animal from the EAE disease. Thus, activators of MTs can decrease the proliferation or activity of Tr1 CD4+ cells and/or decrease their differentiation from CD4+ cells, thus reducing the production of IL-10 from Tr1 CD4+ cells.

Further, as demonstrated herein, the inventors demonstrate MT1 and/or MT2 are upregulated or increased in dysfunctional or exhausted CD8+ T cells in cancer. MT deficient (Mt^{-/-}) mice exhibit significantly delayed melanoma tumor growth and thus, MT deficiency inhibits the tumor growth; this is accompanied by a failure to generate exhausted CD8+ T cells. Accordingly, the data provided herein identify MTs, in particular MT1 and MT2, as critical inducers of IL-27-mediated CD8+ T cell exhaustion/dysfunction during chronic immune conditions, e.g., chronic infections and cancer. Accordingly, one aspect of the present invention relates to methods and compositions comprising MT inhibitors to decrease the proliferation and/or differentiation of CD8+ T-cells, and to decrease CD8+ T-cell exhaustion, for example, for the treatment of chronic immune conditions, e.g., chronic infections and cancer.

One aspect of the present invention relates to a method for promoting CD4+ T cell differentiation into type 1 regulatory T (Tr1) CD4+ cells and/or activity of type 1 regulatory T (Tr1) CD4+ cells in a subject in need thereof, comprising administering to the subject a composition comprising an inhibitor of a metallothionein (MT), referred to as a “MT inhibitor” as disclosed herein. Another aspect of the present invention relates to a method to increase IL-10 secretion from type 1 regulatory T (Tr1) CD4+ cells in a subject in need thereof, comprising administering to the subject a composition comprising an inhibitor of a metallothionein (MT).

Another aspect of the present invention relates to a pharmaceutical composition comprising an inhibitor of a metallothionein (MT) and a pharmaceutically acceptable carrier to promote the differentiation of CD4+ cells to type 1 regulatory T (Tr1) CD4+ cells in a subject in need thereof. Another aspect of the present invention relates to a pharmaceutical composition comprising an inhibitor of a metallothionein (MT) and a pharmaceutically acceptable carrier to increase IL-10 production from type 1 regulatory T (Tr1) CD4+ cells in a subject in need thereof.

In all aspects herein, in some embodiments, a MT inhibitor for use in the methods and compositions as disclosed herein inhibits an isoform of metallothionein 1 (MT1), and/or metallothionein 2 (MT2). In some embodiments, the MT inhibitor inhibits MT1e and/or MT1h.

In all aspects of the present invention herein, in some embodiments, a MT inhibitor is selected from the group consisting of: RNAi agent, oligonucleotide, antibody, antibody fragment, peptide inhibitor, protein inhibitor, aptamer, and functional fragments thereof.

In some embodiments, where an MT inhibitor is used promote the differentiation of T-cells to CD4+ cells, and/or increase the activity of Tr1 cells in a subject in need thereof, the subject in need thereof has an autoimmune disease and/or is a transplant recipient. In some embodiments, the subject in need thereof has an autoimmune disease which is myelitis, for example, but not limited to poliomyelitis (PM), dermatomyositis (DM) or inclusion body myositis (IBM). In some embodiments, the subject in need thereof has an autoimmune disease selected from the group consisting of: Addison's disease, Celiac disease (gluten-sensitive enteropathy), Graves disease, Hashimoto's thyroiditis, Multiple sclerosis, Myasthenia gravis, Pernicious anemia; Reactive arthritis, Rheumatoid arthritis, Sjogren's syndrome, Systemic lupus erythematosus, inflammatory bowel disease (IBS), graft-versus-host disease and Type I diabetes.

In some embodiments, the increased activity of the Tr1 cells is increased activity of IL-27-induced Tr1 CD4+ cells. In some embodiments of this aspect of invention, a MT inhibitor can be used to treat Tr1 CD4+ cells ex vivo, and where the treated Tr1 CD4+ cells are administered to a subject in need thereof, e.g., a subject with an autoimmune disease and/or a transplant recipient.

Another aspect of the present invention relates to a method for increasing the differentiation and/or proliferation of functionally exhausted CD8+ T-cells in a subject in need thereof, comprising administering to the subject a composition comprising an inhibitor of a metallothionein (MT). Another aspect of the present invention relates to a method for decreasing CD8+ T-cell exhaustion in a subject in need thereof, comprising administering to a subject an effective amount of a pharmaceutical composition comprising an inhibitor of a metallothionein (MT).

Another aspect of the present invention relates to a pharmaceutical composition comprising an inhibitor of a metallothionein (MT) and a pharmaceutically acceptable carrier to increase the activity and/or proliferation of exhausted CD8+ T-cells in a subject in need thereof. Another aspect of the present invention relates to a pharmaceutical composition comprising an inhibitor of a metallothionein (MT) and a pharmaceutically acceptable carrier to decrease CD8+ T-cell exhaustion in a subject in need thereof.

In some embodiments, a MT inhibitor for use in the methods and compositions to increase the proliferation and/or differentiation of functionally exhausted CD8+ T cells are used to treat a subject with cancer, and/or a chronic immune disease, e.g., a chronic infection. In some embodiments, the is metastatic cancer, for example, but not limited to, metastatic cancer selected from the group consisting of: colon cancer, lung cancer, prostate cancer, breast cancer, kidney cancer, leukemia, blood cancer and the like. In some embodiments, a MT inhibitor can be used to treat CD8+ T-cells ex vivo, and where the treated CD8+ T-cells are subsequently administered to a subject in need thereof, e.g., a cancer subject or a subject with a chronic immune disease, e.g., chronic infection.

Another aspect of the present invention relates to a method for promoting the differentiation of CD4+ T-cells to type 1 regulatory T (Tr1) CD4+ cells, and/or activity of type 1 regulatory T (Tr1) CD4+ cells in a subject in need thereof, the method comprising administering to the subject a composition comprising an activator agent which increases the phosphorylation of STAT1 and/or STAT3 or increases the phosphorylation of STAT1 and/or STAT3. Another aspect of the present invention relates to a method to increase IL-10 secretion from type 1 regulatory T (Tr1) CD4+ cells in a subject in need thereof, the method comprising administering to the subject a composition comprising an activator agent which increases the phosphorylation of STAT1 and/or STAT3, or increases the phosphorylation of STAT1 and/or STAT3.

In some embodiments, a subject in need thereof who is administered an activator agent which increases the phosphorylation of STAT1 and/or STAT3 or increases the phosphorylation of STAT1 and/or STAT3 has an autoimmune disease. In some embodiments, the autoimmune disease is myelitis e.g., polymyositis (PM), dermatomyositis (DM) or inclusion body myositis (IBM). In some embodiments, the subject in need thereof has an autoimmune disease selected from the group consisting of: Addison's disease, Celiac disease (gluten-sensitive enteropathy), Dermatomyositis, Graves disease, Hashimoto's thyroiditis, Multiple sclerosis, Myasthenia gravis, Pernicious anemia; Reactive arthritis, Rheumatoid arthritis, Sjogren syndrome, Systemic lupus erythematosus, inflammatory bowel disease (IBS), graft-versus-host disease and Type I diabetes.

Another aspect of the present invention relates to a pharmaceutical composition comprising an activator of a metallothionein (MT) and a pharmaceutically acceptable carrier to decrease the differentiation of CD4+ cells to type 1 regulatory T (Tr1) CD4+ cells and/or activity of type 1 regulatory T (Tr1) CD4+ cells in a subject in need thereof. Another aspect of the present invention relates to a pharmaceutical composition comprising an activator of a metallothionein (MT) and a pharmaceutically acceptable carrier to decrease IL-10 production from type 1 regulatory T (Tr1) CD4+ cells in a subject in need thereof. In some embodiments,

In all aspects herein, a MT activator for use in the methods and compositions as disclosed herein increases the activity or function of an isoform of metallothionein 1 (MT1), and/or metallothionein 2 (MT2). In some embodiments, the MT inhibitor inhibits MT1e and/or MT1h.

In some embodiments, a MT activator for use in the methods and compositions as disclosed herein is selected from the group consisting of: an antibody, antibody fragment, peptide, protein, small molecule, and functional fragments thereof.

In some embodiments, a MT activator for use in the methods and compositions as disclosed herein is administered to a subject in need thereof, for example, a subject who is in need of reduced IL-10 production.

In some embodiments of these methods and all such methods described herein, the MT1 and/or MT2 inhibitor is an anti-MT1 or anti-MT2 antibody or antigen-binding fragment thereof, a small molecule MT1 or MT2 inhibitor, an RNA interference molecule, an RNA or DNA aptamer that binds or physically interacts with and inhibits MT1 and/or MT2, or a dominant negative peptide inhibitor of MT1 and/or MT2.

In some embodiments of these methods and all such methods described herein, a subject being administered a MT1 and/or MT2 inhibitor to increase the differentiation and/or proliferation of Tr1 CD4+ cells and/or increase IL-10 production from Tr1 CD4+ cells is diagnosed as having an autoimmune disease or disorder or is diagnosed as having graft versus host disease or is a transplant recipient.

In some embodiments of these methods and all such methods described herein, the subject being administered a MT1 and/or MT2 inhibitor to decrease the differentiation and/or proliferation of CD8+ T− cells and/or decrease CD8+ T cell exhaustion, is diagnosed as having a chronic immune disease, e.g., a chronic or persistent infection or cancer. In some embodiments, the cancer is metastatic cancer.

### BRIEF DESCRIPTION OF THE FIGURES

FIGS. 1A-1C shows IL-27 induces Mt1 and Mt2 in Tr1 CD4+ cells. RNA was isolated from naïve cells cultured with IL-27 (Tr1 cells, open squares) or without IL-27 (Th0 cells, closed squares) and was subjected to RT-PCR to examine expression levels of Mt1, Mt2 and IL10 at different time points following activation. FIG. 1A shows levels of MT1 in Tr1 CD4+ cells, FIG. 1B shows levels of MT2 in Tr1 CD4+ cells and FIG. 1C shows levels of IL-10 produced from Tr1 CD4+ cells. RT-PCR data are normalized to β-actin mRNA and are representative of three independent experiments.

FIGS. 2A-2F show MTs impair IL10 expression by IL-27-induced Tr1 CD4+ cells. FIG. 2A shows naïve CD4⁺ T-cells from WT or Mt^{-/-} mice differentiated towards Tr1 CD4+ cell phenotype and the frequencies of IL-10 and IFN-γ expressing cells were determined by flow cytometry after 4 days of stimulation. FIG. 2B shows levels of IL-10 and IFN-γ secretion as measured by ELISA at 72 h. FIG. 2C shows the expression levels of Tr1 CD4+ cell signature genes as determined by RT-PCR 72 h after stimulation and are displayed as values normalized to mRNA expression of β-actin. FIG. 2D shows IFN-γ and IL-10 expression as determined by FACS and FIG. 2E shows IFN-γ and IL-10 expression as determined by ELISA in WT Tr1 CD4+ cells transduced with control retrovirus, MT1 or MT2-expressing retrovirus and activated in the presence of IL-27, demonstrating that MT1 or MT2 expression decreases IL-10 production from Tr1 CD4+ cells; FIG. 2F shows 3 d after stimulation, retrovirus-transduced T cells were sorted for GFP⁺ cells and RT-PCR was subsequently performed to examine the expression levels of IL-10, LFNg and AHR, demonstrating that MT1 or MT2 expression decreases IL-10 production from Tr1 CD4+ cells. The data are representative of three independent experiments. *P<0.05 (Student's t-test).

FIGS. 3A-3G show STAT1/3 and MTs form a kinetic balance to control IL-10 production in Tr1 cells. FIG. 3A and FIG. 3B show the levels of intracellular phosphorylated STAT1 (p-STAT1), STAT3 (p-STAT3) or STAT4 (p-STAT4) which were determined 20 min after stimulation of CD4⁺ Tr1 cells from WT or Mt^{-/-} mice (unstimulated or IL-27-treated) by flow cytometry; FIG. 3C shows the quantification of MFI of p-STAT1, p-STAT3 and p-STAT4; FIG. 3D shows the expression levels of indicated genes in differentiated Tr1 cells from WT and Stat1^{-/-} or Stat3^{-/-} mice were determined by RT-PCR; FIG. 3E shows that 24 h after activation, CD4⁺ T cells were transduced with the indicated combinations of retroviruses and subsequently cultured in the presence of IL-27 for 3 d prior to stimulation with PMA and ionomycin for 4 h. Expression levels of IL-10 were determined by intracellular cytokine staining (data shown are gated on GFP⁺ Thy1.1⁺ cells). FIG. 3F shows the quantification of CD4⁺GFP⁺ Thy1.1⁺IL-10⁺ T cells were transduced with the indicated retroviruses as in FIG. 3D; FIG. 3G shows FACS-sorted CD4⁺GFP⁺ Thy1.1⁺ cells were stimulated for 4 h with PMA and ionomycin. Levels of secreted IL-10 in the culture media were measured by ELISA. The data are representative of three independent experiments. *P<0.05 (Student's t-test).

FIGS. 4A-4C shows MTs control IL-10-producing T cells in vivo. FIG. 4A shows memory CD4⁺CD25⁻CD44^{hi}CD62L⁻ cells from WT or Mt^{-/-} mice were reactivated in vitro with anti-CD3 and anti-CD28 and their IL-10 production assessed by ELISA after 4 days of culture. FIG. 4B shows results from WT or Mt^{-/-} chimera mice (n=5) which were injected i.p. with 20 μg anti-CD3 antibody or PBS once every 3 days, for a total of 4 times. 4 hours after the last injection, mice were sacrificed and IL-10 production of CD4⁺ T cells from peyer's patches (PP) and lamina propria (LP) was analyzed by flow cytometry. FIG. 4C shows the quantification of the percentage of CD4⁺IL-10⁺Foxp3⁻IL17⁻ T cells from PP and LP of WT or Mt^{-/-} chimera mice. The data are representative of three independent experiments. *P<0.05 and **P<0.01 (Student's t-test, error bars, SD).

FIGS. 5A-5F show MTs regulate suppressive capacity of Tr1 CD+ cells in vivo. Lymphocytes were isolated from WT and Mt^{-/-} mice 10 days after immunization with MOG₃₅₋₅₅ and were re-stimulated with MOG₃₅₋₅₅ or OVA₃₂₃₋₃₃₉ in combination with IL-23 or IL-27 for 3 days. FIG. 5A shows the frequency of IL-10/IL-17- and IFN-γ-expressing cells as determined by flow cytometry results of WT and Mt^{-/-} lymphocytes stimulated with MOG₃₅₋₅₅ or OVA₃₂₃₋₃₃₉ in combination with IL-23. FIG. 5B shows IL-17 and IFN-γ secretion of WT and Mt^{-/-} lymphocytes stimulated with MOG₃₅₋₅₅ or OVA₃₂₃₋₃₃₉ in combination with IL-23 as measured by ELISA. FIG. 5C shows the frequency of IL-10/IL-17- and IFN-γ-expressing cells as determined by flow cytometry results of WT and Mt^{-/-} lymphocytes stimulated with MOG₃₅₋₅₅ or OVA₃₂₃₋₃₃₉ in combination with IL-27. FIG. 5D shows IL-10 and IFN-γ secretion of WT and Mt^{-/-} lymphocytes stimulated with MOG₃₅₋₅₅ or OVA₃₂₃₋₃₃₉ in combination with IL-27 as measured by ELISA. FIG. 5E shows CD4⁺ T cells which were isolated from lymph nodes of MOG₃₅₋₅₅ immunized WT and Mt^{-/-} mice, and subsequently re-stimulated in vitro with MOG₃₅₋₅₅ and IL-23 (effector cells) or IL-27 (Tr1 cells). Left shows EAE development in WT (129/Sv B6 F1) recipients adoptively transferred with WT MOG₃₅₋₅₅ specific effector T cells with (3:1) or without WT or Mt^{-/-} Tr1 cells. Right shows the data expressed as a linear-regression curve of the disease score over time. FIG. 5F shows an embodiment of the experimental setup as (e), except for labeling WT effector T cells with CFSE prior to adoptive transfer. 4 days after transfer, the percentage of proliferating CFSE-labeled CD4⁺ T cells in the lymph nodes was determined by flow cytometry. The data are representative of three independent experiments. *P<0.05 and **P<0.01 (Student's t-test, error bars, SD).

FIGS. 6A-6B. FIG. 6A shows Mt1, Mt2 and Il10 expression in Tr1 CD4+ cells relative to Th0 cells. Naïve CD4⁺CD44^{lo}CD62L⁺CD25⁻Tr1 cells were differentiated with IL-27 (50 ng/ml) (Tr1) or without cytokines (Th0) in the presence of plate-bound anti-CD3 and anti-CD28 antibodies (1 μg/ml). After 72 hours, RNA was extracted and subjected to Affymetrix array analysis. Gene expression analysis by microarray was performed twice. FIG. 6B shows mRNA expression level of Md and Mt2 in different CD4⁺ T cell subsets at 72 h was analyzed by RT-PCR. The data are representative of two independent experiments.

FIGS. 7A-7C. FIG. 7A shows flow cytometry results of WT and Mt^{-/-} naïve CD4⁺ T cells which were differentiated with TGF-β1 and IL-6 for 72 h. The frequency of CD4⁺IL-17-IFN-γ, IL-10-producing cells was determined by flow cytometry. FIG. 7B shows naïve CD4⁺ T cells were differentiated towards Th17 or Tr1 cells with TGF-β1 (2 ng/ml) and IL-6 (20 ng/ml) or IL-27 (50 ng/ml) with or without purified MT1 or MT2 proteins (1 μM). The frequency of IL-17- and IL-10-expressing cells was determined by flow cytometry after 72 h of culture, showing that MT1 and MT2 decreased the differentiation of CD4+ T cells towards Tr1 CD4+ phenotype, either in the presence of IL-27 or TGFβ and IL-6; FIG. 7C shows the frequency of IL-10 and IFN-γ-secreting cells from WT or Mlt^{-/-} CD4⁺ T cell activated with anti-CD3/CD28 in the presence of the indicated cytokines (vitamin D3, Dex, and the combination of Vitamin D3+Dex). The data are representative of three independent experiments.

FIGS. 8A-8D. FIG. 8A shows mRNA levels assessed by RT-PCT of Mt1e, Mt1h and Il10 of naïve human CD4⁺CD45RO⁻CD62L⁺ T cells which were isolated from blood of healthy donors and were differentiated towards Th0 and Tr1 cells for 5 days, showing high levels of Mt1e and Mt1h in Tr1 cells. FIG. 8B shows the frequency of IL-10- and IFN-γ-expressing cells as determined by flow cytometry after 5 days of culture of naïve human CD4⁺CD45RO⁻CD62L⁺ T cells which were differentiated towards Tr1 cells with IL-27 with or without purified MT1 or MT2 proteins (1 μM). The data are representative of two independent experiments; FIG. 8C Naive human CD4⁺CD45RO⁻ CD62L⁺ T cells were differentiated towards Tr1 cells with IL-27 with or without recombinant MT1 or MT2 proteins (1 μM). The cultured supernatants were collected on day 5, and IL-10 and IFN-γ secretions were determined by ELISA. The data demonstrates that MT1 and MT2 decreases IL-10 production from differentiated human Tr1 cells. FIG. 8D shows naive human CD4⁺CD45RO⁻ CD62L⁺ T cells were incubated with the plate-bound anti-CD3/CD28 (2 μg/ml) in the presence or absence of IL-27 (100 μg/ml) with or without MTs (1 μM) for 3 days. Cell-free culture supernatants were transferred to freshly purified CD4⁺ T cells culture with plate-bound anti-CD3/CD28 (1 μg/ml). Neutralizing anti-IL-10 antibody (20 μg/ml) was added to the indicated condition and proliferation was measured at day 5 by thymidine incorporation. Data represent one of two experiments involving seven randomly selected donors, and the error bars represent the mean SD.

FIGS. 9A-9B shows CD4⁺ T cells which were isolated from lymph nodes of MOG₃₅₋₅₅ immunized WT and Mt^{-/-} mice, and subsequently re-stimulated in vitro with MOG₃₅₋₅₅ and IL-23 (effector cells) or IL-27 (Tr1 cells). FIG. 9A shows EAE development in WT (129/Sv B6 F1) recipients adoptively transferred with WT MOG₃₅₋₅₅ specific effector T cells with (5:1) or without WT or Mt^{-/-} Tr1 cells. FIG. 9B shows the data from FIG. 9A expressed linear-regression curve of the disease score over time.

FIGS. 10A-10B shows results from 2D2 transgenic Tr1 cells were activated with MOG and IL-23 in vitro (effector T cells cells), or stimulated with MOG and IL-27 and transduced with control (Ctrl RV) or MT1-expressing (MT1 RV) retrovirus (Tr1 cells). GFP⁺ cells were FACS-sorted after 3 d and co-transferred into animal models with effector cells. FIG. 10A shows EAE disease scores of WT (C57BL/6J) recipient mice at indicated times after transfer. FIG. 10B shows the data from FIG. 10A expressed as a linear-regression curve of the disease score over time.

FIG. 11 shows CD8⁺ Tim-3⁺PD-1⁺ TILs (CD8+ T cells) exhibit dysfunctional/exhausted phenotype. Cytokine production in tumor infiltrating lymphocytes from CT26 colon carcinoma-bearing mice. TILs were harvested from CT26 tumor-bearing mice and stimulated with PMA and Ionomycin prior to intracytoplasmic cytokine staining Expression of cytokine in Tim-3⁻PD-1⁻, Tim-3⁻PD-1⁺and Tim-3⁺PD-1⁺CD8⁺ TILs. Data shown are representative of five independent analyses.

FIGS. 12A-12B shows Metallothionein 1 and 2 are upregulated in CD8⁺ Tim-3⁺PD-1⁺tumor infiltrating lymphocytes (e.g., CD8+ T cells) that exhibit dysfunctional/exhausted phenotype. FIG. 12A shows the mean expression signal for Metallothionein 1 (Mt1) and 2 (Mt2) in CD8⁺tumor infiltrating lymphocytes (TILs) from CT26 colon carcinoma (n=2) and CD8⁺CD44hi memory T cells from a naïve tumor free Balb/c mouse (n=2) as determined by whole genome expression profiling. Mt1 and Mt2 are up-regulated 37 and 11 fold respectively. FIG. 12B shows the expression of Mt1 and Mt2 as determined by quantitative PCR in CD8⁺ TILs from CT26 and CD8⁺CD44hi memory T cells from a naïve tumor free Balb/c mouse. Samples (n=2) are independent from FIG. 12A.

FIGS. 13A-13B shows a T cell intrinsic effect of metallothionein 1/2 deficiency. FIG. 13A shows data relating to tumor growth in individual Rag−/− mice that were implanted with 5×10⁵ B16F10 melanoma cells, which were reconstituted with wild type (WT) or metallothionein deficient (KO) T cells. FIG. 13B shows splenocytes from mice melanoma tumor bearing WT and KO mice from (A) were stimulated with 10 uM hgp100. After 48 hrs, ^{3H}-Thymidine was added to cultures for the determination of proliferation. Each bar represents the mean of triplicate wells for an individual mouse from each group.

FIG. 14 shows defective Tim3, PD-1 expression in MT KO CD8⁺ T cells in response to IL-2. Naïve CD8⁺ T cells from either wild type (WT) or metallothionein deficient mice (KO) mice were cultured in the presence of 2 ug/ml anti-CD3 and irradiated APC. After 48 h, IL-2 (10 ng/ml) or IL-27 (20 ng/ml) were added to cultures. Cells were analyzed three days later by flow cytometry.

FIG. 15 shows a defect in development of dysfunctional/exhausted phenotype in MT KO CD8+ T cells in response to IL-27. Naïve CD8⁺ T cells from either wild type (WT) or metallothionein deficient mice (KO) mice were cultured in the presence of 2 ug/ml anti-CD3 and irradiated APC. After 48 h, IL-2 (10 ng/ml) or IL-27 (20 ng/ml) were added to cultures. Cytokine production was analyzed three days later by intracytoplasmic staining

FIG. 16 shows the induction of CD8+ T cell dysfunction/exhaustion gene signature in response to IL-27 is defective in Mt^{-/-} CD8⁺ T cells. Naïve CD8⁺ T cells from either wild type (WT) or metallothionein deficient mice (MT^{-/-}) mice were cultured in the presence of 2 ug/ml anti-CD3 and irradiated APC. After 48 h, IL-27 (20 ng/ml) was added to cultures. Cells were harvested three days later and gene expression analyzed using Nanostring encounter technology using a custom codeset.

FIGS. 17A-17C shows MT1/2 deficiency results in slower tumor progression and improved responses to tumor antigens. FIG. 17A shows mean tumor growth in metallothionein 1/2 deficient mice inoculated with 5×10⁵ B16F10 melanoma cells. FIGS. 17B, 17C show responses to stimulation with hgp100 in tumor-infiltrating lymphocytes and tumor-draining lymph node.

FIGS. 18A-18B show improved expression of Granzyme B in MT KO CD8 TILs after stimulation with tumor antigen. TILs were isolated from wildtype or metallothionein 1/2 deficient mice bearing B16F10 melanoma. TILs were stimulated with hgp100 for 5 hours prior to examination of Granzyme B expression by flow cytometry. FIG. 18A shows representative flow data showing expression of Granzyme B in CD8+ T cells. FIG. 18B shows summary data for Granzyme B expression in CD8+ T cells from wildtype (n=4) and Metallothionein 1/2 deficient (n=3) mice.

FIG. 19 shows IL-27 induces Metallothionein 1 and 2 in CD8+ T cells. Naïve CD8+ T cells were cultured with or without IL-27 (20 ng/ml). At 24 hours, CD8+ T cells were harvested and gene expression analyzed using Nanostring ncounter technology.

### DETAILED DESCRIPTION

The compositions and methods described herein are based, in part, on the novel discovery that metallothioneins (MTs) are negative regulators of IL-10-producing type 1 regulatory (Tr1) CD4+ cells, and that inhibition of MTs can increase IL-10 production from Tr1 cells, and/or promote or increase their differentiation from CD4+ cells and/or increase the proliferation or activity of Tr1 CD4+ cells. One aspect of the present invention relates to methods and compositions comprising inhibitors of MTs to increase IL-10 production from Tr1 CD4+ cells, for example, in the treatment of autoimmune diseases and treatment of graft-versus-hosts disease and to prevent transplant rejection in a transplant recipient subject. In another aspect of the present invention, overexpression or activation of MTs in Tr1 CD4+ cells can be used to decrease Tr1 CD4+ T cell proliferation and/or differentiation from CD4+ T cells, for example in diseases where a lot of IL-10 is produced, for example, during chronic immune conditions. In such embodiments, overexpression or activation of MTs in Tr1 CD4+ cells can be performed ex vivo, and the cells administered to a subject in need thereof, e.g., a subject where it is desirable to decrease IL-10 production from Tr1 CD4+ cells.

Furthermore, the inventors have also discovered that MTs regulate different populations of T-cells in different ways. For example, the inventors have discovered that inhibitors of MT1 and/or MT2 can increase exhausted or dysfunctional CD8+ T-cell proliferation and/or activity and can also decrease CD8+ T-cell exhaustion. Accordingly, another aspect of the present invention relates to methods and compositions comprising inhibitors of MTs to decrease CD8+ T cell exhaustion in a subject in need thereof, or to decrease exhausted CD8+ T cell and/or increase CD8+ T cell activity in a subject in need thereof, such as, for example, a subject with a chronic immune condition, e.g., a subject with a chronic infection or cancer.

Furthermore, the inventors have demonstrated that when Tr1 CD4+ cells treated ex vivo with MT activators, e.g., exogenous MT protein, or overexpression of MT protein, are transplanted into an animal model of EAE, the Tr1 cells no longer have the ability to produce IL-10, and are unable to suppress autoimmunity and/or protect the animal from the EAE disease. Thus, activators of MTs can decrease the proliferation or activity of Tr1 CD4+ cells and/or decrease their differentiation from CD4+ cells, thus reducing the production of IL-10 from Tr1 CD4+ cells.

Accordingly, provided herein are compositions comprising at least one MT modulator, such as a agonist and/or activator and inhibitor and/or antagonist, and methods thereof for modulating chronic immune conditions, such as cancer, infections, and autoimmune disorders, as described in more detail herein below.

### DEFINITIONS

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

As used herein, the terms “autoimmune disease” or “auto-immune disease” and “autoimmune-related disease” are used interchangeably, and refer to a disease or condition that occurs when the body tissues are attacked by its own immune system. The immune system is a complex organization within the body that is designed normally to “seek and destroy” invaders of the body, including infectious agents. Subjects with autoimmune diseases frequently have unusual antibodies circulating in their blood that target their own body tissues. Autoimmune diseases also include diseases with immunoregulatory abnormalities, such as for example a wide variety of autoimmune and chronic inflammatory diseases, including systemic lupus erythematosis, chronic rheumatoid arthritis, type 1 diabetes mellitus, inflammatory bowel disease, biliary cirrhosis, uveitis, multiple sclerosis, graft versus host (GVH) disease and other disorders such as Crohn's disease, ulcerative colitis, bullous pemphigoid, sarcoidosis, psoriasis, ichthyosis, and Graves ophthalmopathy. Although the underlying pathogenesis of each of these conditions can be quite different, they have in common the appearance of a variety of autoantibodies and/or self-reactive lymphocytes, and are therefore auto-immune disease which can be treated with the compositions and methods as disclosed herein. In particular, immune diseases involving dysregulation of CD4+ Tr1 cells or exhaustion of CD8+ T cells can be treated according to the methods described herein.

One commonly known auto-immune disease is graft versus host disease caused by graft versus host reaction (GVH reaction) in which immune cells within a transplant or graft attack the recipient's body. Subjects that are recipients of bone marrow transplants or organ transplants, either before transplantation, at the time of transplantation or post transplantation can be treated with the compositions and methods as disclosed herein. Thus, the methods and compositions as disclosed herein a MT1 and/or MT2 modulator as disclosed herein, e.g., can be administered to subjects in need of, or scheduled to receive immune suppression (i.e. in need of immunosuppressive therapy), for example subjects who are organ or bone marrow transplant recipients or subjects having, or at risk of developing an auto-immune disease.

As used herein, the term “immunosuppressive agents” is meant any composition capable of suppressing the immune system, and includes analogs, hydrolysis products, metabolites, and precursors of an immunosuppressive agent unless the context precludes it. In some embodiments, immunosuppressive agents useful in the compositions and methods as disclosed herein can be selected from one of the following compounds: mycophenolic acid, cyclosporin, azathioprine, tacrolimus, cyclosporin A, FK506, rapamycin, leflunomide, deoxyspergualin, prednisone, azathioprine, mycophenolate mofetil, OKT3, ATAG or mizoribine. One example of such a composition is cyclosporine.

The term “inflammatory disorders” or “inflammatory diseases” as used herein refer to disorders or conditions mediated by an inflammatory cytokine cascade, defined herein as an in vivo release from cells of at least one pro-inflammatory cytokine in a subject, wherein the cytokine release affects a physiological condition of the subject. Non limiting examples of cells that produce proinflammatory cytokines are monocytes, macrophages, neutrophils, epithelial cells, osteoblasts, fibroblasts, smooth muscle cells, and neurons.

As used herein, a “cytokine” is a generic term for proteins released by any of the lymph cells that act on other cells as intercellular mediators and affect cellular activity and control inflammation. Cytokines are typically soluble proteins or peptides which are naturally produced by mammalian cells and which act in vivo as humoral regulators at micro- to picomolar concentrations. Cytokines can, either under normal or pathological conditions, modulate the functional activities of individual cells and tissues. A proinflammatory cytokine is a cytokine that is capable of causing any of the following physiological reactions associated with inflammation: vasodilatation, hyperemia, increased permeability of vessels with associated edema, accumulation of granulocytes and mononuclear phagocytes, or deposition of fibrin. In some cases, the pro-inflammatory cytokine can also cause apoptosis, such as in chronic heart failure, where TNF has been shown to stimulate cardiomyocyte apoptosis. Nonlimiting examples of pro-inflammatory cytokines are tumor necrosis factor (TNF), interleukin (IL)-1.alpha., IL-1.beta., IL-6, IL-8, IL-18, interferon-γ (INFγ), HMG-1, platelet-activating factor (PAF), and macrophage migration inhibitory factor (MIF). In preferred embodiments of the invention, the pro-inflammatory cytokine that is inhibited by cholinergic agonist treatment is TNF, an IL-1, IL-6 or IL-18, because these cytokines are produced by macrophages and mediate deleterious conditions for many important disorders, for example endotoxic shock, asthma, rheumatoid arthritis, inflammatory bile disease, heart failure, and allograft rejection. In most preferred embodiments, the pro-inflammatory cytokine is TNF. Pro-inflammatory cytokines are to be distinguished from anti-inflammatory cytokines, such as IL-4, IL-10, and IL-13, which are not mediators of inflammation. In some embodiments, release of anti-inflammatory cytokines is not inhibited by cholinergic agonists. Additionally examples of cytokines include, lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance (MIS); mouse gonadotropin-associated peptide; inhibin; activin; vascular endothelial growth factor (VEGF); integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as, for example and not for limitation, IL-1, IL-1α, IL-1.beta., IL-2, IL-3, IL-4, IL-5, IL-6, IL-7, IL-8, IL-9, IL-10, IL-11, IL-12; a tumor necrosis factor such as TNF-α or TNF-β; and other polypeptide factors including leukemia inhibitory factor (LIF) and kit ligand (KL). As used herein, when referring to a patient the term “cytokine” refers to on one or more of those produced by the patient.

As used herein, an “immune response” refers to a response by a cell of the immune system, such as a B cell, T cell (CD4+ or CD8+), regulatory T cell, antigen-presenting cell, dendritic cell, monocyte, macrophage, NKT cell, NK cell, basophil, eosinophil, or neutrophil, to a stimulus. In some embodiments of the aspects described herein, the response is specific for a particular antigen (an “antigen-specific response”), and refers to a response by a CD4 T cell, CD8 T cell, or B cell via their antigen-specific receptor. In some embodiments of the aspects described herein, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response.

As used herein, the terms “functional exhaustion” or “unresponsiveness” refers to a state of a cell where the cell does not perform its usual function or activity in response to normal input signals, and includes refractivity of immune cells to stimulation, such as stimulation via an activating receptor or a cytokine. Such a function or activity includes, but is not limited to, proliferation or cell division, entrance into the cell cycle, cytokine production (e.g., IL-27 induced production of IL-10 by Tr1 cells), cytotoxicity, trafficking, phagocytotic activity, or any combination thereof. Normal input signals can include, but are not limited to, stimulation via a receptor (e.g., T cell receptor, B cell receptor, co-stimulatory receptor). Unresponsive immune cells can have a reduction of at least 10%, 20%, 30%, 40%, 50%, 60%, 70%, 80%, 90%, 95%, or even 100% in cytotoxic activity, cytokine production, proliferation, trafficking, phagocytotic activity, or any combination thereof, relative to a corresponding control immune cell of the same type. In some particular embodiments of the aspects described herein, a cell that is functionally exhausted is a CD8+ T cell that expresses the CD8+ cell surface marker. Such CD8+ cells normally proliferate and produce cell killing enzymes, e.g., they can release the cytotoxins perforin, granzymes, and granulysin.

As used herein, the term “unresponsiveness” also includes refractivity to activating receptor-mediated stimulation. Such refractivity is generally antigen-specific and persists after exposure to the antigen has ceased. Unresponsive immune cells can have a reduction of at least 10%, 20%, 30%, 40%, 50%, 60%, 70%, 80%, 90%, 95%, or even 100% in cytotoxic activity, cytokine production, proliferation, trafficking, phagocytotic activity, or any combination thereof, relative to a corresponding control immune cell of the same type.

As used herein, the term “reduces T cell tolerance” means that a given treatment or set of conditions leads to reduced T cell tolerance, i.e., greater T cell activity, responsiveness, and/or ability or receptiveness with regards to activation. In some embodiments, a reduced T cell tolerance is a reduced CD8+ T cell tolerance. Methods of measuring T cell activity are known in the art. By way of non-limiting example, T cell tolerance can be induced by contacting T cells with recall antigen, anti-CD3 in the absence of costimulation, and/or ionomycin. Levels of, e.g. LDH-A, RAB10, and/or ZAP70 (both intracellular or secreted) can be monitored, for example, to determine the extent of T cell tolerogenesis (with levels of IL-2, interferon-γ and TNF correlating with increased T cell tolerance). The response of cells pre-treated with, e.g. ionomycin, to an antigen can also be measured in order to determine the extent of T cell tolerance in a cell or population of cells, e.g. by monitoring the level of secreted and/or intracellular IL-2 and/or TNF-α (see, e.g. Macian et al. Cell 2002 109:719-731; which is incorporated by reference herein in its entirety). Other characteristics of T cells having undergone adaptive tolerance is that they have increased levels of Fyn and ZAP-70/Syk, Cbl-b, GRAIL, Ikaros, CREM (cAMP response element modulator), B lymphocyte-induced maturation protein-1 (Blimp-1), PD1, CD5, and SHP2; increased phosphorylation of ZAP-70/Syk, LAT, PLCγ1/2, ERK, PKC-Θ/IKBA; increased activation of intracellular calcium levels; decreased histone acetylation or hypoacetylation and/or increased CpG methylation at the IL-2 locus. Thus, in some embodiments, modulation of one or more of any of these parameters can be assayed to determine whether one or more MT1 or MT2 modulating agents modulates an immune response in vivo or modulates immune tolerance.

The term “agent” or “compound” as used herein refers to a chemical entity or biological product, or combination of chemical entities or biological products, administered to a subject to treat or prevent or control a disease or condition. The chemical entity or biological product is preferably, but not necessarily a low molecular weight compound, but may also be a larger compound, or any organic or inorganic molecule effective in the given situation, including modified and unmodified nucleic acids such as antisense nucleic acids, RNAi, such as siRNA or shRNA, peptides, peptidomimetics, receptors, ligands, and antibodies, aptamers, polypeptides, nucleic acid analogues or variants thereof. Examples include an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof. Agents can be selected from a group comprising: chemicals; small molecules; nucleic acid sequences; nucleic acid analogues; proteins; peptides; aptamers; antibodies; or fragments thereof. A nucleic acid sequence can be RNA or DNA, and can be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA), modified RNA (mod-RNA) etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides etc. A protein and/or peptide or fragment thereof can be any protein of interest, for example, but are not limited to: mutated proteins; therapeutic proteins and truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof. Alternatively, the agent can be intracellular within the cell as a result of introduction of a nucleic acid sequence into the cell and its transcription resulting in the production of the nucleic acid and/or protein modulator of a MTr gene within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

As used herein, “gene silencing” or “gene silenced” in reference to an activity of an RNAi molecule, for example a siRNA or miRNA refers to a decrease in the mRNA level in a cell for a target gene (e.g. a MT gene) by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene. By way of an example only, in some embodiments RNAi agents which serve to inhibit or gene silence are useful in the methods, kits and compositions disclosed herein to inhibit a MT (e.g., MT1 and/or MT2) gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 116:281-297), comprises a dsRNA molecule.

The term “nucleic acid” is well known in the art. A “nucleic acid” as used herein will generally refer to a molecule (i.e., strand) of DNA, RNA or a derivative or analog thereof, comprising a nucleobase. A nucleobase includes, for example, a naturally occurring purine or pyrimidine base found in DNA (e.g. an adenine “A,” a guanine “G.” a thymine “T” or a cytosine “C”) or RNA (e.g. an A, a G. an uracil “U” or a C). The term “nucleic acid” encompasses the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length. The term “nucleic acid” also refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. The terms “polynucleotide sequence” and “nucleotide sequence” are also used interchangeably herein.

As used herein, the term “gene” refers to a nucleic acid comprising an open reading frame encoding a polypeptide, including both exon and (optionally) intron sequences. A “gene” refers to coding sequence of a gene product, as well as non-coding regions of the gene product, including 5′UTR and 3′UTR regions, introns and the promoter of the gene product. The coding region of a gene can be a nucleotide sequence coding for an amino acid sequence or a functional RNA, such as tRNA, rRNA, catalytic RNA, siRNA, miRNA and antisense RNA. A gene can also be an mRNA or cDNA corresponding to the coding regions (e.g. exons and miRNA) optionally comprising 5′- or 3′ untranslated sequences linked thereto. These definitions generally refer to a single-stranded molecule, but in specific embodiments will also encompass an additional strand that is partially, substantially or fully complementary to the single-stranded molecule. Thus, a nucleic acid may encompass a double-stranded molecule or a double-stranded molecule that comprises one or more complementary strand(s) or “complement(s)” of a particular sequence comprising a molecule. As used herein, a single stranded nucleic acid may be denoted by the prefix “ss”, a double stranded nucleic acid by the prefix “ds”, and a triple stranded nucleic acid by the prefix “is.” The term “gene” refers to the segment of DNA involved in producing a polypeptide chain, it includes regions preceding and following the coding region as well as intervening sequences (introns and non-translated sequences, e.g., 5′- and 3′-untranslated sequences and regulatory sequences) between individual coding segments (exons). A gene can also be an amplified nucleic acid molecule produced in vitro comprising all or a part of the coding region and/or 5′- or 3′-untranslated sequences linked thereto.

A “promoter” is a region of a nucleic acid sequence at which initiation and rate of transcription are controlled. It may contain elements at which regulatory proteins and molecules may bind, such as RNA polymerase and other transcription factors, to initiate the specific transcription of a nucleic acid sequence.

The term “enhancer” refers to a cis-acting regulatory sequence involved in the transcriptional activation of a nucleic acid sequence. An enhancer can function in either orientation and may be upstream or downstream of the promoter.

As used herein, the term “gene product(s)” is used to refer to include RNA transcribed from a gene (e.g., mRNA), or a polypeptide encoded by a gene or translated from RNA.

The terms “polypeptide” and “protein” are used interchangeably to refer to a polymer of amino acid residues, and are not limited to a minimum length. Peptides, oligopeptides, dimers, multimers, and the like, are also composed of linearly arranged amino acids linked by peptide bonds, and whether produced biologically, recombinantly, or synthetically and whether composed of naturally occurring or non-naturally occurring amino acids, are included within this definition. Both full-length proteins and fragments thereof are encompassed by the definition. The terms also include co-translational and post-translational modifications of the polypeptide, such as, for example, disulfide-bond formation, glycosylation, acetylation, phosphorylation, proteolytic cleavage (e.g., cleavage by furins or metalloproteases and prohormone convertases (PCs)), and the like. Furthermore, for purposes of the present invention, a “polypeptide” encompasses a protein that includes modifications, such as deletions, additions, and substitutions (generally conservative in nature as would be known to a person in the art), to the native sequence, as long as the protein maintains the desired activity. These modifications can be deliberate, as through site-directed mutagenesis, or can be accidental, such as through mutations of hosts that produce the proteins, or errors due to PCR amplification or other recombinant DNA methods. Polypeptides or proteins are composed of linearly arranged amino acids linked by peptide bonds, but in contrast to peptides, has a well-defined conformation. Proteins, as opposed to peptides, generally consist of chains of 50 or more amino acids. For the purposes of the present invention, the term “peptide” as used herein typically refers to a sequence of amino acids of made up of a single chain of D- or L-amino acids or a mixture of D- and L-amino acids joined by peptide bonds. Generally, peptides contain at least two amino acid residues and are less than about 50 amino acids in length.

The incorporation of non-natural amino acids, including synthetic non-native amino acids, substituted amino acids, or one or more D-amino acids into the peptides (or other components of the composition, with exception for protease recognition sequences) is desirable in certain situations. D-amino acid-containing peptides exhibit increased stability in vitro or in vivo compared to L-amino acid-containing forms. Thus, the construction of peptides incorporating D-amino acids can be particularly useful when greater in vivo or intracellular stability is desired or required. More specifically, D-peptides are resistant to endogenous peptidases and proteases, thereby providing better oral trans-epithelial and transdermal delivery of linked drugs and conjugates, improved bioavailability of membrane-permanent complexes (see below for further discussion), and prolonged intravascular and interstitial lifetimes when such properties are desirable. The use of D-isomer peptides can also enhance transdermal and oral trans-epithelial delivery of linked drugs and other cargo molecules. Additionally, D-peptides cannot be processed efficiently for major histocompatibility complex class II-restricted presentation to T helper cells, and are therefore less likely to induce humoral immune responses in the whole organism. Peptide conjugates can therefore be constructed using, for example, D-isomer forms of cell penetrating peptide sequences, L-isomer forms of cleavage sites, and D-isomer forms of therapeutic peptides. In some embodiments, a MT1 and/or MT2 modulator comprises a MT1 or MT2 protein or fragment thereof fused to a Fc fragment, which is comprised of D- or L-amino acid residues, as use of naturally occurring L-amino acid residues has the advantage that any break-down products should be relatively non-toxic to the cell or organism.

In yet a further embodiment, a MT1 and/or MT2 modulator which is a peptide or fragments or derivatives thereof can be a retro-inverso peptide. A “retro-inverso peptide” refers to a peptide with a reversal of the direction of the peptide bond on at least one position, i.e., a reversal of the amino- and carboxy-termini with respect to the side chain of the amino acid. Thus, a retro-inverso analogue has reversed termini and reversed direction of peptide bonds while approximately maintaining the topology of the side chains as in the native peptide sequence. The retro-inverso peptide can contain L-amino acids or D-amino acids, or a mixture of L-amino acids and D-amino acids, up to all of the amino acids being the D-isomer. Partial retro-inverso peptide analogues are polypeptides in which only part of the sequence is reversed and replaced with enantiomeric amino acid residues. Since the retro-inverted portion of such an analogue has reversed amino and carboxyl termini, the amino acid residues flanking the retro-inverted portion are replaced by side-chain-analogous a-substituted geminal-diaminomethanes and malonates, respectively. Retro-inverso forms of cell penetrating peptides have been found to work as efficiently in translocating across a membrane as the natural forms. Synthesis of retro-inverso peptide analogues are described in Bonelli, F. et al., Int J Pept Protein Res. 24(6):553-6 (1984); Verdini, A and Viscomi, G. C., J. Chem. Soc. Perkin Trans. 1:697-701 (1985); and U.S. Pat. No. 6,261,569, which are incorporated herein in their entirety by reference. Processes for the solid-phase synthesis of partial retro-inverso peptide analogues have been described (EP 97994-B) which is also incorporated herein in its entirety by reference.

The term “antibody” is meant to be an immunoglobulin protein that is capable of binding an antigen. Antibody as used herein is meant to include antibody fragments, e.g. F(ab′)₂, Fab′, Fab, capable of binding the antigen or antigenic fragment of interest.

The term “humanized antibody” is used herein to describe complete antibody molecules, i.e. composed of two complete light chains and two complete heavy chains, as well as antibodies consisting only of antibody fragments, e.g. Fab, Fab′, F(ab′)₂, and Fv, wherein the CDRs are derived from a non-human source and the remaining portion of the Ig molecule or fragment thereof is derived from a human antibody, preferably produced from a nucleic acid sequence encoding a human antibody.

The terms “human antibody” and “humanized antibody” are used herein to describe an antibody of which all portions of the antibody molecule are derived from a nucleic acid sequence encoding a human antibody. Such human antibodies are most desirable for use in antibody therapies, as such antibodies would elicit little or no immune response in the human subject.

The terms “subject” and “individual” are used interchangeably herein, and refer to an animal, for example a human, to whom treatment, including prophylactic treatment, with a pharmaceutical composition according to the present invention, is provided. The term “subject” as used herein refers to human and non-human animals. The term “non-human animals” and “non-human mammals” are used interchangeably herein, and includes all vertebrates, e g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. The term does not denote a particular age or sex. Thus, adult and newborn subjects, as well as fetuses, whether male or female, are intended to be covered. Examples of subjects include humans, dogs, cats, cows, goats, and mice. The term subject is further intended to include transgenic species.

The term “tissue” is intended to include intact cells, blood, blood preparations such as plasma and serum, bones, joints, muscles, smooth muscles, and organs.

The term “disease” or “disorder” is used interchangeably herein, refers to any alternation in state of the body or of some of the organs, interrupting or disturbing the performance of the functions and/or causing symptoms such as discomfort, dysfunction, distress, or even death to the person afflicted or those in contact with a person. A disease or disorder can also related to a distemper, ailing, ailment, malady, disorder, sickness, illness, complaint, indisposition, or affliction.

The term “chemokine” is a generic term for any of the proteins that act on white blood cells and induce them to move and/or become activated to carry out their immune system functions. Chemokines are well-known in the art. Exemplary chemokines include, for example and not for limitation, TECK, ELC, BLC-1, CTACK, RANTES, fractalkine, exotaxin, eotaxin-2, Monocyte chemoattractant protein-1 (MCP-1), MCP-2, MCP-3, MCP-4, MDC, leukotactin, SDF-1.beta., lymphotactin, TARC, ITAC, ENA-70, ENA-78, IP-10, NAP-2, interleukin-8 (IL-8), HCC-1, MIP-la, MIP-1β, MIP-1δ, 1-309, GRO-α, GRO-β, GRO-γ, MPIF-1, I-LINK, and GCP-2. As used herein, when referring to a patient the term “chemokine” refers to any of those produced by the patient.

A “pharmaceutical composition” refers to a composition that usually contains an excipient, such as a pharmaceutically acceptable carrier that is conventional in the art and that is suitable for administration to cells or to a subject. In addition, compositions for topical (e.g., oral mucosa, respiratory mucosa) and/or oral administration can be in the form of solutions, suspensions, tablets, pills, capsules, sustained-release formulations, oral rinses, or powders, as known in the art and described herein. The compositions also can include stabilizers and preservatives. For examples of carriers, stabilizers and adjuvants, University of the Sciences in Philadelphia (2005) Remington: The Science and Practice of Pharmacy with Facts and Comparisons, 21st Ed.

As used herein, the terms “treat,” “treating,” and “treatment” refer to the alleviation or measurable lessening of one or more symptoms or measurable markers of a disease or disorder; while not intending to be limited to such, disease or disorders of particular interest include autoimmune diseases, chronic infection and cancer. Measurable lessening includes any statistically significant decline in a measurable marker or symptom. In some embodiments, treatment is prophylactic treatment.

The term “therapeutically effective amount” refers to an amount effective, at dosages and for periods of time necessary, to achieve the desired therapeutic result, e.g., a diminishment or prevention of effects associated with various disease states or conditions, such as reduce a symptom of an autoimmune disease in the subject. The term “therapeutically effective amount” refers to an amount of a MT1 and/or MT2 modulator as disclosed herein effective to treat or prevent a disease or disorder in a mammal, preferably a human. In the case of treatment of an autoimmune and/or inflammatory disease, a therapeutically effective amount may alleviate one or more symptoms associated with the disease including decreasing or stabilizing pain, swelling, discomfort and/or tissue damage. As a non-limiting example, for rheumatoid arthritis, efficacy and response can represent achieving the American College of Rheumatology ACR20 or ACR50 scores. As another example, in Crohn's disease, therapeutically effective doses can lower the Disease Activity Index (CDAI). A therapeutically effective amount of a MT1 and/or MT2 modulator can vary according to factors such as the disease state, age, sex, and weight of the subject, and the ability of the therapeutic compound to elicit a desired response in the subject. A therapeutically effective amount is also one in which any toxic or detrimental effects of the therapeutic agent are outweighed by the therapeutically beneficial effects. In some embodiments, a therapeutically effective amount is an “effective amount”, which as used herein refers to the amount of therapeutic agent of pharmaceutical composition to alleviate at least one or some of the symptoms of the disease or disorder. An “effective amount” for purposes herein is thus determined by such considerations as are known in the art and is the amount to achieve improvement including, but not limited to, improved survival rate or more rapid recovery, or improvement or elimination of at least one symptom and other indicator of an immune or autoimmune disease which are appropriate measures by those skilled in the art. It should be noted that a MT1 and/or MT2 modulator as disclosed herein can be administered as a pharmaceutically acceptable salt and can be administered alone or as an active ingredient in combination with pharmaceutically acceptable carriers, diluents, adjuvants and vehicles.

The term “prophylactically effective amount” refers to an amount of a MT1 and/or MT2 modulator which is effective, at dosages and for periods of time necessary, to achieve the desired prophylactic result, e.g., the amount of a MT inhibitor to promote the secretion of IL-10 from Tr1 cells to prevent the onset of an autoimmune disease where the subject is at risk of developing an autoimmune disease, or for example, the amount of a MT inhibitor to decrease CD8+ T cell exhaustion to reduce a symptom of a chronic immune disease, e.g., a chronic infection or to treat cancer in the subject. Typically, since a prophylactic dose of a MT1 and/or MT2 modulator is administered to a subject prior to or at an earlier stage of an autoimmune disease, and in some embodiments, a prophylactically effective amount is less than the therapeutically effective amount. A prophylactically effective amount of a MT1 and/or MT2 modulator is also one in which any toxic or detrimental effects of the compound are outweighed by the beneficial effects.

As used herein, the terms “prevent,” “preventing” and “prevention” refer to the avoidance or delay in manifestation of one or more symptoms or measurable markers of a disease or disorder, e.g., of an autoimmune disease. A delay in the manifestation of a symptom or marker is a delay relative to the time at which such symptom or marker manifests in a control or untreated subject with a similar likelihood or susceptibility of developing the disease or disorder. The terms “prevent,” “preventing” and “prevention” include not only the avoidance or prevention of a symptom or marker of the disease, but also a reduced severity or degree of any one of the symptoms or markers of the disease, relative to those symptoms or markers in a control or non-treated individual with a similar likelihood or susceptibility of developing the disease or disorder, or relative to symptoms or markers likely to arise based on historical or statistical measures of populations affected by the disease or disorder. By “reduced severity” is meant at least a 10% reduction in the severity or degree of a symptom or measurable disease marker, relative to a control or reference, e.g., at least 15%, 20%, 30%, 40%, 50%, 60%, 70%, 80%, 90%, 95%, 99% or even 100% (i.e., no symptoms or measurable markers).

As used herein, the terms “administering,” and “introducing” are used interchangeably herein and refer to the placement of the agents of metabolic regulators of the present invention into a subject by a method or route which results in at least partial localization of a MT1 and/or MT2 modulator at a desired site. The compounds of the present invention can be administered by any appropriate route which results in an effective treatment in the subject. In some embodiments, administering is not systemic administration. In some embodiments, administration includes contacting a specific population of T cells ex vivo with a MT1 and/or MT2 modulator as disclosed herein, and administering the treated specific T cell population to a subject. For example, in some embodiments, a CD4+ Tr1 cell population is contacted with a MT1 and/or MT2 inhibitor, and the MT1/2 inhibitor treated Tr1 cells are administered to the subject, e.g., a subject in need of treatment, such as, for example, a subject with an autoimmune disease or a transplant recipient. In another example, in some embodiments, a CD8 T-cell population is contacted with a MT1 and/or MT2 inhibitor, and the MT1/2 inhibitor treated CD8+ T-cells are administered to a subject, e.g., a subject in need of treatment, such as, for example, a subject with a chronic immune diseases, e.g., a chronic infection and/or cancer.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration,” “administered systemically”, “peripheral administration” and “administered peripherally” as used herein mean the administration of a MT1 and/or MT2 modulator such that it enters the animal's system and, thus, is subject to metabolism and other like processes, for example, subcutaneous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, with which a MT modulator as described herein is combined in a formulation to be administered to a subject. Each carrier must be “acceptable” in the sense of being compatible with the other ingredients of the formulation, as well as in the sense of not being toxic or provoking undue side effects in an individual. Pharmaceutically acceptable carriers are well known to those of skill in the art.

The term “vectors” refers to a nucleic acid molecule capable of transporting another nucleic acid to which it has been linked; a plasmid is a species of the genus encompassed by “vector”. The term “vector” typically refers to a nucleic acid sequence containing an origin of replication and other entities necessary for replication and/or maintenance in a host cell. Vectors capable of directing the expression of genes and/or nucleic acid sequence to which they are operatively linked are referred to herein as “expression vectors”. In general, expression vectors of utility are often in the form of “plasmids” which refer to circular double stranded DNA loops which, in their vector form are not bound to the chromosome, and typically comprise entities for stable or transient expression or the encoded DNA. Other expression vectors can be used in the methods as disclosed herein for example, but are not limited to, plasmids, episomes, bacterial artificial chromosomes, yeast artificial chromosomes, bacteriophages or viral vectors, and such vectors can integrate into the host's genome or replicate autonomously in the particular cell. A vector can be a DNA or RNA vector. Other forms of expression vectors known by those skilled in the art which serve the equivalent functions can also be used, for example self replicating extrachromosomal vectors or vectors which integrates into a host genome. Preferred vectors are those capable of autonomous replication and/or expression of nucleic acids to which they are linked. Vectors capable of directing the expression of genes to which they are operatively linked are referred to herein as “expression vectors”.

The term “viral vectors” refers to the use as viruses, or virus-associated vectors as carriers of the nucleic acid construct into the cell. Constructs may be integrated and packaged into non-replicating, defective viral genomes like Adenovirus, Adeno-associated virus (AAV), or Herpes simplex virus (HSV) or others, including reteroviral and lentiviral vectors, for infection or transduction into cells. The vector may or may not be incorporated into the cells genome. The constructs may include viral sequences for transfection, if desired. Alternatively, the construct may be incorporated into vectors capable of episomal replication, e.g. EPV and EBV vectors.

As used herein, a “promoter” or “promoter region” or “promoter element” used interchangeably herein, refers to a segment of a nucleic acid sequence, typically but not limited to DNA or RNA or analogues thereof, that controls the transcription of the nucleic acid sequence to which it is operatively linked. The promoter region includes specific sequences that are sufficient for RNA polymerase recognition, binding and transcription initiation. This portion of the promoter region is referred to as the promoter. In addition, the promoter region includes sequences which modulate this recognition, binding and transcription initiation activity of RNA polymerase. These sequences may be cis-acting or may be responsive to trans-acting factors. Promoters, depending upon the nature of the regulation may be constitutive or regulated.

The term “regulatory sequences” is used interchangeably with “regulatory elements” herein refers element to a segment of nucleic acid, typically but not limited to DNA or RNA or analogues thereof, that modulates the transcription of the nucleic acid sequence to which it is operatively linked, and thus act as transcriptional modulators. Regulatory sequences modulate the expression of gene and/or nucleic acid sequence to which they are operatively linked. Regulatory sequence often comprise “regulatory elements” which are nucleic acid sequences that are transcription binding domains and are recognized by the nucleic acid-binding domains of transcriptional proteins and/or transcription factors, repressors or enhancers etc. Typical regulatory sequences include, but are not limited to, transcriptional promoters, inducible promoters and transcriptional elements, an optional operate sequence to control transcription, a sequence encoding suitable mRNA ribosomal binding sites, and sequences to control the termination of transcription and/or translation. Regulatory sequences can be a single regulatory sequence or multiple regulatory sequences, or modified regulatory sequences or fragments thereof. Modified regulatory sequences are regulatory sequences where the nucleic acid sequence has been changed or modified by some means, for example, but not limited to, mutation, methylation etc.

The term “operatively linked” as used herein refers to the functional relationship of the nucleic acid sequences with regulatory sequences of nucleotides, such as promoters, enhancers, transcriptional and translational stop sites, and other signal sequences. For example, operative linkage of nucleic acid sequences, typically DNA, to a regulatory sequence or promoter region refers to the physical and functional relationship between the DNA and the regulatory sequence or promoter such that the transcription of such DNA is initiated from the regulatory sequence or promoter, by an RNA polymerase that specifically recognizes, binds and transcribes the DNA. In order to optimize expression and/or in vitro transcription, it may be necessary to modify the regulatory sequence for the expression of the nucleic acid or DNA in the cell type for which it is expressed. The desirability of, or need of, such modification may be empirically determined Enhancers need not be located in close proximity to the coding sequences whose transcription they enhance. Furthermore, a gene transcribed from a promoter regulated in trans by a factor transcribed by a second promoter may be said to be operatively linked to the second promoter. In such a case, transcription of the first gene is said to be operatively linked to the first promoter and is also said to be operatively linked to the second promoter.

The term “reduced” or “reduce” or “decrease” as used herein generally means a decrease by a statistically significant amount relative to a reference. However, for avoidance of doubt, “reduced” means statistically significant decrease of at least 10% as compared to a reference level, for example a decrease by at least 20%, at least 30%, at least 40%, at least t 50%, or least 60%, or least 70%, or least 80%, at least 90% or more, up to and including a 100% decrease (i.e. absent level as compared to a reference sample), or any decrease between 10-100% as compared to a reference level, as that term is defined herein.

The term “low” as used herein generally means lower by a statically significant amount; for the avoidance of doubt, “low” means a statistically significant value at least 10% lower than a reference level, for example a value at least 20% lower than a reference level, at least 30% lower than a reference level, at least 40% lower than a reference level, at least 50% lower than a reference level, at least 60% lower than a reference level, at least 70% lower than a reference level, at least 80% lower than a reference level, at least 90% lower than a reference level, up to and including 100% lower than a reference level (i.e. absent level as compared to a reference sample).

The terms “increased” or “increase” as used herein generally mean an increase by a statically significant amount; for the avoidance of doubt, “increased” means a statistically significant increase of at least 10% as compared to a reference level, including an increase of at least 20%, at least 30%, at least 40%, at least 50%, at least 60%, at least 70%, at least 80%, at least 90%, at least 100% or more, including, for example at least 2-fold, at least 3-fold, at least 4-fold, at least 5-fold, at least 10-fold increase or greater as compared to a reference level, as that term is defined herein.

The term “high” as used herein generally means a higher by a statically significant amount relative to a reference; for the avoidance of doubt, “high” means a statistically significant value at least 10% higher than a reference level, for example at least 20% higher, at least 30% higher, at least 40% higher, at least 50% higher, at least 60% higher, at least 70% higher, at least 80% higher, at least 90% higher, at least 100% higher, at least 2-fold higher, at least 3-fold higher, at least 4-fold higher, at least 5-fold higher, at least 10-fold higher or more, as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

The term “optional” or “optionally” means that the subsequent described event, circumstance or substituent may or may not occur, and that the description includes instances where the event or circumstance occurs and instances where it does not.

The articles “a” and “an” are used herein to refer to one or to more than one (i.e., at least one) of the grammatical object of the article. By way of example, “an element” means one element or more than one element. Thus, in this specification and the appended claims, the singular forms “a,” “an,” and “the” include plural references unless the context clearly dictates otherwise. Thus, for example, reference to a pharmaceutical composition comprising “an agent” includes reference to two or more agents.

As used herein, the term “comprising” means that other elements can also be present in addition to the defined elements presented. The use of “comprising” indicates inclusion rather than limitation. The term “consisting of” refers to compositions, methods, and respective components thereof as described herein, which are exclusive of any element not recited in that description of the embodiment. As used herein the term “consisting essentially of” refers to those elements required for a given embodiment. The term permits the presence of elements that do not materially affect the basic and novel or functional characteristic(s) of that embodiment of the invention.

Other than in the operating examples, or where otherwise indicated, all numbers expressing quantities of ingredients or reaction conditions used herein should be understood as modified in all instances by the term “about.” The term “about” when used in connection with percentages can mean ±1%.

This invention is further illustrated by the examples which should not be construed as limiting. The contents of all references cited throughout this application, as well as the figures and tables are incorporated herein by reference. All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

### T Cell Populations

Tr1 Cells

Type 1 T regulatory (Tr1) cells are a subtype of CD4+ cells and are naturally occurring blood cells specialized to induce tolerance against environmental foreign antigens taken up by inhalation, ingestion or contact and certain tissue specific self-antigens. Tr1 cells have been shown to prevent autoimmune and chronic inflammatory diseases such as colitis, arthritis, and multiple sclerosis in different experimental animal models.

Tr1 cells home preferentially to the sites of injury and control inflammation by: (1) secreting natural immunosuppressive molecules such as Interleukin-10 (IL-10) and Interleukin-13 (IL-13), (2) initiating cell-cell contact mediated suppression through surface molecules such as CTLA-4 and GITR and (3) killing of myeloid cells via a Perforin/Granzyme B pathway. These multiple mechanisms of action lead to inhibition of pro-inflammatory cell activation, proliferation and cytokine production.

In their normal role, Tr1 cells suppress the immune response to environmental antigens (e.g., ingested, contact, inhaled antigens etc.) and several specific antigens. Tr1 cells have a therapeutic potential in the suppression of chronic inflammation (e.g., inflammatory Bowel Disease (IBD), rheumatoid arthritis (RA), multiple sclerosis (MS) and in the reduction of autoimmunity and prevention of transplant rejection.

CD8+ T Cells

A CD8+ T cell is a T cell expressing the CD8 cell surface marker, and recognizes antigens in the context of MHC class I presentation. CD8+ T cells have cytotoxic activity and proliferate in response to IFNγ and other cytokines. Engagement of CD8+ T-cell to the TCR receptor of a CD8+ T-cell antigen presented by Class I MHC molecules and costimulating molecules lead to cytotoxic activity, proliferation and/or cytokine production. Exhausted T cells do not respond to such TCR stimulation, as discussed herein in the definition section regarding “functional exhaustion”.

T cell exhaustion is a state of T cell dysfunction that arises during many chronic infections and cancer. It is defined by poor effector function, sustained expression of inhibitory receptors and a transcriptional state distinct from that of functional effector or memory T cells. Exhaustion prevents optimal control of infection and tumors.

### Metallothioneins (MTs)

Metallothioneins (MTs) are a class of ubiquitously occurring low molecular weight cysteine- and metal-rich proteins containing sulfur-based metal clusters. The conservation of these clusters in an increasing number of three-dimensional structures of invertebrate, vertebrate and bacterial MTs signifies the importance of this structural motif. It is becoming increasingly clear that mammalian MTs have diverse functions including involvement in zinc homeostasis, protection against heavy metal toxicity and oxidative damage. Mammalian MTs are single chain polypeptides of 61, 60 or 68 amino acid residues with an N-terminal acetylmethionine and often alanine at the carboxyl terminus. They contain 20 cysteine residues, which are central to the binding of metals. MTs have characteristic C-X-C, C-Y-C, and C-C sequences, where X and Y are non-cysteine amino acids. There are 7 bivalent ions for every 20 cysteines forming metal thiolate complexes in a two domain structure.

There are four MT subgroups, namely MT1, MT2, MT3, and MT4. The MT1 and MT2 isoforms, which differ by only a single negative charge, are the most widely expressed isoforms in different tissues. Human MT genes are clustered at a single locus on chromosome 16, and at least 14 of the 17 genes so far identified, are functional. These encode multiple isoforms of MT1 (MT1A, B, E, F, G, H, I, K, L and X), MT2, MT3 and MT4.

Stimuli that can induce MT expression include metals, hormones (e.g. glucocorticoids), cytokines, a variety of other chemicals, inflammation, and stress. MT degradation takes place mainly in the lysosomes. MT appears less susceptible to proteolysis in the metal bound state. In vivo, metal-MTs have far longer half-lives than apo-MT.

MT1 and MT2 are present throughout the liver, brain and spinal cord, and that the main cell type expressing these MT isoforms is the astrocyte; nevertheless, MT1 and MT2 expression was also found in ependymal cells, epithelial cells of choroid plexus, meningeal cells of the pia mater, and endothelial cells of blood vessels.

MTs are stress-inducible proteins that maintain metal homeostasis and scavenge free radicals. It is generally accepted that the major functions of MTs are related to metal metabolism. Postulated functions include detoxification and storage of heavy metals and the regulation of cellular copper and zinc metabolism in response to dietary and physiological changes. MT1 and MT2 deficient mice showed both increased oxidative stress and neuronal apoptosis during epileptic seizures, experimental autoimmune encephalomyelitis (EAE), and following traumatic brain injury. Likewise, transgenic MT1 overexpressing mice showed significantly reduced oxidative tissue damage and cell death during traumatic brain injury, focal cerebral ischemia, and 6-aminonicotinamide (6-AN)-induced brain stem toxicity. Furthermore, MT1 and MT2 improve the clinical outcome and reduce mortality in different CNS disorders (Penkowa et al., Biomed Rev, 2002, 13; 1-18). MT has recently been shown to mediate neuroprotection in genetically engineered mouse model of Parkinson's disease (Ebadi et al., 2005, 134; 67-75).

Metallothionein has been documented to bind a wide range of metals including cadmium, zinc, mercury, copper, arsenic, silver, etc. Metallation of MT was previously reported to occur cooperatively but recent reports have provided strong evidence that metal-binding occurs via a sequential, noncooperative mechanism. [6] The observation of partially metallated MT (that is, having some free metal binding capacity) suggest that these species are biologically important.

Metallothioneins likely participate in the uptake, transport, and regulation of zinc in biological systems. Mammalian MT binds three Zn(II) ions in its beta domain and four in the alpha domain. Cysteine is a sulfur-containing amino acid. However, the participation of inorganic sulfide and chloride ions has been proposed for some MT forms. In some MTs, mostly bacterial, histidine participates in zinc binding By binding and releasing zinc, metallothioneins (MTs) may regulate zinc levels within the body. Zinc, in turn, is a key element for the activation and binding of certain transcription factors through its participation in the zinc finger region of the protein. Metallothionein also carries zinc ions (signals) from one part of the cell to another. When zinc enters a cell, it can be picked up by thionein (which thus becomes “metallothionein”) and carried to another part of the cell where it is released to another organelle or protein. In this way the thionein-metallothionein becomes a key component of the zinc signaling system in cells. This system is particularly important in the brain, where zinc signaling is prominent both between and within nerve cells. It also seems to be important for the regulation of the tumor suppressor protein p53.

Where MTs play an important role in transcription factor regulation, problems with MT function or expression may lead to malignant transformation of cells and ultimately cancer. Studies have reported increased expression of MTs in some cancers of the breast, colon, kidney, liver, skin (melanoma), lung, nasopharynx, ovary, prostate, mouth, salivary gland, testes, thyroid and urinary bladder; they have also found lower levels of MT expression in hepatocellular carcinoma and liver adenocarcinoma. There are also reports that higher levels of MT expression may also lead to resistance to chemotherapeutic drugs. Heavy metal toxicity has been proposed as a hypothetical etiology of autism, and dysfunction of MT synthesis and activity may play a role in this. Many heavy metals, including mercury, lead, and arsenic have been linked to symptoms that resemble the neurological symptoms of autism.

Agonists (e.g., activators) and inhibitors (e.g. antagonists) of MT1 can be an agonist (e.g., activator) and inhibitor (e.g. antagonist) of a metallothionein (MT) selected from the group consisting of metallothionein-1A (MT1A), metallothionein-1B (MT1B), metallothionein-1E (MT1E), metallothionein-1F (MT1F), metallothionein-1G (MT1G), metallothionein-1H (MT1H), metallothionein-1I (MT1I), metallothionein-1 K (MT1K), metallothionein-1L (MT1L), metallothionein-1R (MT1R), metallothionein-1X (MT1X), metallothionein-2 (MT2), metallothionein-3 (MT3) and metallothionein-4 (MT4). The sequences of the latter mentioned metallothioneins are identified in the Gene Bank under the following Acc. Nos: Q9BQN2, P04731, P07438, P04732, P04733, P13640, P80294, P80295, P80296, Q93083, P80297, P02795, P25713, P47944, respectively. In some embodiments, an agonist (e.g., activator) and inhibitor (e.g. antagonist) of a metallothionein (MT) is an agonist (e.g., activator) and inhibitor (e.g. antagonist) of MT1h and MT1e.

Accordingly, the term “MT1A” as used herein refers to the 61amino acid protein having the amino acid sequence of:

(SEQ ID NO: 1)
MDPNCSCATG GSCTCTGSCK CKECKCTSCK KSCCSCCPMS
CAKCAQGCIC KGASEKCSCC,

as described by, e.g., NP_005937.2, together with any naturally occurring allelic, splice variants, and processed forms thereof. MTA1 is encoded by the following nucleic acid sequence:

| (SEQ ID NO: 2) | |
|---|---|
| 1 | accaagcctt ccacgtgcgc cttatagcct ctcaacttct tgcttgggat ctccaacctc |
| 61 | accgcggctc gaaatggacc ccaactgctc ctgcgccact ggtggctcct gcacctgcac |
| 121 | tggctcctgc aaatgcaaag agtgcaaatg cacctcctgc aagaagagct gctgctcctg |
| 181 | ctgccccatg agctgtgcca agtgtgccca gggctgcatc tgcaaagggg catcagagaa |
| 241 | gtgcagctgc tgtgcctgat gtccggacag ccctgctcga agatatagaa agagtgacct |
| 301 | gcacaaactt ggaatttttt ttccatacaa ccctgaccca tttactgtat tttttttaat |
| 361 | gaaatatgtg aatgataata aaagttgctg acttaaaaaa aaaaaaaaaa aaaaaaaaaa |
| 421 | aaaaaaaaaa aaaaaaaaaa aaaaaaaaaa aaaaaaaaaa aaaaaaaa |

as described by, e.g., NM_005946.2 GI:71274112 (MT1A), together with any naturally occurring allelic, splice variants, and processed forms thereof.

According to the invention an inhibitor or activator of a MT for use in the methods and compositions as disclosed herein is selected from the group consisting of metallothionein-1 A (MT1A), metallothionein-1B (MT1B), metallothionein-1E (MT1E), metallothionein-1F (MT1F), metallothionein-1G (MT1G), metallothionein-1H (MT1H), metallothionein-1I (MT1I), metallothionein-1 K (MT1K), metallothionein-1L (MT1L), metallothionein-1R (MT1R), metallothionein-1X (MT1X), metallothionein-2 (MT2), metallothionein-3 (MT3) and metallothionein-4 (MT4). The sequences of the latter mentioned metallothioneins are identified in the Gene Bank under the following Acc. Nos: Q9BQN2, P04731, P07438, P04732, P04733, P13640, P80294, P80295, P80296, Q93083, P80297, P02795, P25713, P47944, respectively.

In some embodiments, an inhibitor or activator of MT1 is an inhibitor or activator of a MT1 isoform as shown in Table 1.

TABLE 1
Isoforms of human MT1

| | | Amino acid | Nucleic acid |
|---|---|---|---|
| MT1 | | sequence | sequence |
| isoform | MT1 isoform name | Accession No. | Accession No. |
| MT1A | metallothionein 1A | NP_005937.2 | NM_005946.2 |
| | | (SEQ ID NO: 1) | (SEQ ID NO: 2) |
| MT1B | metallothionein 1B | NP_005938.1 | NM_005947.2 |
| | | (SEQ ID NO: 3) | (SEQ ID NO: 4) |
| MT1E | metallothionein 1E | NP_783316.2 | NM_175617.3 |
| | | (SEQ ID NO: 5) | (SEQ ID NO: 6) |
| MT1F | metallothionein 1F | NP_005940.1 | NM_005949.3 |
| | | (SEQ ID NO: 7) | (SEQ ID NO: 8) |
| MT1G | metallothionein 1G | NP_005941.1 | NM_005950.1 |
| | | (SEQ ID NO: 9) | (SEQ ID NO: 10) |
| MT1H | metallothionein 1H | NP_005942.1 | NM_005951.2 |
| | | (SEQ ID NO: 11) | (SEQ ID NO: 12) |
| MT1IP | metallothionein 1I, | n/a | NR_003669.1 |
| | pseudogene | | (SEQ ID NO: 13) |
| MT1JP | metallothionein 1J, | n/a | NM_175622.3 |
| | pseudogene | | (SEQ ID NO: 14) |
| MT1L | metallothionein 1L | n/a | NR_001447.2 |
| | (gene/pseudogene) | | (SEQ ID NO: 15) |
| MT1M | metallothionein 1M | NP_789846.1 | NM_176870.2 |
| | | (SEQ ID NO: 16) | (SEQ ID NO: 17) |
| MT1X | metallothionein 1X | NP_005943.1 | NM_005952.3 |
| | | (SEQ ID NO: 18) | (SEQ ID NO: 19) |
| MT1CP | metallothionein 1C, | n/a | NG_005880.3 |
| | pseudogene | | (SEQ ID NO: 20) |
| MT1DP | metallothionein 1D, | A1L3X4 | NR_027781.1 |
| | pseudogene | (SEQ ID NO: 21) | (SEQ ID NO: 22) |
| MT1HL1 | metallothionein 1H- | n/a | NM_001039954.2 |
| | like 1 | | (SEQ ID NO: 23) |
| MT1P1 | metallothionein 1 | n/a | NG_005527.3 |
| | pseudogene 1 | | (SEQ ID NO: 24) |
| MT1P3 | metallothionein 1 | n/a | NG_005652.3 |
| | pseudogene 3 | | (SEQ ID NO: 25) |
| MT1XP1 | metallothionein 1X | n/a | Gene ID: 645652 |
| (MTL1) | pseudogene 1 | | (SEQ ID NO: 26) |

Accordingly, the term “MT2” as used herein refers to the 61amino acid protein having the amino acid sequence of:

(SEQ ID NO: 27)
MDPNCSCAAG DSCTCAGSCK CKECKCTSCK KSCCSCCPVG
CAKCAQGCIC KGASDKCSCC,

together with any naturally occurring allelic, splice variants, and processed forms thereof. MTA2 is encoded by the following nucleic acid sequence:

| (SEQ ID NO: 28) | |
|---|---|
| 1 | cttgccgcgc tgcactccac cacgcctcct ccaagtccca gcgaacccgc gtgcaacctg |
| 61 | tcccgactct agccgcctct tcagctcgcc atggatccca actgctcctg cgccgccggt |
| 121 | gactcctgca cctgcgccgg ctcctgcaaa tgcaaagagt gcaaatgcac ctcctgcaag |
| 181 | aaaagctgct gctcctgctg ccctgtgggc tgtgccaagt gtgcccaggg ctgcatctgc |
| 241 | aaaggggcgt cggacaagtg cagctgctgc gcctgatgct gggacagccc cgctcccaga |
| 301 | tgtaaagaac gcgacttcca caaacctgga ttttttatgt acaaccctga ccgtgaccgt |
| 361 | ttgctatatt cctttttcta tgaaataatg tgaatgataa taaaacagct ttgacttgaa |
| 421 | aaaaaaaaaa aaaaaaaaaa aaaaaaaaaa aaaaaaaaaa aaaaaa |

as described by, e.g., NM_005953.3 or GI:205277384 (MT2A, MT-II), together with any naturally occurring allelic, splice variants, and processed forms thereof.
Modulation of IL-10 Production from Tr1 Cells

As used herein, “modulating” or “to modulate” generally means either reducing or inhibiting the activity of MT1 and/or MT2, or alternatively increasing the activity of, a target or antigen, such as MT1 and/or MT2, as measured using a suitable in vitro, cellular or in vivo assay, such as those described herein in the Examples. In particular, “modulating” or “to modulate” can mean either reducing or inhibiting the activity of, or alternatively increasing a (relevant or intended) biological activity of, a target or antigen, e.g., MT1 and/or MT2, as measured using a suitable in vitro, cellular or in vivo assay (which will usually depend on the target or antigen involved), by at least 5%, at least 10%, at least 25%, at least 50%, at least 60%, at least 70%, at least 80%, or 90% or more, compared to activity of the target or antigen in the same assay under the same conditions but without the presence of the inhibitor/antagonist agents or activator/agonist agents described herein.

As will be clear to the skilled person, “modulating” can also involve effecting a change (which can either be an increase or a decrease) in affinity, avidity, specificity and/or selectivity of a target or antigen (e.g., MT1 and/or MT2) for one or more of its ligands (e.g., zinc or other metals), binding partners, partners for association into a homomultimeric or heteromultimeric form, or substrates; and/or effecting a change (which can either be an increase or a decrease) in the sensitivity of the target or antigen (e.g., MT1 and/or MT2) for one or more conditions in the medium or surroundings in which the target or antigen is present (such as pH, ion strength, the presence of co-factors, etc.), compared to the same conditions but without the presence of a modulating agent. Again, this can be determined in any suitable manner and/or using any suitable assay known per se, depending on the target or antigen involved. In particular, an action as an inhibitor/antagonist or activator/agonist can be such that an intended biological or physiological activity is increased or decreased, respectively, by at least 5%, at least 10%, at least 25%, at least 50%, at least 60%, at least 70%, at least 80%, or 90% or more, compared to the biological or physiological activity in the same assay under the same conditions but without the presence of the inhibitor/antagonist agent or activator/agonist agent. Modulating can, for example, also involve allosteric modulation of the target or antigen (e.g., MTs); and/or reducing or inhibiting the binding of the target or antigen (e.g., MTs) to one of its substrates or ligands (e.g., zinc or other metals) and/or competing with a natural ligand, substrate for binding to the target or antigen. Modulating can also involve activating the target or antigen or the mechanism or pathway in which it is involved. Modulating can for example also involve effecting a change in respect of the folding or conformation of the target or antigen, or in respect of the ability of the target or antigen to fold, to change its conformation (for example, upon binding of a ligand), to associate with other (sub)units, or to disassociate. Such a change will have a functional effect.

### Inhibitors or Antagonists of MTs

As discussed herein, the inventors have discovered that inhibition of MTs, e.g., MT1 and/or MT2 is useful to increase IL-10 production from Tr1 cells, e.g., for use in methods for the treatment of subjects with autoimmune diseases, or transplant recipients. In alternative embodiments, inhibitors of MTs (e.g., MT1 and/or MT2 inhibitors) are useful for promoting the differentiation of CD4+ cells to Tr1 CD4+ cells, and/or for increasing the proliferation of Tr1 CD4+ cells in a subject. In another embodiment, inhibitors of MT (e.g., MT1 and/or MT2 inhibitors) are useful in promoting the activity of Tr1 cells, e.g., where such activities include, but are not limited to: secretion of IL-10 and IL-13, homing of Tr1 cells to sites of inflammation, killing of myeloid cells via Granzyme B release, and inhibition of pro-inflammatory cell activation and proliferation and cytokine function.

In another embodiment, inhibitors of MTs (e.g., MT1 and/or MT2 inhibitors) are useful for decreasing CD8+ T-cell exhaustion, e.g., for treatment of a subject with a chronic immune disease, e.g., a chronic infection and/or cancer. In some embodiments, an inhibitor of MTs (e.g., MT1 and/or MT2 inhibitors) can be used to increase the activity of functionally exhausted CD8+ T-cells (such that the CD8+ cells proliferate in response to cytokines such as IFNγ, have cytotoxic activity, etc.) and/or increase the differentiation of functionally exhausted CD8+ cells.

In some embodiments, an inhibitor of a MT, e.g., MT1 and/or MT2 is a protein inhibitor, and in some embodiments, the inhibitor is any agent which inhibits the function of MT, e.g., MT1 and/or MT2 or the expression of the MT, e.g., MT1 and/or MT2 from its gene.

RNAi Inhibitors of MTs.

Inhibition of MTs, e.g., MT1 and/or MT2 gene can be by gene silencing RNAi molecules according to methods commonly known by a skilled artisan. For example, a gene silencing siRNA oligonucleotide duplexes targeted specifically to human MTs can readily be used to knockdown MT gene expression. MT1 and/or MT2 mRNA can be successfully targeted using siRNAs; and other siRNA molecules may be readily prepared by those of skill in the art based on the known sequence of the target mRNA. To avoid doubt, the sequence of a human MT1 isoforms and MT2 isoforms are provided herein.

An inhibitor of MT1 and/or MT2 can be any agent which inhibits the function of MT1 and/or MT2, such as antibodies, gene silencing RNAi molecules and the like. Commercial neutralizing antibodies of MT1 and/or MT2 are encompassed for use in the methods and compositions as disclosed herein. Additionally, small molecules agonists of MT1 and/or MT2 which are known by one of ordinary skill in the art and are encompassed for use in the methods and compositions as disclosed herein as an inhibitor of MT1 and/or MT2 protein function or their expression from the MT1 and/or MT2 genes.

In some embodiments a protein, or protein fragment or polypeptide of MT1 and/or MT2 can be used as an inhibitor of MT1 and/or MT2 in the methods, compositions and kits as disclosed herein. In some embodiments, a protein or protein fragment may be a protein, peptide or protein fragment of at least 10 amino acid sequence of MT1 and/or MT2 protein. In some embodiments, an inhibitor of MT1 and/or MT2 is a fragment or polypeptide of MT1 and/or MT2 which functions as a dominant negative or decoy molecule for ligands (e.g., zinc or other metals) binding to endogenous MT1 and/or MT2, and therefore a fragment of the MT1 and/or MT2 polypeptide can inhibit the function of endogenous MT1 and/or MT2 expressed in cells.

Accordingly, fragment of the MT1 and/or MT2 protein can be used to function as a dominant negative protein inhibitor of MT1 and/or MT2, respectively.

In some embodiments, a dominant negative inhibitor of MT1 and/or MT2 is at least about carboxyl most 50 amino acids of SEQ ID NO: 1-28, or at least about 40, or at least about 30 or at least about 20 or at least about 10 or more most-carboxyl amino acids of the MT1 and/or MT2 proteins sequences as disclosed herein. In some embodiments, a dominant negative inhibitor of MT1 and/or MT2 is at least about the N-terminal most 50 amino acids, or at least about 40, or at least about 30 or at least about 20 or at least about 10 or more most-carboxyl amino acids of the MT1 and/or MT2 proteins sequences as disclosed herein.

Accordingly, in some aspects, provided herein, are compositions comprising MT inhibitors or antagonists (e.g. MT1 and/or MT2 MT inhibitors or antagonists) for increasing IL-27-induction of IL-10 from Tr1 cells (e.g., for the treatment of autoimmune diseases and graft-versus-host-disease), and/or for use in decreasing CD8+ T cell exhaustion (e.g., for the treatment of chronic immune diseases and cancer).

As used herein, the terms “MT1 and/or MT2 inhibitor,” “MT1 and/or MT2 antagonist,” “MT1 and/or MT2 inhibitor agent,” and “MT1 and/or MT2antagonist agent” refer to a molecule or agent that significantly blocks, inhibits, reduces, or interferes with MT1 and/or MT2 (mammalian, such as human MT1 and/or MT2) biological activity in vitro, in situ, and/or in vivo, including activity of downstream pathways mediated by MT1 and/or MT2 signaling, such as, for example, transcription factor induction (e g., inhibition of the phosphorylation of STAT1 and STAT3), IL-10 induction, and/or elicitation of a cellular response to IL-27 or MT1 and/or MT2. Exemplary MT1 and/or MT2 inhibitors contemplated for use in the various aspects and embodiments described herein include, but are not limited to, anti-MT1 and/or anti-MT2 antibodies or antigen-binding fragments thereof that specifically bind to MT1 and/or MT2; anti-sense molecules directed to a nucleic acid encoding either MT1 and/or MT2; short interfering RNA (“siRNA”) molecules directed to a nucleic acid encoding one or both MT1 and/or MT2; or MT1 and/or MT2 inhibitory compound; RNA or DNA aptamers that bind to MT1 and/or MT2 and inhibit/reduce/block MT1 and/or MT2 mediated signaling; MT1 and/or MT2 structural analogs; soluble MT1 and/or MT2 proteins or fusion polypeptides thereof; anti-MT1 and/or anti-MT2 antibodies or antigen-binding fragments thereof; and small molecule agents that target or bind to MT1 and/or MT2. In some embodiments of these aspects and all such aspects described herein, a MT1 and/or MT2 inhibitor (e.g., an antibody or antigen-binding fragment thereof) binds (physically interacts with) MT1 and/or MT2, binds to an MT1 and/or MT2, targets downstream MT1 and/or MT2 signaling, and/or inhibits (reduces) MT1 and/or MT2 synthesis, production or release. In some embodiments of these aspects and all such aspects described herein, an MT1 and/or MT2 inhibitor binds MT1 and/or MT2 and prevents its binding to its ligands (e.g., zinc or other metals). In some embodiments of these aspects and all such aspects described herein, a MT1 and/or MT2 inhibitor specifically reduces or eliminates expression (i.e., transcription or translation) of MT1 and/or MT2.

In some embodiments of the compositions and methods described herein, the MT1 and/or MT2 inhibitor inhibits MT1 and/or MT2 mediated signal transduction. In some embodiments of the compositions and methods described herein, the MT1 and/or MT2 inhibitor targets MT1 and/or MT2 mediated transcription factor induction or activation, for example, STAT1 and/or STAT3 deactivation.

In some embodiments of the compositions and methods described herein, a MT inhibitor (e.g., MT1 and/or MT2 inhibitor) increases IL-27-mediated IL-10 production from Tr1 cells. In some embodiments of the compositions and methods described herein, a MT inhibitor (e.g., MT1 and/or MT2 inhibitor) increases STAT1 and/or STAT3 activation or phosphorylation. In some embodiments of the compositions and methods described herein, a MT inhibitor (e.g., MT1 and/or MT2 inhibitor) targets and increases IL-27-induced IL-10 production.

In some embodiments of the compositions and methods described herein, a MT inhibitor (e.g., MT1 and/or MT2 inhibitor) is an antibody or antigen-binding fragment thereof that selectively binds or physically interacts with MT1 and/or MT2. In some embodiments of the compositions and methods described herein, a MT inhibitor (e.g., MT1 and/or MT2 inhibitor) is an antibody or antigen-binding fragment thereof that binds to MT1 and/or MT2 and inhibits and/or blocks and/or prevents the function of MT1 and/or MT2.

In some embodiments of the compositions and methods described herein, the binding sites of a MT inhibitor (e.g., MT1 and/or MT2 inhibitor), such as an antibody or antigen-binding fragment thereof, are directed against MT metal (e.g., zinc) interaction site. In some embodiments of the compositions and methods described herein, the binding sites of a MT inhibitor (e.g., MT1 and/or MT2 inhibitor) are directed against a site on a target in the proximity of the ligand interaction site, in order to provide steric hindrance for the interaction of the target (e.g., zinc) with the MT enzyme. By binding to a MT-metal interaction site, a MT inhibitor (e.g., MT1 and/or MT2 inhibitor) described herein can reduce or inhibit the activity or expression of MT1 and/or MT2, and increase IL-27 signaling consequences, e.g., IL-10 induction, and/or elicitation of a cellular response to IL-27). In some embodiments of the compositions and methods described herein, a MT inhibitor (e.g., MT1 and/or MT2 inhibitor) is an anti-sense molecule directed to a nucleic acid encoding either MT1 and/or MT2. In some embodiments of the compositions and methods described herein, a MT inhibitor (e.g., MT1 and/or MT2 inhibitor) is a short interfering RNA molecule directed to a nucleic acid encoding acid encoding MT1 and/or MT2. In some embodiments of the compositions and methods described herein, a MT inhibitor (e.g., MT1 and/or MT2 inhibitor) is an RNA or DNA aptamer that binds to MT1 and/or MT2. In some embodiments of the compositions and methods described herein, a MT inhibitor (e.g., MT1 and/or MT2 inhibitor) is a small molecule compound or agent that targets or binds to MT1 and/or MT2.

As used herein, a MT inhibitor (e.g., MT1 and/or MT2 inhibitor) or antagonist has the ability to increase the activity and/or expression of IL-10 in a cell (e.g., T cells, such as CD8+ or CD4+ T cells or Tr1 cells) by at least 5%, at least 10%, at least 20%, at least 30%, at least 40%, at least 50%, at least 60%, at least 70%, at least 80%, at least 90%, at least 95%, at least 98%, at least 99%, or more, relative to the activity or expression level in the absence of a MT inhibitor (e.g., MT1 and/or MT2 inhibitor) or antagonist.

In some embodiments of the compositions and methods described herein, a MT inhibitor (e.g., MT1 and/or MT2 inhibitor) is an antibody or antigen-binding fragment thereof that binds MT1 and/or MT2. In other words, in some embodiments of the compositions and methods described herein, a MT inhibitor (e.g., MT1 and/or MT2 inhibitor) is an antibody or antigen-binding fragment thereof that binds to an epitope found in the MT1 and/or MT2 proteins. In some embodiments of the compositions and methods described herein, a MT inhibitor (e.g., MT1 and/or MT2 inhibitor) is an antibody or antigen-binding fragment thereof that binds or physically interacts with heterodimeric MT1 and/or MT2.

Exemplary assays to measure inhibition or reduction of downstream MT1 and/or MT2 activation, e.g., an increase in phosphorylating of STAT1 and/or STAT3 are known to those of ordinary skill in the art and are provided herein in the Examples.

In some embodiments of the compositions and methods described herein, a MT inhibitor (e.g., MT1 and/or MT2 inhibitor) or antagonist is a monoclonal antibody.

In some embodiments of the compositions and methods described herein, a MT inhibitor (e.g., MT1 and/or MT2 inhibitor) or antagonist is an antibody fragment or antigen-binding fragment. The terms “antibody fragment,” “antigen binding fragment,” and “antibody derivative” as used herein, refer to a protein fragment that comprises only a portion of an intact antibody, generally including an antigen binding site of the intact antibody and thus retaining the ability to bind antigen, and as described elsewhere herein.

In some embodiments of the compositions and methods described herein, a MT inhibitor (e.g., MT1 and/or MT2 inhibitor) or antagonist is a chimeric antibody derivative of MT1 and/or MT2 antagonist antibody or antigen-binding fragment thereof.

A MT inhibitor (e.g., MT1 and/or MT2 inhibitor) inhibitor or antagonist antibodies and antigen-binding fragments thereof described herein can also be, in some embodiments, a humanized antibody derivative.

In some embodiments, a MT inhibitor (e.g., MT1 and/or MT2 inhibitor) or antagonist antibodies and antigen-binding fragments thereof described herein, include derivatives that are modified, i.e., by the covalent attachment of any type of molecule to the antibody, provided that the covalent attachment does not prevent the antibody from binding to the target antigen.

In some embodiments of the compositions and methods described herein, completely human antibodies are used, which are particularly desirable for the therapeutic treatment of human patients.

In some embodiments of the compositions and methods described herein, a MT inhibitor (e.g., MT1 and/or MT2 inhibitor) or antagonist is a small molecule inhibitor or antagonist, including, but is not limited to, small peptides or peptide-like molecules, soluble peptides, and synthetic non-peptidyl organic or inorganic compounds. A small molecule inhibitor or antagonist can have a molecular weight of any of about 100 to about 20,000 daltons (Da), about 500 to about 15,000 Da, about 1000 to about 10,000 Da. In some embodiments of the compositions and methods described herein, a MT inhibitor (e.g., MT1 and/or MT2 inhibitor) or antagonist comprises a small molecule that binds MT1 and/or MT2. Exemplary sites of small molecule binding include, but are not limited to, the portion of MT1 and/or MT2 that binds to the its ligand, e.g., metal e.g., zinc or which are responsible in whole or in part for establishing and/or maintaining the correct three-dimensional conformation of the MT1 and/or MT2 enzyme. In some embodiments of the compositions and methods described herein, a MT inhibitor (e.g., MT1 and/or MT2 inhibitor) or antagonist comprises a small molecule that inhibits a MT1 and/or MT2 biological activity.

In some embodiments of the compositions and methods described herein, a MT inhibitor (e.g., MT1 and/or MT2 inhibitor) or antagonist is an RNA or DNA aptamer that binds or physically interacts with MT1 and/or mT2, and blocks interactions between MT1 and/or MT2 and its ligand, e.g., metal e.g., zinc.

In some embodiments of the compositions and methods described herein, a MT inhibitor (e.g., MT1 and/or MT2 inhibitor) or antagonist comprises at least one MT1 and/or MT2 structural analog. The terms MT1 and/or MT2 structural analogs, as used herein, refer to compounds that have a similar three dimensional structure as part of that of MT1 and/or MT2 and which bind to MT1 and/or MT2 under physiological conditions in vitro or in vivo, wherein the binding at least partially inhibits MT1 and/or MT2 biological activity. Suitable MT1 and/or MT2 structural analogs can be designed and synthesized through molecular modeling of MT1 and/or MT2. The MT1 and/or MT2 structural analogs can be monomers, dimers, or higher order multimers in any desired combination of the same or different structures to obtain improved affinities and biological effects.

In some embodiments of the compositions and methods described herein, a MT inhibitor (e.g., MT1 and/or MT2 inhibitor) or antagonist comprises at least one soluble MT1 and/or MT2 enzyme or fusion polypeptide thereof. In some such embodiments, the soluble MT1 and/or MT2 is fused to an immunoglobulin constant domain, such as an Fc domain.

In some embodiments of the compositions and methods described herein, a MT inhibitor (e.g., MT1 and/or MT2 inhibitor) inhibitor or antagonist comprises at least one antisense molecule capable of blocking or decreasing the expression of functional MT1 and/or MT2 by targeting nucleic acids encoding MT1 and/or MT2 as disclosed herein. Methods are known to those of ordinary skill in the art for the preparation of antisense oligonucleotide molecules that will specifically bind one or more of MTs (e.g., any isoform of MT1 and/or MT2) without cross-reacting with other polynucleotides. Exemplary sites of targeting include, but are not limited to, the initiation codon, the 5′ regulatory regions, including promoters or enhancers, the coding sequence, including any conserved consensus regions, and the 3′ untranslated region. In some embodiment of these aspects and all such aspects described herein, the antisense oligonucleotides are about 10 to about 100 nucleotides in length, about 15 to about 50 nucleotides in length, about 18 to about 25 nucleotides in length, or more. In certain embodiments, the oligonucleotides further comprise chemical modifications to increase nuclease resistance and the like, such as, for example, phosphorothioate linkages and 2′-O-sugar modifications known to those of ordinary skill in the art.

In some embodiments of the compositions and methods described herein, a MT inhibitor (e.g., MT1 and/or MT2 inhibitor) or antagonist comprises at least one siRNA molecule capable of blocking or decreasing the expression of functional isoforms of MT1 and/or MT2 by targeting nucleic acids encoding MT1 and/or MT2 isoforms. It is routine to prepare siRNA molecules that will specifically target one or more of MT1 and/or MT2 isoforms mRNA without cross-reacting with other polynucleotides. siRNA molecules for use in the compositions and methods described herein can be generated by methods known in the art, such as by typical solid phase oligonucleotide synthesis, and often will incorporate chemical modifications to increase half life and/or efficacy of the siRNA agent, and/or to allow for a more robust delivery formulation. Alternatively, siRNA molecules are delivered using a vector encoding an expression cassette for intracellular transcription of siRNA.

In some embodiments, a MT inhibitor (e.g., MT1 and/or MT2 inhibitor) or antagonists for use in the compositions and methods described herein is a PKC inhibitor, as disclosed in Yu et al., “Metal-induced metallothionein gene expression can be inactivated by a protein kinase C inhibitor” FEBS Letts, 1997; 420; 69-73, which is incorporated herein in its entirety by reference. PKC inhibitors are well known in the art, and include, for example, H7 and chelerthrine. A PKC inhibitor for use in the methods and compositions as disclose herein can be, for example, Bisindolylmaleimide I (LY333531), PMA (phorbol 12-myristate 13-acetate) (TPA; 12-O-tetradecanoyl-phorbol-13-acetate), staurosporine, PKC412 (midstaurin), UCN01, Go6976, byrostatin 1, Tamoxifen, LY317615 (enzasautaurin), Ro31-8220, Ro-32-043, GF109203X, ISIS3521 (aprinocarsen) and ISIS9606.

A PKC inhibitor can be assessed using a method and screen to assess the antagonist activity of a PKC inhibitor activity of a compound according to the methods as disclosed in U.S. Pat. No. 5,776,685, which is incorporated herein in its entirety by reference. The term “PKC inhibitor” or “PKC antagonist” as used herein refers to an agent that reduces or attenuates the biological activity of the PKC polypeptide in a cell, either by decreasing the activity of the PKC polypeptide or by effectively reducing the amount of PKC polypeptide in a cell or by decreasing the enzymatic activity of the PKC polypeptide. A “PKC inhibitor” thus refers to a molecule having the ability to inhibit a biological function of a native PKC, as well as a mutant PKC protein. Compounds that are PKC inhibitors include all solvates, hydrates, pharmaceutically acceptable salts, tautomers, stereoisomers, and prodrugs of the compounds. While in some embodiments PKC inhibitors herein specifically interact with, e.g. bind to, a PKC, molecules that inhibit PKC biological activity by interacting with other members of the PKC signal transduction pathway are also specifically included within this definition. Useful PKC inhibitors may selectively inhibit PKC, or may selectively inhibit calcium-independent or novel PKC isoforms. In some embodiments, PKC biological activity is inhibited by a PKC inhibitor as disclosed herein. Some PKC inhibitors may function by more than one mechanism to inhibit overall PKC activity in a cell.

In alternative embodiments, a MT inhibitor (e.g., MT1 and/or MT2 inhibitor) or antagonists for use in the compositions and methods described herein, can be an agent which inhibits MT synthesis as disclosed in Satoh et al., “Modulation of Resistance to anticancer drugs by inhibition of Metallothionein synthesis” Cancer Res., 1994; 54; 5255-5257), which is incorporated herein in its entirety by reference. In some embodiments, a MT inhibitor which inhibits MT synthesis is propargylglycine (PPG), a specific inhibitor of cystathionase (Abeles et al., J Am. Chem. Soc., 95; 6124-6125, 1973).

A MT inhibitor (e.g., MT1 and/or MT2 inhibitor) or antagonists for use in the compositions and methods described herein can be identified or characterized using methods known in the art, such as protein-protein binding assays, biochemical screening assays, immunoassays, and cell-based assays, which are well known in the art, including, but not limited to, those described herein in the Examples.

For example, to identify a molecule that inhibits interaction between MT1 and/or MT2 and its ligand, e.g., metals, e.g., zinc can be used. For example, MT1 and/or MT2 IL-27 polypeptide is immobilized on a microtiter plate by covalent or non-covalent attachment. The assay is performed by adding the non-immobilized component (ligand or receptor polypeptide), which can be labeled by a detectable label, to the immobilized component, in the presence or absence of the testing molecule. When the reaction is complete, the non-reacted components are removed and binding complexes are detected. If formation of binding complexes is inhibited by the presence of the testing molecule, the testing molecule can be deemed a candidate antagonist that inhibits binding between MT1 and/or MT2 and its receptor. Cell-based or membrane-based assays can also be used to identify a MT inhibitor (e.g., MT1 and/or MT2 inhibitor) or antagonists. For example, MT1 and/or MT2 can be added to a cell along with the testing molecule to be screened for a particular activity (e.g., binding to a metal, e.g., zinc), and the ability of the testing molecule to inhibit the activity of interest indicates that the testing molecule is a MT inhibitor (e.g., MT1 and/or MT2 inhibitor) or antagonist. In other embodiments, by detecting and/or measuring levels of MT1 and/or MT2 gene expression, antagonist molecules that inhibit MT1 and/or MT2 isoform gene expression can be tested. MT1 and/or MT2 isoform gene expression can be detected and/or measured by a variety of methods, such as real time RT-PCR, enzyme-linked immunosorbent assay (“ELISA”), Northern blotting, or flow cytometry, and as known to one of ordinary skill in the art.

Agents in General which Function as Inhibitors of MT1 and/or MT2

### Nucleic Acid Inhibitors of IGPR-1 Regulator Gene.

In some embodiments, inhibition of MT1 and/or MT2 is by an agent. One can use any agent, for example but are not limited to nucleic acids, nucleic acid analogues, peptides, phage, phagemids, polypeptides, peptidomimetics, ribosomes, aptamers, antibodies, small or large organic or inorganic molecules, or any combination thereof.

Agents useful in the methods as disclosed herein can also inhibit gene expression (i.e. suppress and/or repress the expression of the gene). Such agents are referred to in the art as “gene silencers” and are commonly known to those of ordinary skill in the art. Examples include, but are not limited to a nucleic acid sequence, for an RNA, DNA or nucleic acid analogue, and can be single or double stranded, and can be selected from a group comprising nucleic acid encoding a protein of interest, oligonucleotides, nucleic acids, nucleic acid analogues, for example but are not limited to peptide nucleic acid (PNA), pseudo-complementary PNA (pc-PNA), locked nucleic acids (LNA) and derivatives thereof etc. Nucleic acid agents also include, for example, but are not limited to nucleic acid sequences encoding proteins that act as transcriptional repressors, antisense molecules, ribozymes, small inhibitory nucleic acid sequences, for example but are not limited to RNAi, shRNAi, siRNA, micro RNAi (miRNA), antisense oligonucleotides, etc.

In some embodiments, an inhibitor of MT1 and/or MT2 is a RNAi agent. One of ordinary skill can select a RNAi agent to be used which inhibits the expression of MT1 and/or MT2 as disclosed herein.

As used herein, agents useful in the method as inhibitors of MT1 and/or MT2 gene expression and/or inhibition of MT1 and/or MT2 protein function can be any type of entity, for example but are not limited to chemicals, nucleic acid sequences, nucleic acid analogues, proteins, peptides or fragments thereof. In some embodiments, the agent is any chemical, entity or moiety, including without limitation, synthetic and naturally-occurring non-proteinaceous entities. In certain embodiments the agent is a small molecule having a chemical moiety.

In alternative embodiments, agents useful in the methods as disclosed herein are proteins and/or peptides or fragment thereof, which inhibit the gene expression of MT1 and/or MT2 or the function of the MT1 and/or MT2 protein. Such agents include, for example but are not limited to protein variants, mutated proteins, therapeutic proteins, truncated proteins and protein fragments. Protein agents can also be selected from a group comprising mutated proteins, genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof. As disclosed herein, a protein which inhibit the function of MT1 and/or MT2 is a soluble extracellular dominant negative MT1 and/or MT2 protein, or a functional fragment or variant thereof which inhibits wild-type full length MT1 and/or MT2 function.

Alternatively, agents useful in the methods as disclosed herein as inhibitors of MT1 and/or MT2 can be a chemicals, small molecule, large molecule or entity or moiety, including without limitation synthetic and naturally-occurring non-proteinaceous entities. In certain embodiments the agent is a small molecule having the chemical moieties as disclosed herein.

In some embodiments, agents that inhibit MT1 and/or MT2 is a nucleic acid. Nucleic acid inhibitors of MT1 and/or MT2 include, for example, but not are limited to, RNA interference-inducing (RNAi) molecules, for example but are not limited to siRNA, dsRNA, stRNA, shRNA and modified versions thereof, where the RNA interference (RNAi) molecule silences the gene expression from the IGPR-1 gene.

Accordingly, in some embodiments, inhibitors of MT1 and/or MT2 can inhibit MT1 and/or MT2 by any “gene silencing” methods commonly known by persons of ordinary skill in the art. In some embodiments, the nucleic acid inhibitor of MT1 and/or MT2 is an anti-sense oligonucleic acid, or a nucleic acid analogue, for example but are not limited to DNA, RNA, peptide-nucleic acid (PNA), pseudo-complementary PNA (pc-PNA), or locked nucleic acid (LNA) and the like. In alternative embodiments, the nucleic acid is DNA or RNA, and nucleic acid analogues, for example PNA, pcPNA and LNA. A nucleic acid can be single or double stranded, and can be selected from a group comprising nucleic acid encoding a protein of interest, oligonucleotides, PNA, etc. Such nucleic acid sequences include, for example, but are not limited to, nucleic acid sequence encoding proteins that act as transcriptional repressors, antisense molecules, ribozymes, small inhibitory nucleic acid sequences, for example but are not limited to RNAi, shRNAi, siRNA, micro RNAi (mRNAi), antisense oligonucleotides etc.

In some embodiments single-stranded RNA (ssRNA), a form of RNA endogenously found in eukaryotic cells can be used to form an RNAi molecule. Cellular ssRNA molecules include messenger RNAs (and the progenitor pre-messenger RNAs), small nuclear RNAs, small nucleolar RNAs, transfer RNAs and ribosomal RNAs. Double-stranded RNA (dsRNA) induces a size-dependent immune response such that dsRNA larger than 30 bp activates the interferon response, while shorter dsRNAs feed into the cell's endogenous RNA interference machinery downstream of the Dicer enzyme.

RNA interference (RNAi) provides a powerful approach for inhibiting the expression of selected target polypeptides. RNAi uses small interfering RNA (siRNA) duplexes that target the messenger RNA encoding the target polypeptide for selective degradation. siRNA-dependent post-transcriptional silencing of gene expression involves cutting the target messenger RNA molecule at a site guided by the siRNA.

RNA interference (RNAi) is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex (termed “RNA induced silencing complex,” or “RISC”) that recognizes and cleaves target mRNAs. RNAi can also be initiated by introducing nucleic acid molecules, e.g., synthetic siRNAs or RNA interfering agents, to inhibit or silence the expression of target genes. As used herein, “inhibition of target gene expression” includes any decrease in expression or protein activity or level of the target gene or protein encoded by the target gene as compared to a situation wherein no RNA interference has been induced. The decrease can be at least 30%, 40%, 50%, 60%, 70%, 80%, 90%, 95% or 99% or more as compared to the expression of a target gene or the activity or level of the protein encoded by a target gene which has not been targeted by an RNA interfering agent.

“Short interfering RNA” (siRNA), also referred to herein as “small interfering RNA” is defined as an agent which functions to inhibit expression of a target gene, e.g., by RNAi. An siRNA can be chemically synthesized, can be produced by in vitro transcription, or can be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, 22, or 23 nucleotides in length, and can contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

siRNAs also include small hairpin (also called stem loop) RNAs (shRNAs). In one embodiment, these shRNAs are composed of a short (e.g., about 19 to about 25 nucleotide) antisense strand, followed by a nucleotide loop of about 5 to about 9 nucleotides, and the analogous sense strand. Alternatively, the sense strand can precede the nucleotide loop structure and the antisense strand can follow. These shRNAs can be contained in plasmids, retroviruses, and lentiviruses and expressed from, for example, the pol III U6 promoter, or another promoter (see, e.g., Stewart, et al. (2003) RNA April; 9(4):493-501, incorporated by reference herein in its entirety).

The target gene or sequence of the RNA interfering agent can be a cellular gene or genomic sequence, e.g. the MT1 and/or MT2 gene sequence. A siRNA can be substantially homologous to the target gene or genomic sequence, or a fragment thereof. As used in this context, the term “homologous” is defined as being substantially identical, sufficiently complementary, or similar to the target mRNA, or a fragment thereof, to effect RNA interference of the target. In addition to native RNA molecules, RNA suitable for inhibiting or interfering with the expression of a target sequence include RNA derivatives and analogs. Preferably, the siRNA is identical to its target sequence.

The siRNA preferably targets only one sequence. Each of the RNA interfering agents, such as siRNAs, can be screened for potential off-target effects by, for example, expression profiling. Such methods are known to one skilled in the art and are described, for example, in Jackson et al, Nature Biotechnology 6:635-637, 2003. In addition to expression profiling, one can also screen the potential target sequences for similar sequences in the sequence databases to identify potential sequences which can have off-target effects. For example, according to Jackson et al. (Id.) 15, or perhaps as few as 11 contiguous nucleotides of sequence identity are sufficient to direct silencing of non-targeted transcripts. Therefore, one can initially screen the proposed siRNAs to avoid potential off-target silencing using the sequence identity analysis by any known sequence comparison methods, such as BLAST.

siRNA molecules need not be limited to those molecules containing only RNA, but, for example, further encompasses chemically modified nucleotides and non-nucleotides, and also include molecules wherein a ribose sugar molecule is substituted for another sugar molecule or a molecule which performs a similar function. Moreover, a non-natural linkage between nucleotide residues can be used, such as a phosphorothioate linkage. For example, siRNA containing D-arabinofuranosyl structures in place of the naturally-occurring D-ribonucleosides found in RNA can be used in RNAi molecules according to the present invention (U.S. Pat. No. 5,177,196). Other examples include RNA molecules containing the o-linkage between the sugar and the heterocyclic base of the nucleoside, which confers nuclease resistance and tight complementary strand binding to the oligonucleotides molecules similar to the oligonucleotides containing 2′-O-methyl ribose, arabinose and particularly D-arabinose (U.S. Pat. No. 5,177,196).

The RNA strand can be derivatized with a reactive functional group of a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatized with a variety of groups.

Other useful RNA derivatives incorporate nucleotides having modified carbohydrate moieties, such as 2′O-alkylated residues or 2′-O-methyl ribosyl derivatives and 2′-O-fluoro ribosyl derivatives. The RNA bases can also be modified. Any modified base useful for inhibiting or interfering with the expression of a target sequence can be used. For example, halogenated bases, such as 5-bromouracil and 5-iodouracil can be incorporated. The bases can also be alkylated, for example, 7-methylguanosine can be incorporated in place of a guanosine residue. Non-natural bases that yield successful inhibition can also be incorporated.

The most preferred siRNA modifications include 2′-deoxy-2′-fluorouridine or locked nucleic acid (LNA) nucleotides and RNA duplexes containing either phosphodiester or varying numbers of phosphorothioate linkages. Such modifications are known to one skilled in the art and are described, for example, in Braasch et al., Biochemistry, 42: 7967-7975, 2003. Most of the useful modifications to the siRNA molecules can be introduced using chemistries established for antisense oligonucleotide technology. Preferably, the modifications involve minimal 2′-O-methyl modification, preferably excluding such modification. Modifications also preferably exclude modifications of the free 5′-hydroxyl groups of the siRNA.

siRNA and miRNA molecules having various “tails” covalently attached to either their 3′- or to their 5′-ends, or to both, are also known in the art and can be used to stabilize the siRNA and miRNA molecules delivered using the methods of the present invention. Generally speaking, intercalating groups, various kinds of reporter groups and lipophilic groups attached to the 3′ or 5′ ends of the RNA molecules are well known to one skilled in the art and are useful according to the methods of the present invention. Descriptions of syntheses of 3′-cholesterol or 3′-acridine modified oligonucleotides applicable to preparation of modified RNA molecules useful according to the present invention can be found, for example, in the articles: Gamper, H. B., Reed, M. W., Cox, T., Virosco, J. S., Adams, A. D., Gall, A., Scholler, J. K., and Meyer, R. B. (1993) Facile Preparation and Exonuclease Stability of 3′-Modified Oligodeoxynucleotides. Nucleic Acids Res. 21 145-150; and Reed, M. W., Adams, A. D., Nelson, J. S., and Meyer, R. B., Jr. (1991) Acridine and Cholesterol-Derivatized Solid Supports for Improved Synthesis of 3′-Modified Oligonucleotides. Bioconjugate Chem. 2 217-225 (1993).

Other siRNAs useful for targeting the MT1 and/or MT2 gene can be readily designed and tested. Accordingly, siRNAs useful for the methods described herein include siRNA molecules of about 15 to about 40 or about 15 to about 28 nucleotides in length, which are homologous to the IGPR-1 gene. In some embodiments, a MT1 and/or MT2 targeting siRNA molecules have a length of about 25 to about 29 nucleotides. In some embodiments, a MT1 and/or MT2 targeting siRNA molecules have a length of about 27, 28, 29, or 30 nucleotides. In some embodiments, a MT1 and/or MT2 targeting siRNA molecules can also comprise a 3′ hydroxyl group. In some embodiments, a MT1 and/or MT2 targeting siRNA molecules can be single-stranded or double stranded; such molecules can be blunt ended or comprise overhanging ends (e.g., 5′, 3′). In specific embodiments, the RNA molecule can be a double stranded and either blunt ended or comprises overhanging ends.

In one embodiment, at least one strand of a MT1 and/or MT2 targeting RNA molecule has a 3′ overhang from about 0 to about 6 nucleotides (e.g., pyrimidine nucleotides, purine nucleotides) in length. In other embodiments, the 3′ overhang is from about 1 to about 5 nucleotides, from about 1 to about 3 nucleotides and from about 2 to about 4 nucleotides in length. In one embodiment the IGPR-1 targeting RNA molecule is double stranded—one strand has a 3′ overhang and the other strand can be blunt-ended or have an overhang. In the embodiment in which a MT1 and/or MT2 targeting RNA molecule is double stranded and both strands comprise an overhang, the length of the overhangs can be the same or different for each strand. In a particular embodiment, the RNA of the present invention comprises about 19, 20, 21, or 22 nucleotides which are paired and which have overhangs of from about 1 to about 3, particularly about 2, nucleotides on both 3′ ends of the RNA. In one embodiment, the 3′ overhangs can be stabilized against degradation. In a preferred embodiment, the RNA is stabilized by including purine nucleotides, such as adenosine or guanosine nucleotides. Alternatively, substitution of pyrimidine nucleotides by modified analogues, e.g., substitution of uridine 2 nucleotide 3′ overhangs by 2′-deoxythymidine is tolerated and does not affect the efficiency of RNAi. The absence of a 2′ hydroxyl significantly enhances the nuclease resistance of the overhang in tissue culture medium.

In some embodiments, where gene silencing RNAi of MT1 and/or MT2 are not commercially available, gene silencing RNAi agents targeting inhibition of MT1 and/or MT2 can be produced by one of ordinary skill in the art and according to the methods as disclosed herein. In some embodiments, the assessment of the expression and/or knock down of MT1 and/or MT2 mRNA and/or protein can be determined using commercially available kits known by persons of ordinary skill in the art. Others can be readily prepared by those of skill in the art based on the known sequence of the target mRNA.

In some embodiments, an inhibitor of MT1 and/or MT2 is a gene silencing RNAi agent which downregulates or decreases MT1 and/or MT2 mRNA levels is a 25-nt hairpin sequence. In some embodiments, an inhibitor of MT1 and/or MT2 is a gene silencing RNAi, such as, for example, a shRNA sequence.

In one embodiment, the RNA interfering agents used in the methods described herein are taken up actively by cells in vivo following intravenous injection, e.g., hydrodynamic injection, without the use of a vector, illustrating efficient in vivo delivery of the RNA interfering agents, e.g., the siRNAs used in the methods of the invention.

Other strategies for delivery of the RNA interfering agents, e.g., the siRNAs or shRNAs used in the methods of the invention, can also be employed, such as, for example, delivery by a vector, e.g., a plasmid or viral vector, e.g., a lentiviral vector. Such vectors can be used as described, for example, in Xiao-Feng Qin et al. Proc. Natl. Acad. Sci. U.S.A., 100: 183-188. Other delivery methods include delivery of the RNA interfering agents, e.g., the siRNAs or shRNAs of the invention, using a basic peptide by conjugating or mixing the RNA interfering agent with a basic peptide, e.g., a fragment of a TAT peptide, mixing with cationic lipids or formulating into particles.

As noted, the dsRNA, such as siRNA or shRNA can be delivered using an inducible vector, such as a tetracycline inducible vector. Methods described, for example, in Wang et al. Proc. Natl. Acad. Sci. 100: 5103-5106, using pTet-On vectors (BD Biosciences Clontech, Palo Alto, Calif.) can be used. In some embodiments, a vector can be a plasmid vector, a viral vector, or any other suitable vehicle adapted for the insertion and foreign sequence and for the introduction into eukaryotic cells. The vector can be an expression vector capable of directing the transcription of the DNA sequence of the agonist or antagonist nucleic acid molecules into RNA. Viral expression vectors can be selected from a group comprising, for example, reteroviruses, lentiviruses, Epstein Barr virus-, bovine papilloma virus, adenovirus- and adeno-associated-based vectors or hybrid virus of any of the above. In one embodiment, the vector is episomal. The use of a suitable episomal vector provides a means of maintaining the antagonist nucleic acid molecule in the subject in high copy number extra chromosomal DNA thereby eliminating potential effects of chromosomal integration.

RNA interference molecules and nucleic acid inhibitors useful in the methods as disclosed herein can be produced using any known techniques such as direct chemical synthesis, through processing of longer double stranded RNAs by exposure to recombinant Dicer protein or Drosophila embryo lysates, through an in vitro system derived from S2 cells, using phage RNA polymerase, RNA-dependent RNA polymerase, and DNA based vectors. Use of cell lysates or in vitro processing can further involve the subsequent isolation of the short, for example, about 21-23 nucleotide, siRNAs from the lysate, etc. Chemical synthesis usually proceeds by making two single stranded RNA-oligomers followed by the annealing of the two single stranded oligomers into a double stranded RNA. Other examples include methods disclosed in WO 99/32619 and WO 01/68836 that teach chemical and enzymatic synthesis of siRNA. Moreover, numerous commercial services are available for designing and manufacturing specific siRNAs (see, e.g., QIAGEN Inc., Valencia, Calif. and AMBION Inc., Austin, Tex.).

The terms “antimir” “microRNA inhibitor” or “miR inhibitor” are synonymous and refer to oligonucleotides that interfere with the activity of specific miRNAs Inhibitors can adopt a variety of configurations including single stranded, double stranded (RNA/RNA or RNA/DNA duplexes), and hairpin designs, in general, microRNA inhibitors comprise one or more sequences or portions of sequences that are complementary or partially complementary with the mature strand (or strands) of the miRNA to be targeted, in addition, the miRNA inhibitor can also comprise additional sequences located 5′ and 3′ to the sequence that is the reverse complement of the mature miRNA. The additional sequences can be the reverse complements of the sequences that are adjacent to the mature miRNA in the pri-miRNA from which the mature miRNA is derived, or the additional sequences can be arbitrary sequences (having a mixture of A, G, C, U, or dT). In some embodiments, one or both of the additional sequences are arbitrary sequences capable of forming hairpins. Thus, in some embodiments, the sequence that is the reverse complement of the miRNA is flanked on the 5′ side and on the 3′ side by hairpin structures. MicroRNA inhibitors, when double stranded, can include mismatches between nucleotides on opposite strands.

In some embodiments, an agent is protein or polypeptide or RNAi agent which inhibits the expression of the MT1 and/or MT2 gene. In such embodiments cells can be modified (e.g., by homologous recombination) to provide increased expression of such an agent, for example by replacing, in whole or in part, the naturally occurring promoter with all or part of a heterologous promoter so that the cells express an inhibitor of MT1 and/or MT2, for example a protein or RNAi agent (e.g. gene silencing-RNAi agent). Typically, a heterologous promoter is inserted in such a manner that it is operatively linked to the desired nucleic acid encoding the agent. See, for example, PCT International Publication No. WO 94/12650 by Transkaryotic Therapies, Inc., PCT International Publication No. WO 92/20808 by Cell Genesys, Inc., and PCT International Publication No. WO 91/09955 by Applied Research Systems. Cells also can be engineered to express an endogenous gene comprising the inhibitor agent under the control of inducible regulatory elements, in which case the regulatory sequences of the endogenous gene can be replaced by homologous recombination. Gene activation techniques are described in U.S. Pat. No. 5,272,071 to Chappel; U.S. Pat. No. 5,578,461 to Sherwin et al.; PCT/US92/09627 (WO93/09222) by Selden et al.; and PCT/US90/06436 (WO91/06667) by Skoultchi et al. The agent can be prepared by culturing transformed host cells under culture conditions suitable to express the miRNA. The resulting expressed agent can then be purified from such culture (i.e., from culture medium or cell extracts) using known purification processes, such as gel filtration and ion exchange chromatography. The purification of the peptide or nucleic acid agent inhibitor of MT1 and/or MT2 can also include an affinity column containing agents which will bind to the protein; one or more column steps over such affinity resins as concanavalin A-agarose, heparin-Toyopearl™ or Cibacrom blue 3GA Sepharose; one or more steps involving hydrophobic interaction chromatography using such resins as phenyl ether, butyl ether, or propyl ether; immunoaffinity chromatography, or complementary cDNA affinity chromatography.

In one embodiment, a nucleic acid inhibitor of MT1 and/or MT2, e.g. (gene silencing RNAi agent) can be obtained synthetically, for example, by chemically synthesizing a nucleic acid by any method of synthesis known to the skilled artisan. A synthesized nucleic acid inhibitor of IGPR-1 can then be purified by any method known in the art. Methods for chemical synthesis of nucleic acids include, but are not limited to, in vitro chemical synthesis using phosphotriester, phosphate or phosphoramidite chemistry and solid phase techniques, or via deoxynucleoside H-phosphonate intermediates (see U.S. Pat. No. 5,705,629 to Bhongle).

In some circumstances, for example, where increased nuclease stability of a nucleic acid inhibitor is desired, nucleic acids having nucleic acid analogs and/or modified internucleoside linkages can be used. Nucleic acids containing modified internucleoside linkages can also be synthesized using reagents and methods that are well known in the art. For example, methods of synthesizing nucleic acids containing phosphonate phosphorothioate, phosphorodithioate, phosphoramidate methoxyethyl phosphoramidate, formacetal, thioformacetal, diisopropylsilyl, acetamidate, carbamate, dimethylene-sulfide (—CH₂—S—CH₂), dimethylene-sulfoxide (—CH₂—SO—CH₂), dimethylene-sulfone (—CH₂—SO₂—CH₂), 2′-O-alkyl, and 2′-deoxy-2′-fluoro′phosphorothioate internucleoside linkages are well known in the art (see Uhlmann et al., 1990, Chem. Rev. 90:543-584; Schneider et al., 1990, Tetrahedron Lett. 31:335 and references cited therein). U.S. Pat. Nos. 5,614,617 and 5,223,618 to Cook, et al., 5,714, 606 to Acevedo, et al, U.S. Pat. No. 5,378,825 to Cook, et al., U.S. Pat. Nos. 5,672,697 and 5,466,786 to Buhr, et al., U.S. Pat. No. 5,777,092 to Cook, et al., U.S. Pat. No. 5,602,240 to De Mesmacker, et al., U.S. Pat. No. 5,610,289 to Cook, et al. and U.S. Pat. No. 5,858,988 to Wang, also describe nucleic acid analogs for enhanced nuclease stability and cellular uptake.

Synthetic siRNA molecules, including shRNA molecules, can also easily be obtained using a number of techniques known to those of skill in the art. For example, the siRNA molecule can be chemically synthesized or recombinantly produced using methods known in the art, such as using appropriately protected ribonucleoside phosphoramidites and a conventional DNA/RNA synthesizer (see, e.g., Elbashir, S. M. et al. (2001) Nature 411:494-498; Elbashir, S. M., W. Lendeckel and T. Tuschl (2001) Genes & Development 15:188-200; Harborth, J. et al. (2001) J. Cell Science 114:4557-4565; Masters, J. R. et al. (2001) Proc. Natl. Acad. Sci., USA 98:8012-8017; and Tuschl, T. et al. (1999) Genes & Development 13:3191-3197). Alternatively, several commercial RNA synthesis suppliers are available including, but are not limited to, Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). As such, siRNA molecules are not overly difficult to synthesize and are readily provided in a quality suitable for RNAi. In addition, dsRNAs can be expressed as stem loop structures encoded by plasmid vectors, retroviruses and lentiviruses (Paddison, P. J. et al. (2002) Genes Dev. 16:948-958; McManus, M. T. et al. (2002) RNA 8:842-850; Paul, C. P. et al. (2002) Nat. Biotechnol. 20:505-508; Miyagishi, M. et al. (2002) Nat. Biotechnol. 20:497-500; Sui, G. et al. (2002) Proc. Natl. Acad. Sci., USA 99:5515-5520; Brummelkamp, T. et al. (2002) Cancer Cell 2:243; Lee, N. S., et al. (2002) Nat. Biotechnol. 20:500-505; Yu, J. Y., et al. (2002) Proc. Natl. Acad. Sci., USA 99:6047-6052; Zeng, Y., et al. (2002) Mol. Cell 9:1327-1333; Rubinson, D. A., et al. (2003) Nat. Genet. 33:401-406; Stewart, S. A., et al. (2003) RNA 9:493-501). These vectors generally have a polIII promoter upstream of the dsRNA and can express sense and antisense RNA strands separately and/or as a hairpin structures. Within cells, Dicer processes the short hairpin RNA (shRNA) into effective siRNA.

In some embodiments, an inhibitor of MT1 and/or MT2 is a gene silencing siRNA molecule which targets a MT1 and/or MT2 gene and targets the coding mRNA sequence of MT1 and/or MT2, beginning from about 25 to 50 nucleotides, from about 50 to 75 nucleotides, or from about 75 to 100 nucleotides downstream of the start codon. One method of designing a siRNA molecule of the present invention involves identifying the 29 nucleotide sequence motif AA(N29)TT (where N can be any nucleotide), and selecting hits with at least 25%, 30%, 35%, 40%, 45%, 50%, 55%, 60%, 65%, 70% or 75% G/C content. The “TT” portion of the sequence is optional. Alternatively, if no such sequence is found, the search can be extended using the motif NA(N21), where N can be any nucleotide. In this situation, the 3′ end of the sense siRNA can be converted to TT to allow for the generation of a symmetric duplex with respect to the sequence composition of the sense and antisense 3′ overhangs. The antisense siRNA molecule can then be synthesized as the complement to nucleotide positions 1 to 21 of the 23 nucleotide sequence motif. The use of symmetric 3′ TT overhangs can be advantageous to ensure that the small interfering ribonucleoprotein particles (siRNPs) are formed with approximately equal ratios of sense and antisense target RNA-cleaving siRNPs (Elbashir et al. (2001) supra and Elbashir et al. 2001 supra). Analysis of sequence databases, including but not limited to the NCBI, BLAST, Derwent and GenSeq as well as commercially available oligosynthesis software such as Oligoengine®, can also be used to select siRNA sequences against EST libraries to ensure that only one gene is targeted.

siRNAs useful for the methods described herein include siRNA molecules of about 15 to about 40 or about 15 to about 28 nucleotides in length, which are homologous to MT1 and/or MT2 isoforms. Preferably, a targeting siRNA molecule to the IGPR-1 gene has a length of about 19 to about 25 nucleotides. More preferably, the targeting siRNA molecules have a length of about 19, 20, 21, or 22 nucleotides. The targeting siRNA molecules can also comprise a 3′ hydroxyl group. The targeting siRNA molecules can be single-stranded or double stranded; such molecules can be blunt ended or comprise overhanging ends (e.g., 5′, 3′). In specific embodiments, the RNA molecule is double stranded and either blunt ended or comprises overhanging ends.

In one embodiment, at least one strand of a MT1 and/or MT2 RNAi targeting RNA molecule has a 3′ overhang from about 0 to about 6 nucleotides (e.g., pyrimidine nucleotides, purine nucleotides) in length. In other embodiments, the 3′ overhang is from about 1 to about 5 nucleotides, from about 1 to about 3 nucleotides and from about 2 to about 4 nucleotides in length. In one embodiment the targeting RNA molecule is double stranded—one strand has a 3′ overhang and the other strand can be blunt-ended or have an overhang. In the embodiment in which the targeting RNA molecule is double stranded and both strands comprise an overhang, the length of the overhangs can be the same or different for each strand. In a particular embodiment, the RNA of the present invention comprises about 19, 20, 21, or 22 nucleotides which are paired and which have overhangs of from about 1 to about 3, particularly about 2, nucleotides on both 3′ ends of the RNA. In one embodiment, the 3′ overhangs can be stabilized against degradation. In a preferred embodiment, the RNA is stabilized by including purine nucleotides, such as adenosine or guanosine nucleotides. Alternatively, substitution of pyrimidine nucleotides by modified analogues, e.g., substitution of uridine 2 nucleotide 3′ overhangs by 2′-deoxythymidine is tolerated and does not affect the efficiency of RNAi. The absence of a 2′ hydroxyl significantly enhances the nuclease resistance of the overhang in tissue culture medium.

Oligonucleotide Modifications

Unmodified oligonucleotides can be less than optimal in some applications, e.g., unmodified oligonucleotides can be prone to degradation by e.g., cellular nucleases. Nucleases can hydrolyze nucleic acid phosphodiester bonds. However, chemical modifications to one or more of the subunits of oligonucleotide can confer improved properties, and, e.g., can render oligonucleotides more stable to nucleases.

Modified nucleic acids and nucleotide surrogates can include one or more of: (i) alteration, e.g., replacement, of one or both of the non-linking phosphate oxygens and/or of one or more of the linking phosphate oxygens in the phosphodiester backbone linkage. (ii) alteration, e.g., replacement, of a constituent of the ribose sugar, e.g., of the 2′ hydroxyl on the ribose sugar; (iii) wholesale replacement of the phosphate moiety with “dephospho” linkers; (iv) modification or replacement of a naturally occurring base with a non-natural base; (v) replacement or modification of the ribose-phosphate backbone; (vi) modification of the 3′ end or 5′ end of the oligonucleotide, e.g., removal, modification or replacement of a terminal phosphate group or conjugation of a moiety, e.g., a fluorescently labeled moiety, to either the 3′ or 5′ end of oligonucleotide; and (vii) modification of the sugar (e.g., six membered rings).

The terms replacement, modification, alteration, and the like, as used in this context, do not imply any process limitation, e.g., modification does not mean that one must start with a reference or naturally occurring ribonucleic acid and modify it to produce a modified ribonucleic acid bur rather modified simply indicates a difference from a naturally occurring molecule.

As oligonucleotides are polymers of subunits or monomers, many of the modifications described herein can occur at a position which is repeated within an oligonucleotide, e.g., a modification of a nucleobase, a sugar, a phosphate moiety, or the non-bridging oxygen of a phosphate moiety. It is not necessary for all positions in a given oligonucleotide to be uniformly modified, and in fact more than one of the aforementioned modifications can be incorporated in a single oligonucleotide or even at a single nucleoside within an oligonucleotide.

In some cases the modification will occur at all of the subject positions in the oligonucleotide but in many, and in fact in most cases it will not. By way of example, a modification can only occur at a 3′ or 5′ terminal position, can only occur in the internal region, can only occur in a terminal regions, e.g. at a position on a terminal nucleotide or in the last 2, 3, 4, 5, or 10 nucleotides of an oligonucleotide. A modification can occur in a double strand region, a single strand region, or in both. A modification can occur only in the double strand region of an oligonucleotide or can only occur in a single strand region of an oligonucleotide. E.g., a phosphorothioate modification at a non-bridging oxygen position can only occur at one or both termini, can only occur in a terminal regions, e.g., at a position on a terminal nucleotide or in the last 2, 3, 4, 5, or 10 nucleotides of a strand, or can occur in double strand and single strand regions, particularly at termini. The 5′ end or ends can be phosphorylated.

A modification described herein can be the sole modification, or the sole type of modification included on multiple nucleotides, or a modification can be combined with one or more other modifications described herein. The modifications described herein can also be combined onto an oligonucleotide, e.g. different nucleotides of an oligonucleotide have different modifications described herein.

In some embodiments it is particularly preferred, e.g., to enhance stability, to include particular nucleobases in overhangs, or to include modified nucleotides or nucleotide surrogates, in single strand overhangs, e.g., in a 5′ or 3′ overhang, or in both. E.g., it can be desirable to include purine nucleotides in overhangs. In some embodiments all or some of the bases in a 3′ or 5′ overhang will be modified, e.g., with a modification described herein. Modifications can include, e.g., the use of modifications at the 2′ OH group of the ribose sugar, e.g., the use of deoxyribonucleotides, e.g., deoxythymidine, instead of ribonucleotides, and modifications in the phosphate group, e.g., phosphothioate modifications. Overhangs need not be homologous with the target sequence.

Specific Modifications to Oligonucleotide

The Phosphate Group

The phosphate group is a negatively charged species. The charge is distributed equally over the two non-bridging oxygen atoms. However, the phosphate group can be modified by replacing one of the oxygens with a different substituent. One result of this modification to RNA phosphate backbones can be increased resistance of the oligoribonucleotide to nucleolytic breakdown. Thus while not wishing to be bound by theory, it can be desirable in some embodiments to introduce alterations which result in either an uncharged linker or a charged linker with unsymmetrical charge distribution.

Examples of modified phosphate groups include phosphorothioate, phosphoroselenates, borano phosphates, borano phosphate esters, hydrogen phosphonates, phosphoroamidates, alkyl or aryl phosphonates and phosphotriesters. In certain embodiments, one of the non-bridging phosphate oxygen atoms in the phosphate backbone moiety can be replaced by any of the following: S, Se, BR₃ (R is hydrogen, alkyl, aryl), C (i.e. an alkyl group, an aryl group, etc. . . . ), H, NR₂ (R is hydrogen, alkyl, aryl), or OR (R is alkyl or aryl). The phosphorous atom in an unmodified phosphate group is achiral. However, replacement of one of the non-bridging oxygens with one of the above atoms or groups of atoms renders the phosphorous atom chiral; in other words a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligoribonucleotides diastereomers. Thus, while not wishing to be bound by theory, modifications to both non-bridging oxygens, which eliminate the chiral center, e.g. phosphorodithioate formation, can be desirable in that they cannot produce diastereomer mixtures. Thus, the non-bridging oxygens can be independently any one of S, Se, B, C, H, N, or OR (R is alkyl or aryl).

The phosphate linker can also be modified by replacement of bridging oxygen, (i.e. oxygen that links the phosphate to the nucleoside), with nitrogen (bridged phosphoroamidates), sulfur (bridged phosphorothioates) and carbon (bridged methylenephosphonates). The replacement can occur at the either linking oxygen or at both the linking oxygens. When the bridging oxygen is the 3′-oxygen of a nucleoside, replacement with carbon is preferred. When the bridging oxygen is the 5′-oxygen of a nucleoside, replacement with nitrogen is preferred.

Replacement of the Phosphate Group

The phosphate group can be replaced by non-phosphorus containing connectors. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino Preferred replacements include the methylenecarbonylamino and methylenemethylimino groups.

Modified phosphate linkages where at least one of the oxygens linked to the phosphate has been replaced or the phosphate group has been replaced by a non-phosphorous group, are also referred to as “non-phosphodiester backbone linkage.”

Replacement of Ribophosphate Backbone

Oligonucleotide-mimicking scaffolds can also be constructed wherein the phosphate linker and ribose sugar are replaced by nuclease resistant nucleoside or nucleotide surrogates. While not wishing to be bound by theory, it is believed that the absence of a repetitively charged backbone diminishes binding to proteins that recognize polyanions (e.g. nucleases). Again, while not wishing to be bound by theory, it can be desirable in some embodiment, to introduce alterations in which the bases are tethered by a neutral surrogate backbone. Examples include the morpholino, cyclobutyl, pyrrolidine and peptide nucleic acid (PNA) nucleoside surrogates. A preferred surrogate is a PNA surrogate.

Sugar Modifications

An oligonucleotide can include modification of all or some of the sugar groups of the nucleic acid. E.g., the 2′ hydroxyl group (OH) can be modified or replaced with a number of different “oxy” or “deoxy” substituents. While not being bound by theory, enhanced stability is expected since the hydroxyl can no longer be deprotonated to form a 2′-alkoxide ion. The 2′-alkoxide can catalyze degradation by intramolecular nucleophilic attack on the linker phosphorus atom. Again, while not wishing to be bound by theory, it can be desirable to some embodiments to introduce alterations in which alkoxide formation at the 2′ position is not possible.

Examples of “oxy”-2′ hydroxyl group modifications include alkoxy or aryloxy (OR, e.g., R ═H, alkyl, cycloalkyl, aryl, aralkyl, heteroaryl or sugar); polyethyleneglycols (PEG), O(CH₂CH₂O)ₙCH₂CH₂OR; “locked” nucleic acids (LNA) in which the 2′ hydroxyl is connected, e.g., by a methylene bridge, to the 4′ carbon of the same ribose sugar; O-AMINE (AMINE=NH₂; alkylamino, dialkylamino, heterocyclyl, arylamino, diaryl amino, heteroaryl amino, or diheteroaryl amino, ethylene diamine, polyamino) and aminoalkoxy, O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino) It is noteworthy that oligonucleotides containing only the methoxyethyl group (MOE), (OCH₂CH₂OCH₃, a PEG derivative), exhibit nuclease stabilities comparable to those modified with the robust phosphorothioate modification.

“Deoxy” modifications include hydrogen (i.e. deoxyribose sugars, which are of particular relevance to the overhang portions of partially ds RNA); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino), —NHC(O)R (R=alkyl, cycloalkyl, aryl, aralkyl, heteroaryl or sugar), cyano; mercapto; alkyl-thio-alkyl; thioalkoxy; thioalkyl; and alkyl, cycloalkyl, aryl, alkenyl and alkynyl, which can be optionally substituted with e.g., an amino functionality.

The sugar group can also contain one or more carbons that possess the opposite stereochemical configuration than that of the corresponding carbon in ribose. Thus, an oligonucleotide can include nucleotides containing e.g., arabinose, as the sugar. The monomer can have an alpha linkage at the 1′ position on the sugar, e.g., alpha-nucleosides. Oligonucleotides can also include “abasic” sugars, which lack a nucleobase at C-1′. These abasic sugars can also be further containing modifications at one or more of the constituent sugar atoms. Oligonucleotides can also contain one or more sugars that are in the L form, e.g. L-nucleosides.

Preferred substituents are 2′-O-Me (2′-O-methyl), 2′-O-MOE (2′-O-methoxyethyl), 2′-F, 2′-O-[2-(methylamino)-2-oxoethyl] (2′-O-NMA), 2′-S-methyl, 2′-O—CH2-(4′-C) (LNA), 2′-O—CH₂CH₂-(4′-C) (ENA), 2′-O-aminopropyl (2′-O-AP), 2′-O-dimethylaminoethyl (2′-O-DMAOE), 2′-O-dimethylaminopropyl (2′-O-DMAP) and 2′-O-dimethylaminoethyloxyethyl (2′-O-DMAEOE).

Terminal Modifications

The 3-prime (3′) and 5-prime (5′) ends of an oligonucleotide can be modified. Such modifications can be at the 3′ end, 5′ end or both ends of the molecule. They can include modification or replacement of an entire terminal phosphate or of one or more of the atoms of the phosphate group. E.g., the 3′ and 5′ ends of an oligonucleotide can be conjugated to other functional molecular entities such as labeling moieties, e.g., fluorophores (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a linker. The terminal atom of the linker can connect to or replace the linking atom of the phosphate group or the C-3′ or C-5′ O, N, S or C group of the sugar. Alternatively, the linker can connect to or replace the terminal atom of a nucleotide surrogate (e.g., PNAs).

When a linker/phosphate-functional molecular entity-linker/phosphate array is interposed between two strands of a dsRNA, this array can substitute for a hairpin RNA loop in a hairpin-type RNA agent.

Terminal modifications useful for modulating activity include modification of the 5′ end with phosphate or phosphate analogs. E.g., in preferred embodiments antisense strands of dsRNAs, are 5′ phosphorylated or include a phosphoryl analog at the 5′ prime terminus. 5′-phosphate modifications include those which are compatible with RISC mediated gene silencing. Modifications at the 5′-terminal end can also be useful in stimulating or inhibiting the immune system of a subject. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g. 5′-alpha-thiotriphosphate, 5′-beta-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (R=alkyl=methyl, ethyl, isopropyl, propyl, etc., e.g. RP(OH)(O)—O-5′-, (OH)₂(O)P-5′-CH₂—), 5′-alkyletherphosphonates (R=alkylether=methoxymethyl (MeOCH₂—), ethoxymethyl, etc., e.g. RP(OH)(O)—O-5′-). Other embodiments include replacement of oxygen/sulfur with BH₃, BH₃— and/or Se.

Terminal modifications can also be useful for monitoring distribution, and in such cases the preferred groups to be added include fluorophores, e.g., fluorscein or an ALEXA® dye, e.g., ALEXA® 488. Terminal modifications can also be useful for enhancing uptake, useful modifications for this include cholesterol. Terminal modifications can also be useful for cross-linking an RNA agent to another moiety; modifications useful for this include mitomycin C.

Nucleobases

Adenine, guanine, cytosine and uracil are the most common bases found in RNA. These bases can be modified or replaced to provide RNA's having improved properties. For example, nuclease resistant oligoribonucleotides can be prepared with these bases or with synthetic and natural nucleobases (e.g., inosine, thymine, xanthine, hypoxanthine, nubularine, isoguanisine, or tubercidine) and any one of the above modifications. Alternatively, substituted or modified analogs of any of the above bases and “universal bases” can be employed. Examples include 2-(halo)adenine, 2-(alkyl)adenine, 2-(propyl)adenine, 2 (amino)adenine, 2-(aminoalkyll)adenine, 2 (aminopropyl)adenine, 2 (methylthio) N⁶ (isopentenyl)adenine, 6 (alkyl)adenine, 6 (methyl)adenine, 7 (deaza)adenine, 8 (alkenyl)adenine, 8-(alkyl)adenine, 8 (alkynyl)adenine, 8 (amino)adenine, 8-(halo)adenine, 8-(hydroxyl)adenine, 8 (thioalkyl)adenine, 8-(thiol)adenine, N⁶-(isopentyl)adenine, N⁶ (methyl)adenine, N⁶,N⁶ (dimethyl)adenine, 2-(alkyl)guanine, 2 (propyl)guanine, 6-(alkyl)guanine, 6 (methyl)guanine, 7 (alkyl)guanine, 7 (methyl)guanine, 7 (deaza)guanine, 8 (alkyl)guanine, 8-(alkenyl)guanine, 8 (alkynyl)guanine, 8-(amino)guanine, 8 (halo)guanine, 8-(hydroxyl)guanine, 8 (thioalkyl)guanine, 8-(thiol)guanine, N (methyl)guanine, 2-(thio)cytosine, 3 (deaza) 5 (aza)cytosine, 3-(alkyl)cytosine, 3 (methyl)cytosine, 5-(alkyl)cytosine, 5-(alkynyl)cytosine, 5 (halo)cytosine, 5 (methyl)cytosine, 5 (propynyl)cytosine, 5 (propynyl)cytosine, 5 (trifluoromethyl)cytosine, 6-(azo)cytosine, N4 (acetyl)cytosine, 3 (3 amino-3 carboxypropyl)uracil, 2-(thio)uracil, 5 (methyl) 2 (thio)uracil, 5 (methylaminomethyl)-2 (thio)uracil, 4-(thio)uracil, 5 (methyl) 4 (thio)uracil, 5 (methylaminomethyl)-4 (thio)uracil, 5 (methyl) 2,4 (dithio)uracil, 5 (methylaminomethyl)-2,4 (dithio)uracil, 5 (2-aminopropyl)uracil, 5-(alkyl)uracil, 5-(alkynyl)uracil, 5-(allylamino)uracil, 5 (aminoallyl)uracil, 5 (aminoalkyl)uracil, 5 (guanidiniumalkyl)uracil, 5 (1,3-diazole-1-alkyl)uracil, 5-(cyanoalkyl)uracil, 5-(dialkylaminoalkyl)uracil, 5 (dimethylaminoalkyl)uracil, 5-(halo)uracil, 5-(methoxy)uracil, uracil-5 oxyacetic acid, 5 (methoxycarbonylmethyl)-2-(thio)uracil, 5 (methoxycarbonyl-methyl)uracil, 5 (propynyl)uracil, 5 (propynyl)uracil, 5 (trifluoromethyl)uracil, 6 (azo)uracil, dihydrouracil, N3 (methyl)uracil, 5-uracil (i.e., pseudouracil), 2 (thio)pseudouracil, 4 (thio)pseudouracil, 2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4 (thio)pseudouracil, 5-(methyl)-4 (thio)pseudouracil, 5-(alkyl)-2,4 (dithio)pseudouracil, 5-(methyl)-2,4 (dithio)pseudouracil, 1 substituted pseudouracil, 1 substituted 2(thio)-pseudouracil, 1 substituted 4 (thio)pseudouracil, 1 substituted 2,4-(dithio)pseudouracil, 1 (aminocarbonylethylenyl)-pseudouracil, 1 (aminocarbonylethylenyl)-2(thio)-pseudouracil, 1 (aminocarbonylethylenyl)-4 (thio)pseudouracil, 1 (aminocarbonylethylenyl)-2,4-(dithio)pseudouracil, 1 (aminoalkylaminocarbonylethylenyl)-pseudouracil, 1 (aminoalkylamino-carbonylethylenyl)-2(thio)-pseudouracil, 1 (aminoalkylaminocarbonylethylenyl)-4 (thio)pseudouracil, 1 (aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5 nitroindole, 3 nitropyrrole, 6-(aza)pyrimidine, 2 (amino)purine, 2,6-(diamino)purine, 5 substituted pyrimidines, N²-substituted purines, N⁶-substituted purines, O⁶-substituted purines, substituted 1,2,4-triazoles, or any O-alkylated or N-alkylated derivatives thereof;

Further purines and pyrimidines include those disclosed in U.S. Pat. No. 3,687,808, hereby incorporated by reference, those disclosed in the Concise Encyclopedia Of Polymer Science And Engineering, pages 858-859, Kroschwitz, J. I., ed. John Wiley & Sons, 1990, and those disclosed by Englisch et al., Angewandte Chemie, International Edition, 1991, 30, 613.

Cationic Groups

Modifications to oligonucleotides can also include attachment of one or more cationic groups to the sugar, base, and/or the phosphorus atom of a phosphate or modified phosphate backbone moiety. A cationic group can be attached to any atom capable of substitution on a natural, unusual or universal base. A preferred position is one that does not interfere with hybridization, i.e., does not interfere with the hydrogen bonding interactions needed for base pairing. A cationic group can be attached e.g., through the C2′ position of a sugar or analogous position in a cyclic or acyclic sugar surrogate. Cationic groups can include e.g., protonated amino groups, derived from e.g., O-AMINE (AMINE=NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); or NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino).

Placement within an Oligonucleotide

Some modifications can preferably be included on an oligonucleotide at a particular location, e.g., at an internal position of a strand, or on the 5′ or 3′ end of an oligonucleotide. A preferred location of a modification on an oligonucleotide, can confer preferred properties on the agent. For example, preferred locations of particular modifications can confer optimum gene silencing properties, or increased resistance to endonuclease or exonuclease activity.

One or more nucleotides of an oligonucleotide can have a 2′-5′ linkage. One or more nucleotides of an oligonucleotide can have inverted linkages, e.g. 3′-3′, 5′-5′, 2′-2′ or 2′-3′ linkages.

An oligonucleotide can comprise at least one 5′-pyrimidine-purine-3′ (5′-PyPu-3′) dinucleotide wherein the pyrimidine is modified with a modification chosen independently from a group consisting of 2′-O-Me (2′-O-methyl), 2′-O-MOE (2′-O-methoxyethyl), 2′-F, 2′-O-[2-(methylamino)-2-oxoethyl] (2′-O-NMA), 2′-S-methyl, 2′-O—CH₂-(4′-C) (LNA) and 2′-O—CH₂CH₂-(4′-C) (ENA).

In one embodiment, the 5′-most pyrimidines in all occurrences of sequence motif 5′-pyrimidine-purine-3′ (5′-PyPu-3′) dinucleotide in the oligonucleotide are modified with a modification chosen from a group consisting of 2″-O-Me (2′-O-methyl), 2′-O-MOE (2′-O-methoxyethyl), 2′-F, 2′-O-[2-(methylamino)-2-oxoethyl] (2′-O-NMA), 2′-S-methyl, 2′-O—CH₂-(4′-C) (LNA) and 2′-O—CH₂CH₂-(4′-C) (ENA).

A double-stranded oligonucleotide can include at least one 5′-uridine-adenine-3′ (5′-UA-3′) dinucleotide wherein the uridine is a 2′-modified nucleotide, or a 5′-uridine-guanine-3′ (5′-UG-3′) dinucleotide, wherein the 5′-uridine is a 2′-modified nucleotide, or a terminal 5′-cytidine-adenine-3′ (5′-CA-3′) dinucleotide, wherein the 5′-cytidine is a 2′-modified nucleotide, or a terminal 5′-uridine-uridine-3′ (5′-UU-3′) dinucleotide, wherein the 5′-uridine is a 2′-modified nucleotide, or a terminal 5′-cytidine-cytidine-3′ (5′-CC-3′) dinucleotide, wherein the 5′-cytidine is a 2′-modified nucleotide, or a terminal 5′-cytidine-uridine-3′ (5′-CU-3′) dinucleotide, wherein the 5′-cytidine is a 2′-modified nucleotide, or a terminal 5′-uridine-cytidine-3′ (5′-UC-3′) dinucleotide, wherein the 5′-uridine is a 2′-modified nucleotide. Double-stranded oligonucleotides including these modifications are particularly stabilized against endonuclease activity.

General References

The oligoribonucleotides and oligoribonucleotides used in accordance with this invention can be synthesized with solid phase synthesis, see for example “Oligonucleotide synthesis, a practical approach”, Ed. M. J. Gait, IRL Press, 1984; “Oligonucleotides and Analogues, A Practical Approach”, Ed. F. Eckstein, IRL Press, 1991 (especially Chapter 1, Modern machine-aided methods of oligodeoxyribonucleotide synthesis, Chapter 2, Oligoribonucleotide synthesis, Chapter 3, 2′-O-Methyloligoribonucleotide-s: synthesis and applications, Chapter 4, Phosphorothioate oligonucleotides, Chapter 5, Synthesis of oligonucleotide phosphorodithioates, Chapter 6, Synthesis of oligo-2′-deoxyribonucleoside methylphosphonates, and. Chapter 7, Oligodeoxynucleotides containing modified bases. Other particularly useful synthetic procedures, reagents, blocking groups and reaction conditions are described in Martin, P., Helv. Chim. Acta, 1995, 78, 486-504; Beaucage, S. L. and Iyer, R. P., Tetrahedron, 1992, 48, 2223-2311 and Beaucage, S. L. and Iyer, R. P., Tetrahedron, 1993, 49, 6123-6194, or references referred to therein. Modification described in WO 00/44895, WO01/75164, or WO02/44321 can be used herein. The disclosure of all publications, patents, and published patent applications listed herein are hereby incorporated by reference.

Phosphate Group References

The preparation of phosphinate oligoribonucleotides is described in U.S. Pat. No. 5,508,270. The preparation of alkyl phosphonate oligoribonucleotides is described in U.S. Pat. No. 4,469,863. The preparation of phosphoramidite oligoribonucleotides is described in U.S. Pat. No. 5,256,775 or U.S. Pat. No. 5,366,878. The preparation of phosphotriester oligoribonucleotides is described in U.S. Pat. No. 5,023,243. The preparation of borano phosphate oligoribonucleotide is described in U.S. Pat. Nos. 5,130,302 and 5,177,198. The preparation of 3′-Deoxy-3′-amino phosphoramidate oligoribonucleotides is described in U.S. Pat. No. 5,476,925. 3′-Deoxy-3′-methylenephosphonate oligoribonucleotides is described in An, H, et al. J. Org. Chem. 2001, 66, 2789-2801. Preparation of sulfur bridged nucleotides is described in Sproat et al. Nucleosides Nucleotides 1988, 7,651 and Crosstick et al. Tetrahedron Left. 1989, 30, 4693.

Sugar Group References

Modifications to the 2′ modifications can be found in Verma, S. et al. Annu. Rev. Biochem. 1998, 67, 99-134 and all references therein. Specific modifications to the ribose can be found in the following references: 2′-fluoro (Kawasaki et. al., J. Med. Chem., 1993, 36, 831-841), 2′-MOE (Martin, P. Helv. Chim. Acta 1996, 79, 1930-1938), “LNA” (Wengel, J. Acc. Chem. Res. 1999, 32, 301-310).

Replacement of the Phosphate Group References

Methylenemethylimino linked oligoribonucleotides, also identified herein as MMI linked oligoribonucleotides, methylenedimethylhydrazo linked oligoribonucleotides, also identified herein as MDH linked oligoribonucleotides, and methylenecarbonylamino linked oligonucleosides, also identified herein as amide-3 linked oligoribonucleotides, and methyleneaminocarbonyl linked oligonucleosides, also identified herein as amide-4 linked oligoribonucleotides as well as mixed backbone compounds having, as for instance, alternating MMI and PO or PS linkages can be prepared as is described in U.S. Pat. Nos. 5,378,825, 5,386,023, 5,489,677 and in published PCT applications PCT/US92/04294 and PCT/US92/04305 (published as WO 92/20822 WO and 92/20823, respectively). Formacetal and thioformacetal linked oligoribonucleotides can be prepared as is described in U.S. Pat. Nos. 5,264,562 and 5,264,564. Ethylene oxide linked oligoribonucleotides can be prepared as is described in U.S. Pat. No. 5,223,618. Siloxane replacements are described in Cormier, J. F. et al. Nucleic Acids Res. 1988, 16, 4583. Carbonate replacements are described in Tittensor, J. R. J. Chem. Soc. C 1971, 1933. Carboxymethyl replacements are described in Edge, M. D. et al. J. Chem. Soc. Perkin Trans. 1 1972, 1991. Carbamate replacements are described in Stirchak, E. P. Nucleic Acids Res. 1989, 17, 6129.

Replacement of the Phosphate-Ribose Backbone References

Cyclobutyl sugar surrogate compounds can be prepared as is described in U.S. Pat. No. 5,359,044. Pyrrolidine sugar surrogate can be prepared as is described in U.S. Pat. No. 5,519,134. Morpholino sugar surrogates can be prepared as is described in U.S. Pat. Nos. 5,142,047 and 5,235,033, and other related patent disclosures. Peptide Nucleic Acids (PNAs) are known per se and can be prepared in accordance with any of the various procedures referred to in Peptide Nucleic Acids (PNA): Synthesis, Properties and Potential Applications, Bioorganic & Medicinal Chemistry, 1996, 4, 5-23. They can also be prepared in accordance with U.S. Pat. No. 5,539,083 which is incorporated herein in its entirety by reference.

Terminal Modification References.

Terminal modifications are described in Manoharan, M. et al. Antisense and Nucleic Acid Drug Development 12, 103-128 (2002) and references therein.

Nuclebases References

N-2 substituted purine nucleoside amidites can be prepared as is described in U.S. Pat. No. 5,459,255. 3-Deaza purine nucleoside amidites can be prepared as is described in U.S. Pat. No. 5,457,191. 5,6-Substituted pyrimidine nucleoside amidites can be prepared as is described in U.S. Pat. No. 5,614,617. 5-Propynyl pyrimidine nucleoside amidites can be prepared as is described in U.S. Pat. No. 5,484,908. Additional references are disclosed in the above section on base modifications

Oligonucleotide Production

The oligonucleotide compounds of the invention can be prepared using solution-phase or solid-phase organic synthesis. Organic synthesis offers the advantage that the oligonucleotide strands comprising non-natural or modified nucleotides can be easily prepared. Any other means for such synthesis known in the art can additionally or alternatively be employed. It is also known to use similar techniques to prepare other oligonucleotides, such as the phosphorothioates, phosphorodithioates and alkylated derivatives. The double-stranded oligonucleotide compounds of the invention can be prepared using a two-step procedure. First, the individual strands of the double-stranded molecule are prepared separately. Then, the component strands are annealed.

Regardless of the method of synthesis, the oligonucleotide can be prepared in a solution (e.g., an aqueous and/or organic solution) that is appropriate for formulation. For example, the oligonucleotide preparation can be precipitated and redissolved in pure double-distilled water, and lyophilized. The dried oligonucleotide can then be resuspended in a solution appropriate for the intended formulation process.

Teachings regarding the synthesis of particular modified oligonucleotides can be found in the following U.S. patents or pending patent applications: U.S. Pat. Nos. 5,138,045 and 5,218,105, drawn to polyamine conjugated oligonucleotides; U.S. Pat. No. 5,212,295, drawn to monomers for the preparation of oligonucleotides having chiral phosphorus linkages; U.S. Pat. Nos. 5,378,825 and 5,541,307, drawn to oligonucleotides having modified backbones; U.S. Pat. No. 5,386,023, drawn to backbone-modified oligonucleotides and the preparation thereof through reductive coupling; U.S. Pat. No. 5,457,191, drawn to modified nucleobases based on the 3-deazapurine ring system and methods of synthesis thereof; U.S. Pat. No. 5,459,255, drawn to modified nucleobases based on N-2 substituted purines; U.S. Pat. No. 5,521,302, drawn to processes for preparing oligonucleotides having chiral phosphorus linkages; U.S. Pat. No. 5,539,082, drawn to peptide nucleic acids; U.S. Pat. No. 5,554,746, drawn to oligonucleotides having β-lactam backbones; U.S. Pat. No. 5,571,902, drawn to methods and materials for the synthesis of oligonucleotides; U.S. Pat. No. 5,578,718, drawn to nucleosides having alkylthio groups, wherein such groups can be used as linkers to other moieties attached at any of a variety of positions of the nucleoside; U.S. Pat. Nos. 5,587,361 and 5,599,797, drawn to oligonucleotides having phosphorothioate linkages of high chiral purity; U.S. Pat. No. 5,506,351, drawn to processes for the preparation of 2′-O-alkyl guanosine and related compounds, including 2,6-diaminopurine compounds; U.S. Pat. No. 5,587,469, drawn to oligonucleotides having N-2 substituted purines; U.S. Pat. No. 5,587,470, drawn to oligonucleotides having 3-deazapurines; U.S. Pat. No. 5,223,168, and U.S. Pat. No. 5,608,046, both drawn to conjugated 4′-desmethyl nucleoside analogs; U.S. Pat. Nos. 5,602,240, and 5,610,289, drawn to backbone-modified oligonucleotide analogs; and U.S. Pat. Nos. 6,262,241, and 5,459,255, drawn to, inter alia, methods of synthesizing 2′-fluoro-oligonucleotides.

Delivery of MT1 and/or MT2 RNA Interfering Agents:

Methods of delivering RNAi agents, e.g., an siRNA, or vectors containing an RNAi agent, to the target cells (e.g., basal cells or cells of the lung and/or respiratory system or other desired target cells) are well known to persons of ordinary skill in the art. In some embodiments, a RNAi agent (e.g. gene silencing-RNAi agent) which is an inhibitor of IGPR-1 can be administered to a subject via aerosol means, for example using a nebulizer and the like. In alternative embodiments, administration of a RNAi agent (e.g. gene silencing-RNAi agent) which is an inhibitor of IGPR-1 can include, for example (i) injection of a composition containing the RNA interfering agent, e.g., an siRNA, or (ii) directly contacting the cell, e.g., a cell of the respiratory system, with a composition comprising an RNAi agent, e.g., an siRNA. In another embodiment, RNAi agents, e.g., an siRNA can be injected directly into any blood vessel, such as vein, artery, venule or arteriole, via, e.g., hydrodynamic injection or catheterization. In some embodiments an RNAi inhibitor of IGPR-1 can delivered to specific organs, for example the liver, bone marrow or systemic administration.

Administration can be by a single injection or by two or more injections. In some embodiments, a RNAi agent is delivered in a pharmaceutically acceptable carrier. One or more RNAi agents can be used simultaneously, e.g. one or more gene silencing RNAi agent inhibitors of MT1 and/or MT2 can be together. The RNA interfering agents, e.g., the siRNA inhibitors of MT1 and/or MT2, can be delivered singly, or in combination with other RNA interfering agents, e.g., siRNAs, such as, for example siRNAs directed to other cellular genes. A gene silencing-RNAi agent inhibitor of MT1 and/or MT2 can also be administered in combination with other pharmaceutical agents which are used to treat or prevent neurodegenerative diseases or disorders.

In one embodiment, specific cells are targeted with RNA interference, limiting potential side effects of RNA interference caused by non-specific targeting of RNA interference. The method can use, for example, a complex or a fusion molecule comprising a cell targeting moiety and an RNA interference binding moiety that is used to deliver RNAi effectively into cells. For example, an antibody-protamine fusion protein when mixed with an siRNA, binds siRNA and selectively delivers the siRNA into cells expressing an antigen recognized by the antibody, resulting in silencing of gene expression only in those cells that express the antigen which is identified by the antibody. In some embodiments, the antibody can be any antibody which identifies an antigen expressed on cells expressing MT1 and/or MT2. In some embodiments, the antibody is an antibody which binds to the MT1 and/or MT2 antigen, but where the antibody can or does not inhibit MT1 and/or MT2 function. In some embodiments, the siRNA can be conjugated to a MT1 and/or MT2 antagonist, for example where the MT1 and/or MT2 antagonist is a polypeptide, and where the conjugation with the RNAi does not interrupt the function of the MT1 and/or MT2 antagonist.

In some embodiments, a siRNA or RNAi binding moiety is a protein or a nucleic acid binding domain or fragment of a protein, and the binding moiety is fused to a portion of the targeting moiety. The location of the targeting moiety can be either in the carboxyl-terminal or amino-terminal end of the construct or in the middle of the fusion protein.

In some embodiments, a viral-mediated delivery mechanism can also be employed to deliver siRNAs, e.g. siRNAs (e.g. gene silencing RNAi agents) inhibitors of MT1 and/or MT2, to cells in vitro and in vivo as described in Xia, H. et al. (2002) Nat Biotechnol 20(10):1006). Plasmid- or viral-mediated delivery mechanisms of shRNA can also be employed to deliver shRNAs to cells in vitro and in vivo as described in Rubinson, D. A., et al. ((2003) Nat. Genet. 33:401-406) and Stewart, S. A., et al. ((2003) RNA 9:493-501). Alternatively, in other embodiments, a RNAi agent, e.g., a gene silencing-RNAi agent inhibitor of MT1 and/or MT2 can also be introduced into cells via the vascular or extravascular circulation, the blood or lymph system, and the cerebrospinal fluid.

In general, any method of delivering a nucleic acid molecule can be adapted for use with an RNAi interference molecule (see e.g., Akhtar S. and Julian R L. (1992) Trends Cell. Biol. 2(5):139-144; WO94/02595, which are incorporated herein by reference in their entirety). However, there are three factors that are important to consider in order to successfully deliver an RNAi molecule in vivo: (a) biological stability of the RNAi molecule, (2) preventing non-specific effects, and (3) accumulation of the RNAi molecule in the target tissue. The non-specific effects of an RNAi molecule can be minimized by local administration by e.g., direct injection into a tissue including, for example, a tumor or topically administering the molecule.

Local administration of an RNAi molecule to a treatment site limits the exposure of the e.g., siRNA to systemic tissues and permits a lower dose of the RNAi molecule to be administered. Several studies have shown successful knockdown of gene products when an RNAi molecule is administered locally. For example, intraocular delivery of a VEGF siRNA by intravitreal injection in cynomolgus monkeys (Tolentino, M J., et al (2004) Retina 24:132-138) and subretinal injections in mice (Reich, S J., et al (2003) Mol. Vis. 9:210-216) were both shown to prevent neovascularization in an experimental model of age-related macular degeneration. In addition, direct intratumoral injection of an siRNA in mice reduces tumor volume (Pille, J., et al (2005) Mol. Ther. 11:267-274) and can prolong survival of tumor-bearing mice (Kim, W J., et al (2006) Mol. Ther. 14:343-350; Li, S., et al (2007) Mol. Ther. 15:515-523). RNA interference has also shown success with local delivery to the CNS by direct injection (Dorn, G., et al (2004) Nucleic Acids 32:e49; Tan, P H., et al (2005) Gene Ther. 12:59-66; Makimura, H., et al (2002) BMC Neurosci. 3:18; Shishkina, G T., et al (2004) Neuroscience 129:521-528; Thakker, E R., et al (2004) Proc. Natl. Acad. Sci. U.S.A. 101:17270-17275; Akaneya, Y., et al (2005) J. Neurophysiol. 93:594-602) and to the lungs by intranasal administration (Howard, K A., et al (2006) Mol. Ther. 14:476-484; Zhang, X., et al (2004) J. Biol. Chem. 279:10677-10684; Bitko, V., et al (2005) Nat. Med. 11:50-55).

For administering an RNAi molecule systemically for the treatment of a disease, the RNAi molecule can be either be modified or alternatively delivered using a drug delivery system; both methods act to prevent the rapid degradation of the RNAi molecule by endo- and exo-nucleases in vivo. Modification of the RNAi molecule or the pharmaceutical carrier can also permit targeting of the RNAi molecule to the target tissue and avoid undesirable off-target effects.

RNA interference molecules can be modified by chemical conjugation to lipophilic groups such as cholesterol to enhance cellular uptake and prevent degradation. For example, an siRNA directed against ApoB conjugated to a lipophilic cholesterol moiety was injected systemically into mice and resulted in knockdown of apoB mRNA in both the liver and jejunum (Soutschek, J., et al (2004) Nature 432:173-178). Conjugation of an RNAi molecule to an aptamer has been shown to inhibit tumor growth and mediate tumor regression in a mouse model of prostate cancer (McNamara, J O., et al (2006) Nat. Biotechnol. 24:1005-1015).

In an alternative embodiment, the RNAi molecules can be delivered using drug delivery systems such as e.g., a nanoparticle, a dendrimer, a polymer, liposomes, or a cationic delivery system. Positively charged cationic delivery systems facilitate binding of an RNA interference molecule (negatively charged) and also enhance interactions at the negatively charged cell membrane to permit efficient uptake of an siRNA by the cell. Cationic lipids, dendrimers, or polymers can either be bound to an RNA interference molecule, or induced to form a vesicle or micelle (see e.g., Kim S H., et al (2008) Journal of Controlled Release 129(2):107-116) that encases an RNAi molecule. The formation of vesicles or micelles further prevents degradation of the RNAi molecule when administered systemically. Methods for making and administering cationic-RNAi complexes are well within the abilities of one skilled in the art (see e.g., Sorensen, D R., et al (2003) J. Mol. Biol 327:761-766; Verma, U N., et al (2003) Clin. Cancer Res. 9:1291-1300; Arnold, A S et al (2007) J. Hypertens. 25:197-205, which are incorporated herein by reference in their entirety).

Some non-limiting examples of drug delivery systems useful for systemic administration of RNAi include DOTAP (Sorensen, D R., et al (2003), supra; Verma, U N., et al (2003), supra), Oligofectamine, “solid nucleic acid lipid particles” (Zimmermann, T S., et al (2006) Nature 441:111-114), cardiolipin (Chien, P Y., et al (2005) Cancer Gene Ther. 12:321-328; Pal, A., et al (2005) Int J. Oncol. 26:1087-1091), polyethyleneimine (Bonnet M E., et al (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A., et al (2007) Biochem. Soc. Trans. 35:61-67; Yoo, H., et al (1999) Pharm. Res. 16:1799-1804). In some embodiments, an RNAi molecule forms a complex with cyclodextrin for systemic administration. Methods for administration and pharmaceutical compositions of RNAi molecules and cyclodextrins can be found in U.S. Pat. No. 7,427,605, which is herein incorporated by reference in its entirety. Specific methods for administering an RNAi molecule for the inhibition of angiogenesis can be found in e.g., U.S. Patent Application No. 20080152654, which is herein incorporated by reference in its entirety.

In some embodiments, the siRNA, dsRNA, or shRNA vector can be administered systemically, such as intravenously, e. g. via central venous catheter (CVC or central venous line or central venous access catheter) placed into a large vein in the neck (internal jugular vein), chest (subclavian vein) or groin (femoral vein). Methods of systemic delivery of siRNA, dsRNA, or shRNA vector are well known in the art, e. g. as described herein and in Gao and Huang, 2008, (Mol. Pharmaceutics, Web publication December 30) and review by Rossi, 2006, Gene Therapy, 13:583-584. The siRNA, dsRNA, or shRNA vector can be formulated in various ways, e. g. conjugation of a cholesterol moiety to one of the strands of the siRNA duplex for systemic delivery to the liver and jejunum (Soutschek J. et. al. 2004, Nature, 432:173-178), complexing of siRNAs to protamine fused with an antibody fragment for receptor-mediated targeting of siRNAs (Song E, et al. 2005, Nat Biotechnol., 23: 709-717) and the use of a lipid bilayer system by Morrissey et al. 2005 (Nat Biotechnol., 23: 1002-1007). The lipid bilayer system produces biopolymers that are in the 120 nanometer diameter size range, and are labeled as SNALPs, for Stable-Nucleic-Acid-Lipid-Particles. The lipid combination protects the siRNAs from serum nucleases and allows cellular endosomal uptake and subsequent cytoplasmic release of the siRNAs (see WO/2006/007712). These references are incorporated by reference in their entirety.

The dose of the particular RNAi agent will be in an amount necessary to effect RNA interference, e.g., gene silencing of the MT1 and/or MT2 gene, thereby leading to decrease in IGPR-1 gene expression level and subsequent decrease in the MT1 and/or MT2 protein level.

It is also known that RNAi molecules do not have to match perfectly to their target sequence. Preferably, however, the 5′ and middle part of the antisense (guide) strand of the siRNA is perfectly complementary to the target nucleic acid sequence of a the MT1 and/or MT2 genes as disclosed herein.

Accordingly, the RNAi molecules functioning as gene silencing-RNAi agents inhibitors of MT1 and/or MT2 as disclosed herein are for example, but are not limited to, unmodified and modified double stranded (ds) RNA molecules including short-temporal RNA (stRNA), small interfering RNA (siRNA), short-hairpin RNA (shRNA), microRNA (miRNA), double-stranded RNA (dsRNA), (see, e.g. Baulcombe, Science 297:2002-2003, 2002). The dsRNA molecules, e.g. siRNA, also can contain 3′ overhangs, preferably 3′UU or 3′TT overhangs. In one embodiment, the siRNA molecules of the present invention do not include RNA molecules that comprise ssRNA greater than about 30-40 bases, about 40-50 bases, about 50 bases or more. In one embodiment, the siRNA molecules of the present invention are double stranded for more than about 25%, more than about 50%, more than about 60%, more than about 70%, more than about 80%, more than about 90% of their length.

In some embodiments, a gene silencing RNAi nucleic acid inhibitors of IGPR-1 is any agent which binds to and inhibits the expression of the MT1 and/or MT2gene, where the expression of the MT1 and/or MT2 gene is inhibited.

In another embodiment of the invention, agents which are inhibitors of MT1 and/or MT2 are catalytic nucleic acid constructs, such as, for example ribozymes, which are capable of cleaving RNA transcripts and thereby preventing the production of wildtype protein. Ribozymes are targeted to and anneal with a particular sequence by virtue of two regions of sequence complementary to the target flanking the ribozyme catalytic site. After binding, the ribozyme cleaves the target in a site specific manner. The design and testing of ribozymes which specifically recognize and cleave sequences of the gene products described herein, for example for cleavage of the MT1 and/or MT2 proteins or MT1 and/or MT2 genes can be achieved by techniques well known to those skilled in the art (for example Lleber and Strauss, (1995) Mol Cell Biol 15:540.551, the disclosure of which is incorporated herein by reference).

Proteins and Peptide Inhibitors of MT1 and/or MT2.

In some embodiments, an inhibitor of MT1 and/or MT2 is a protein and/or peptide inhibitor, for example, but are not limited to mutated proteins; therapeutic proteins and recombinant proteins of MT1 and/or MT2 as well as dominant negative inhibitors (e.g., C-terminal fragments of MT1 and/or MT2) as disclosed herein. Proteins and peptides inhibitors can also include for example mutated proteins, genetically modified proteins, peptides, synthetic peptides, recombinant proteins, chimeric proteins, antibodies, humanized proteins, humanized antibodies, chimeric antibodies, modified proteins and fragments thereof.

In some embodiments, an inhibitor of MT1 and/or MT2 is a dominant negative variants of the MT1 and/or MT2 protein, for example a non-functional variant of the MT1 and/or MT2 protein, e.g., a fragment of at least about 50, or at least about 40, or at least about 30, or at least about 20 or at least about 10 or more than 10 amino acids of the sequences of MT1 and/or MT2 as disclosed herein.

Methods to Detect Agents as Modulators of MT1 and/or MT2

As discussed herein, one aspect of the present invention relates to methods and compositions comprising inhibitors of MTs (e g., inhibitors of MT1 and/or MT2 isoforms) to increase IL-10 production from Tr1 cells, for example, for the treatment of autoimmune diseases and treatment of grafts-versus-hosts disease and to prevent transplant rejection in a transplant recipient subject. In some embodiments, such inhibitors of MTs (e g., inhibitors of MT1 and/or MT2 isoforms) is used to contact Tr1 cells obtained from the subject ex vivo, and the MT inhibitor-treated Tr1 cells are administered to a subject in need thereof, e.g., a subject with an autoimmune disease and/or a subject who is a transplant recipient.

In another aspect of the present invention, inhibitors of MTs (e g., inhibitors of MT1 and/or MT2 isoforms) can be used to reduce CD8+ T cell exhaustion, for example in diseases of chronic immune diseases and/or cancer. In some embodiments, such inhibitors of MTs (e g., inhibitors of MT1 and/or MT2 isoforms) is used to contact CD8+ T-cells obtained from the subject ex vivo, and the MT inhibitor-treated CD8+ T-cells are administered to a subject in need thereof, e.g., a subject with chronic immune condition and/or cancer.

In alternative embodiments, an activator of MTs (e.g., an overexpression and/or exogenous MT1 and/or MT2) can be used to reduce the differentiation of Tr1 cells from CD4+ cells, and/or reduced proliferation of Tr1 cells, and/or decrease IL-10 production, for example for use in method to treat a subject having excess or high IL-10 levels, for example, during chronic immune conditions. In some embodiments, such a MT activator is used to contact Tr1 cells obtained from the subject ex vivo, and the MT activator-treated Tr1 cells are administered to a subject in need thereof, e.g., where it is desirable to reduce IL-10.

Modulation of T cell tolerance can also be measured by determining the proliferation of T cells in the presence of a relevant antigen assayed, e.g. by a ³H-thymidine incorporation assay or cell number. Markers of T cell activation after exposure to the relevant antigen can also be assayed, e.g. flow cytometry analysis of cell surface markers indicative of T cell activation (e.g. CD69, CD30, CD25, and HLA-DR). Reduced T cell activation in response to antigen-challenge is indicative of tolerance induction. Conversely, increased T cell activation in response to antigen-challenge is indicative of reduced tolerance.

Modulation of T cell tolerance can also be measured, in some embodiments, by determining the degree to which the modulating agent inhibits or increase the activity of its target. For example, the SEB model can be used to measure T cell tolerance and modulation thereof. In normal mice, neonatal injection of staphylococcal enterotoxin B (SEB) induces tolerance in T cells that express reactive T cell receptor (TCR) V beta regions. If, in the presence of an MT modulating agent, T cells expressing reactive TCR V beta regions (e.g., Vbeta8) display a statistically significant reduction or increase in T cell activity than T cells not contacted with the modulating agent, the modulating agent is one that modulates T cell tolerance.

Other in vivo models of peripheral tolerance that can be used in some aspects and embodiments to measure modulation in T cell tolerance using the modulating agents described herein include, for example, models for peripheral tolerance in which homogeneous populations of T cells from TCR transgenic and double transgenic mice are transferred into hosts that constitutively express the antigen recognized by the transferred T cells, e.g., the H-Y antigen TCR transgenic; pigeon cytochrome C antigen TCR transgenic; or hemagglutinin (HA) TCR transgenic. In such models, T cells expressing the TCR specific for the antigen constitutively or inducibly expressed by the recipient mice typically undergo an immediate expansion and proliferative phase, followed by a period of unresponsiveness, which is reversed when the antigen is removed and/or antigen expression is inhibited. Accordingly, if, in the presence of an MT inhibitors (e.g., MT1 and/or MT2 inhibitor), for example, in such models if the T cells proliferate or expand, show cytokine activity (e.g., increased IL-10 production), etc. significantly more than T cells in the absence of the inhibitory agent, than that agent is one that reduces T cell tolerance. Such measurements of proliferation can occur in vivo using T cells labeled with BrDU, CFSE or another intravital dye that allows tracking of proliferation prior to transferring to a recipient animal expressing the antigen, or cytokine reporter T cells, or using ex vivo methods to analyze cellular proliferation and/or cytokine production, such as thymidine proliferation assays, ELISA, cytokine bead assays, and the like.

Modulation of T cell tolerance can also be assessed by examination of tumor infiltrating lymphocytes or T lymphocytes within lymph nodes that drain from an established tumor. Such T cells exhibit features of “exhaustion” through expression of cell surface molecules, such asTIM-3, for example, and decreased secretion of cytokines such as interferon-γ. Accordingly, if, in the presence of an inhibitory agent, increased quantities of T cells with, for example, 1) antigen specificity for tumor associated antigens are observed (e.g. as determined by major histocompatibility complex class I or class II tetramers which contain tumor associated peptides) and/or 2) that are capable of secreting high levels of interferon-γ and cytolytic effector molecules such as granzyme-B, relative to that observed in the absence of the inhibitory agent, this would be evidence that T cell tolerance had been reduced.

### MT Activators

Also provided herein, in other aspects, are compositions comprising at least one MT activator (e.g., MT1 and/or MT2 isoform activator) or agonists for use in decreasing IL-10 production from Tr1 cells. For example, in some embodiments, an activator of MTs (e.g., an overexpression and/or exogenous MT1 and/or MT2) can be used to reduce the differentiation of Tr1 cells from CD4+ cells, and/or reduce the proliferation of Tr1 cells, and/or decrease IL-10 production from Tr1 cells, for example, for use in a method to treat a subject having excess or high IL-10 levels, for example, during chronic immune conditions. In some embodiments, such a MT activator is used to contact Tr1 cells obtained from the subject ex vivo, and the MT activator-treated Tr1 cells are administered to a subject in need thereof, e.g., where it is desirable to reduce IL-10.

As used herein, the terms “MT activator,” “MT agonist,” MT activator agent,” and “MT agonist agent” refer to a molecule or agent that mimics or up-regulates (e.g., increases, potentiates or supplements) the expression and/or biological activity of any one MT1 and/or MT2 isoform in vitro, in situ, and/or in vivo, including, downstream pathways mediated by MT1 and/or MT2 signaling, such as, for example, decreased IL-10 induction, and/or decreased cellular response to IL-27 and/or MT1 and/or MT2. A MT activator (e.g., MT1 and/or MT2 isoform activator) or agonist can be a wild-type MT1 and/or MT2 protein or derivative thereof having at least one bioactivity of the wild-type MT1 and/or mT2 isoform. A MT activator (e.g., MT1 and/or MT2 isoform activator) or agonist can also be a compound that up-regulates expression of a MT1 and/or MT2 isoform or its subunits. A MT activator (e.g., MT1 and/or MT2 isoform activator) or agonist can also be a compound which increases the interaction of a MT with its ligand, e.g., metal for example, zinc. Exemplary MT activators (e.g., MT1 and/or MT2 isoform activator) or agonists contemplated for use in the various aspects and embodiments described herein include, but are not limited to, anti-MT1 and/or MT2 antibodies or antigen-binding fragments thereof that specifically bind to MT1 and/or MT2; RNA or DNA aptamers that bind to the MT1 and/or MT2 and mimic metal binding to MT1 and/or MT2; MT1 and/or MT2 structural analogs or soluble MT1 and/or MT2 mimics or fusion polypeptides thereof; and small molecule agents that target or bind to MT1 and/or MT2 and act as functional mimics. In some embodiments of these aspects and all such aspects described herein, an MT activator (e.g., MT1 and/or MT2 isoform activator) or agonist (e.g., an antibody or antigen-binding fragment thereof) selectively binds (physically interacts with) binds to an isoform of MT1 and/or MT2, and increases (activates/enhances) MT1 and/or MT2 downstream signaling, and/or increases or up-regulates a MT1 and/or MT2 isoform synthesis, production or release. In some embodiments of these aspects and all such aspects described herein, an MT activator (e.g., MT1 and/or MT2 isoform activator) or agonist increases or enhances expression (i.e., transcription or translation) of any MT, e.g., any isoform of MT1 and/or MT2.

As used herein, a MT activator (e.g., MT1 and/or MT2 isoform) agonist has the ability to increase or enhance the activity and/or expression of MT1 and/or MT2 in a cell (e.g., T cells, such as CD8+ or CD4+ T cells) by at least 5%, at least 10%, at least 20%, at least 30%, at least 40%, at least 50%, at least 60%, at least 70%, at least 80%, at least 90%, at least 95%, at least 98%, at least 99%, at least 100%, at least 1.5-fold, at least 2-fold, at least 5-fold, at least 10-fold, at least 25-fold, at least 50-fold, at least 100-fold, at least 1000-fold, or more relative to the activity or expression level in the absence of the IL-27 activator or agonist.

In some embodiments of the compositions and methods described herein, a MT activator (e.g., MT1 and/or MT2 isoform) or agonist increases or enhances MT1 and/or MT2 mediated signal transduction. In some embodiments of the compositions and methods described herein, a MT activator (e.g., MT1 and/or MT2 isoform) or agonist increases or enhances MT1 and/or MT2-mediated transcription factor induction or activation, for example, an increase in STAT1 and/or STAT3 phosphorylation. In some embodiments of the compositions and methods described herein, a MT activator (e.g., MT1 and/or MT2 isoform) or agonist decreases IL-27-induced IL-10 production.

In some embodiments of the compositions and methods described herein, a MT activator (e.g., MT1 and/or MT2 isoform) or agonist is an antibody or antigen-binding fragment thereof that selectively binds or physically interacts with an isoform of MT1 and/or MT2 subunit and enhances or increases the activity of MT1 and/or MT2.

In some embodiments of the compositions and methods described herein, the binding sites of a MT activator (e.g., MT1 and/or MT2 isoform) or agonists, such as an antibody or antigen-binding fragment thereof, are directed against a metal binding site. By binding to a metal ligand interaction site, a MT activator (e.g., MT1 and/or MT2 isoform) or agonist described herein can mimic or recapitulate metal to the MT and increase the activity or expression of MT1 and/or MT2, and downstream MT1 and/or MT2 signaling consequences (e.g., decreased IL-10 induction, and/or suppression of a cellular response to IL-27).

In some embodiments of the compositions and methods described herein, a MT activator (e.g., MT1 and/or MT2 isoform) or agonist is an antibody or antigen-binding fragment thereof that binds or physically interacts with any isoform of MT1 and/or MT2. In some embodiments of the compositions and methods described herein, a MT activator (e.g., MT1 and/or MT2 isoform) activator or agonist is an antibody or antigen-binding fragment thereof that binds a MT1 and/or MT2 isoform and increases and/or promotes its biological activity. Exemplary assays to measure increases or up-regulation of downstream MT1 and/or MT2 signaling pathway activities are known to those of ordinary skill in the art and are provided herein in the Examples.

In some embodiments of the compositions and methods described herein, a MT activator (e.g., MT1 and/or MT2 isoform) or agonist is a monoclonal antibody. In some embodiments of the compositions and methods described herein, a MT activator (e.g., MT1 and/or MT2 isoform) or agonist is an antibody fragment or antigen-binding fragment, as described in more detail elsewhere herein.

In some embodiments of the compositions and methods described herein, a MT activator (e.g., MT1 and/or MT2 isoform) or agonist is a chimeric antibody derivative of the MT1 and/or MT2 agonist antibodies and antigen-binding fragments thereof, as described in more detail elsewhere herein.

In some embodiments of the compositions and methods described herein, a MT activator (e.g., MT1 and/or MT2 isoform) or agonist is a humanized antibody derivative, as described in more detail elsewhere herein.

In some embodiments, a MT activator (e.g., MT1 and/or MT2 isoform) or agonist antibodies and antigen-binding fragments thereof described herein, i.e., antibodies that are useful for increasing T cell exhaustion, include derivatives that are modified, i.e., by the covalent attachment of any type of molecule to the antibody.

A MT activator (e.g., MT1 and/or MT2 isoform) or agonist antibodies and antigen-binding fragments thereof described herein for use in increasing or promoting T cell exhaustion, as well as any of the other antibodies or antigen-binding fragments thereof described herein in various aspects and embodiments, can be generated by any suitable method known in the art.

In some embodiments, a MT activator (e.g., MT1 and/or MT2 isoform) or agonist antibodies and antigen-binding fragments thereof described herein, i.e., antibodies that are useful for increasing T cell exhaustion, are completely human antibodies or antigen-binding fragments thereof, which are particularly desirable for the therapeutic treatment of human patients. Human antibodies can be made by a variety of methods known in the art, and as described in more detail elsewhere herein.

In some embodiments of the compositions and methods described herein, a MT activator (e.g., MT1 and/or MT2 isoform) or agonist is a small molecule activator or agonist, including, but is not limited to, small peptides or peptide-like molecules, soluble peptides, and synthetic non-peptidyl organic or inorganic compounds. A small molecule activator or agonist can have a molecular weight of any of about 100 to about 20,000 daltons (Da), about 500 to about 15,000 Da, about 1000 to about 10,000 Da. In some embodiments of the compositions and methods described herein, a MT activator (e.g., MT1 and/or MT2 isoform) or agonist comprises a small molecule that binds to a MT1 and/or MT2 isoform and mimics metal binding, e.g. zinc binding.

In some embodiments, a MT activator for use in the methods and compositions as disclosed herein can be a peptide, e.g., a metallothionein-derived peptide fragment as disclosed in U.S. Patent Application 2010/0166759, which is incorporated herein in its entirety by reference.

In some embodiments, a MT activator for use in the methods and compositions as disclosed herein is a peptide, or a subsequence of a metallothionein selected from the group consisting of metallothionein-1 A (MT1A), metallothionein-1B (MT1B), metallothionein-1E (MT1E), metallothionein-1F (MT1F), metallothionein-1G (MT1G), metallothionein-1H (MT1H), metallothionein-1I (MT1I), metallothionein-1 K (MT1K), metallothionein-1L (MT1L), metallothionein-1R (MT1R), metallothionein-1X (MT1X), metallothionein-2 (MT2), metallothionein-3 (MT3) and metallothionein-4 (MT4).

In some embodiments, a MT activator for use in the methods and compositions as disclosed herein is a peptide, or a subsequence of a metallothionein selected from the group consisting of:

(SEQ ID NO: 29)
KKSSCSCSPVGSAK
(SEQ ID NO: 30)
AQGSISKGASDKSS
(SEQ ID NO: 31)
MDPNSSSAAGDSST
(SEQ ID NO: 32)
SAGSSKSKESKSTS
(SEQ ID NO: 33)
AQGSICKGASDKSS
(SEQ ID NO: 34)
MDPNCSCAAGDSST
(SEQ ID NO: 35)
SAGSCKCKESKSTS
(SEQ ID NO: 36)
KGGEAAEAEAEK,
(SEQ ID NO: 37)
KKSCCSCCPMSCAK
(SEQ ID NO: 38)
KKCCGSCCPVGCAK
(SEQ ID NO: 39)
KKSCCSCCPVGCAK
(SEQ ID NO: 40)
KKSCCSCCPVGCSK
(SEQ ID NO: 41)
KKSCCSCCPVGCAK
(SEQ ID NO: 42)
KKSCCSCCPLGCAK
(SEQ ID NO: 43)
KKSCCSCCPVGCAK
(SEQ ID NO: 44)
KKSCCSCCPVGCAK
(SEQ ID NO: 45)
KKSCCSCCPVGCAK
(SEQ ID NO: 46)
KKSCCSCCPMGCAK
(SEQ ID NO: 47)
KKSCCSCCPVGCAK
(SEQ ID NO: 48)
KKSCCSCCPVGCAK
(SEQ ID NQ: 49)
KKSCCSCCPAECEK
(SEQ ID NO: 50)
RKSCCPCCPPGCAK
(SEQ ID NO: 51)
AQGCICKGASEKCS
(SEQ ID NO: 52)
AQGCVCKGSSEKCS
(SEQ ID NO: 53)
AQGCVCKGASEKCS
(SEQ ID NO: 54)
AQGCVCKGASEKCS
(SEQ ID NO: 55)
AQGCICKGASEKCS
(SEQ ID NO: 56)
AQGCICKGASEKCS
(SEQ ID NO: 57)
AQGCICKGASEKCS
(SEQ ID NO: 58)
AQGCICKGASEKCS
(SEQ ID NO: 59)
AQGCICKGTSDKCS
(SEQ ID NO: 60)
AQGCVCKGASEKCS
(SEQ ID NO: 61)
AQGCICKGTSDKCS
(SEQ ID NO: 62
AQGCICKGASDKCS
(SEQ ID NO: 63)
AKDCVCKGGEAAEAEAEKCS
(SEQ ID NO: 64)
ARGCICKGGSDKCS
(SEQ ID NO: 65)
MDPNCSCATGGSCT
(SEQ ID NO: 66)
MDPNCSCTTGGSCA
(SEQ ID NO: 67)
MDPNCSCATGGSCT
(SEQ ID NO: 68)
MDPNCSCAAGVSCT
(SEQ ID NO: 69)
MDPNCSCAAGVSCT
(SEQ ID NO: 70)
MDPNCSCEAGGSCA
(SEQ ID NO: 71)
MDPNCSCAAGVSCT
(SEQ ID NO: 72)
MDPNCSCAAAGVSCT
(SEQ ID NO: 73)
MDPNCSCSPVGSCA
(SEQ ID NO: 74)
MDPNCSCATGGSCS
(SEQ ID NO: 75)
MDPNCSCDPVGSCA
(SEQ ID NO: 76)
MDPNCSCAAGDSCT
(SEQ ID NO: 77)
MDPETCPCPSGGSCT
(SEQ ID NO: 78)
MDPRECVCMSGGICM
(SEQ ID NO: 79)
CTGSCKCKECKCNS
(SEQ ID NO: 80)
CAGSCKCKECKCTS
(SEQ ID NO: 81)
CAGSCKCKECKCTS
(SEQ ID NO: 82)
CAGSCKCKECKCTS
(SEQ ID NO: 83)
CASSCKCKECKCTS
(SEQ ID NO: 84)
CAGSCKCKKCKCTS
(SEQ ID NO: 85)
CAGSCKCKECKCTS
(SEQ ID NO: 86)
CASSCKCKECKCTS
(SEQ ID NO: 87)
CAGSCKCKECKCTS
(SEQ ID NO: 88)
CASSCKCKECKCTS
(SEQ ID NO: 89)
CAGSCKCKECKCTS
(SEQ ID NO: 90)
CAGSCKCKECKCTS
(SEQ ID NO: 91)
CADSCKCEGCKCTS
(SEQ ID NO: 92)
CGDNCKCTTCNCKT.

In some embodiments of the compositions and methods described herein, a MT activator (e.g., MT1 and/or MT2 isoform) or agonist is an RNA or DNA aptamer that binds or physically interacts with an isoform of MT1 and/or MT2, and enhances or promotes interactions between MT1 and/or MT2 and its metal binding ligand. In some embodiments of the compositions and methods described herein, the aptamer comprises at least one RNA or DNA aptamer that binds to at least one isoform of MT1 and/or MT2. In some embodiments of the compositions and methods described herein, a MT activator (e.g., MT1 and/or MT2 isoform) or agonist is an RNA or DNA aptamer that binds or physically interacts with at least one isoform of MT1 and/or MT2, and increases, enhances, or promotes downstream MT1 and/or MT2 signaling, e.g., a decreased in IL-10 secretion or production from Tr1 cells.

In some embodiments of the compositions and methods described herein, a MT activator (e.g., MT1 and/or MT2 isoform) or agonist comprises at least one MT1 and/or MT2 structural analog. The term MT1 and/or MT2 isoform structural analog, as used herein, refer to compounds that have a similar three dimensional structure as part of that of an isoform of MT1 and/or MT2 under physiological conditions in vitro or in vivo, wherein the structural analog has at least partially mimics or increases MT1 and/or MT2 biological activity. Suitable MT1 and/or MT2 isoform structural analogs can be designed and synthesized through molecular modeling of MT1 and/or MT2. The MT1 and/or MT2 isoform structural analogs can be monomers, dimers, or higher order multimers in any desired combination of the same or different structures to obtain improved affinities and biological effects.

A MT activator (e.g., MT1 and/or MT2 isoform) or agonists for use in the compositions and methods described herein can be identified or characterized using methods known in the art, such as protein-protein binding assays, biochemical screening assays, immunoassays, and cell-based assays, which are well known in the art.

For example, to identify a molecule that increases interaction between MTs and a metal ligand, e.g., zinc, binding assays can be used. For example, a MT1 and/or MT2 isoform can be immobilized on a microtiter plate by covalent or non-covalent attachment. The assay is performed by adding the non-immobilized component (ligand or receptor polypeptide or metal ligand), which can be labeled by a detectable label, to the immobilized component, in the presence or absence of the testing molecule. When the reaction is complete, the non-reacted components are removed and binding complexes are detected. If formation of binding complexes is enhanced or increased by the presence of the testing molecule, the testing molecule can be a candidate MT activator (e.g., MT1 and/or MT2 isoform) or agonist that increases or promotes binding between Mt1 and/or MT2 and its metal ligand. Cell-based assays can also be used to identify MT1 and/or MT2 activators or agonists. For example, the candidate agent can be added to a cell alone or in the presence of a MT1 and/or MT2 isoform to be screened for a particular activity (e.g., decrease of IL-10 production from Tr1 cells), and the ability of the candidate to increase the activity of interest or to mimic MT1 and/or MT2 activity indicates that the testing molecule is a MT activator (e.g., MT1 and/or MT2 isoform) or agonist. In other embodiments, by detecting and/or measuring levels of MT1 and/or MT2 isoform gene expression, activator or agonist molecules that increase an isoform of MT1 and/or MT2 gene expression can be tested. MT1 and/or MT2 isoform gene expression can be detected and/or measured by a variety of methods, such as real time RT-PCR, enzyme-linked immunosorbent assay (“ELISA”), Northern blotting, or flow cytometry, and as known to one of ordinary skill in the art.

As used herein, in regard to a MT1 and/or MT2 modulator, “selectively binds” or “specifically binds” or “specific for” refers to the ability of a MT1 and/or MT2 inhibitor/antagonist or a MT1 and/or MT2 activator/agonist as described herein, such as, for example, a MT1 and/or MT2 antagonist antibody or a MT1 and/or MT2 antigen-binding fragment thereof, to bind to a target, such as MT1 and/or MT2 isoform, with a K_{D} 10⁻⁵ M (10000 nM) or less, e.g., 10⁻⁶ M or less, 10⁻⁷ M or less, 10⁻⁸ M or less, 10⁻⁹ M or less, 10⁻¹⁰ M or less, 10⁻¹¹ M or less, or 10⁻¹²M or less. For example, if a MT1 and/or MT2 inhibitor/antagonist or a MT1 and/or MT2 activator/agonist described herein binds to a MT1 and/or MT2 isoform with a K_{D} of 10⁻⁵ M or lower, but not to a related MT enzyme, then the agent is said to specifically bind to MT1 and/or MT2. Specific binding can be influenced by, for example, the affinity and avidity of, for example, the MT1 and/or MT2 inhibitor/antagonist or MT1 and/or MT2 activator/agonist antibody or antigen-binding fragment thereof and the concentration of polypeptide agent. The person of ordinary skill in the art can determine appropriate conditions under which the polypeptide agents described herein selectively bind the targets using any suitable methods, such as titration of a polypeptide agent in a suitable cell binding assay.

Antibodies specific for or that selectively bind MT1 and/or MT2, whether a MT1 and/or MT2 activator/agonist antibody or a MT1 and/or MT2 blocking (e.g., neutralizing) or antagonist antibody, suitable for use in the compositions and for practicing the methods described herein are preferably monoclonal, and can include, but are not limited to, human, humanized or chimeric antibodies, comprising single chain antibodies, Fab fragments, F(ab′) fragments, fragments produced by a Fab expression library, and/or binding fragments of any of the above. Antibodies also refer to immunoglobulin molecules and immunologically active portions of immunoglobulin molecules, i.e., molecules that contain antigen or target binding sites or “antigen-binding fragments.” The immunoglobulin molecules described herein can be of any type (e.g., IgG, IgE, IgM, IgD, IgA and IgY), class (e.g., IgG1, IgG2, IgG3, IgG4, IgA1 and IgA2) or subclass of immunoglobulin molecule, as is understood by one of skill in the art.

In some embodiments of the compositions and methods described herein, a MT1 and/or MT2 inhibitor/antagonist or a MT1 and/or MT2 activator/agonist as described herein is a monoclonal MT1 and/or MT2 antibody fragment or antigen-binding fragment.

In some embodiments of the compositions and methods described herein, a MT1 and/or MT2 inhibitor/antagonist or a MT1 and/or MT2 activator/agonist as described herein is a MT1 and/or MT2 antibody fragment or antigen-binding fragment. Examples of antibody fragments encompassed by the terms antibody fragment or antigen-binding fragment include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the CH1 domain; (v) the Fv fragment having the V_{L} and V_{H} domains of a single arm of an antibody; (vi) a dAb fragment (Ward et al., Nature 341, 544-546 (1989)) which consists of a V_{H} domain or a V_{L} domain; (vii) isolated CDR regions; (viii) F(ab′)₂ fragments, a bivalent fragment including two Fab′ fragments linked by a disulphide bridge at the hinge region; (ix) single chain antibody molecules (e.g. single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (V_{H}-C_{H}1-V_{H}-C_{H}1) which, together with complementary light chain polypeptides, form a pair of antigen binding regions (Zapata et al. Protein Eng. 8(10):1057-1062 (1995); and U.S. Pat. No. 5,641,870); and modified versions of any of the foregoing (e.g., modified by the covalent attachment of polyalkylene glycol (e.g., polyethylene glycol, polypropylene glycol, polybutylene glycol) or other suitable polymer).

Thus, the data provided herein identify MTs (e.g., MT1 and/or MT2 isoforms) as a critical inducer of TIM-3-mediated T cell exhaustion/dysfunction during chronic conditions. Accordingly, provided herein are novel compositions and methods to modulate chronic immune conditions by inhibiting MT1 and/or MT2 in CD8+ T cells, and resulting activation of immune responses or inhibition of T cell exhaustion phenotypes.

Accordingly, provided herein are methods for the treatment of chronic immune conditions, such as cancer, persistent infections, and autoimmune disorders in a subject in need thereof. These methods involve, in part, administering to a subject a therapeutically effective amount of a MT1 and/or MT2 modulating agent (i.e., activating or inhibiting) described herein. These methods are particularly aimed at therapeutic treatments of human subjects having a condition in which one or more immune cell populations, such as a CD8+ T cell population, are functionally exhausted, and at therapeutic treatments of human subjects having a condition in which it is desired to cause or induce one or more immune cell populations, such as a CD8+ T cell population, to become functionally exhausted.

Accordingly, provided herein, in some aspects are methods for the treatment of a chronic immune conditions, e.g., chronic infection and/or cancer in a subject in need thereof, comprising administering to a subject an effective amount of a composition comprising an MT1 and/or MT2 inhibitor or antagonist that decreases CD8+ T cell exhaustion.

In some embodiments of these methods and all such methods described herein, a MT1 and/or MT2 inhibitor inhibits MT1 and/or MT2 mediated signal transduction.

In some embodiments of these methods and all such methods described herein, the method further comprises administering any of the MT1 and/or MT2 inhibitors or antagonists described herein.

In regard to the methods of treating chronic immune conditions, by decreasing CD8+ T-cell exhaustion by inhibiting MT1 and/or MT2 activity, immunosuppression of a host immune response plays a role in a variety of chronic immune conditions, such as in persistent infection and tumor immunosuppression. Recent evidence indicates that this immunosuppression can be mediated by immune inhibitory receptors expressed on the surface of an immune cell, and their interactions with their ligands. For example, CD8+ T-cells can enter a state of “functional exhaustion,” or “unresponsiveness” whereby they express inhibitory receptors that prevent antigen-specific responses, such as proliferation and cytokine production. Accordingly, by decreasing the activity and/or expression of MTs, using a MT1 and/or MT2 inhibitors as described herein, an immune response to a persistent infection or to a cancer or tumor that is suppressed, inhibited, or unresponsive, can be enhanced or uninhibited.

As used herein, an “immune response” refers to a response by a cell of the immune system, such as a B cell, T cell (CD4 or CD8), regulatory T cell, antigen-presenting cell, dendritic cell, monocyte, macrophage, NKT cell, NK cell, basophil, eosinophil, or neutrophil, to a stimulus. In some embodiments, the response is specific for a particular antigen (an “antigen-specific response”), and refers to a response by a CD4 T cell, CD8 T cell, or B cell via an antigen-specific receptor. In some embodiments, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response.

As used herein, “unresponsiveness” or “functional exhaustion” with regard to immune cells includes refractivity of immune cells to stimulation, such as stimulation via an activating receptor or a cytokine. Unresponsiveness can occur, for example, because of exposure to immunosuppressants, exposure to high or constant doses of antigen, or through the activity of inhibitor receptors, such as TIM-3. As used herein, the term “unresponsiveness” includes refractivity to activating receptor-mediated stimulation. Such refractivity is generally antigen-specific and persists after exposure to the antigen has ceased. Unresponsive immune cells can have a reduction of at least 10%, 20%, 30%, 40%, 50%, 60%, 70%, 80%, 90%, 95%, or even 100% in cytotoxic activity, cytokine production, proliferation, trafficking, phagocytotic activity, or any combination thereof, relative to a corresponding control immune cell of the same type.

Accordingly, in some embodiments of the methods of treating chronic immune conditions by decreasing CD8+ T-cell exhaustion and inhibiting MT1 and/or MT2 activity described herein, the subject being administered a MT1 and/or MT2 inhibitor has or has been diagnosed as having a cancer or tumor.

Studies have shown defective or suppressed immune responses in patients diagnosed with cancer. Described herein is the novel finding that the decrease of MTs, e.g., MT1 and/or MT2 expression decreases or inhibits functional exhaustion of CD8+ T cells, and inhibits tumor or cancer growth. Furthermore, described herein is the novel finding that targeting MT1 and/or MT2 signaling, using, for example, a MT1 and/or MT2 inhibiting agent as described herein, restores or promotes the responsiveness of these CD8+ T cells, such that a cancer or tumor is inhibited or reduced.

A “cancer” or “tumor” as used herein refers to an uncontrolled growth of cells which interferes with the normal functioning of the bodily organs and systems. A subject that has a cancer or a tumor is a subject having objectively measurable cancer cells present in the subject's body. Included in this definition are benign and malignant cancers, as well as dormant tumors or micrometastases. Cancers which migrate from their original location and seed vital organs can eventually lead to the death of the subject through the functional deterioration of the affected organs. Hemopoietic cancers, such as leukemia, are able to out-compete the normal hemopoietic compartments in a subject, thereby leading to hemopoietic failure (in the form of anemia, thrombocytopenia and neutropenia) ultimately causing death.

By “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

Examples of cancer include but are not limited to, carcinoma, lymphoma, blastoma, sarcoma, and leukemia. More particular examples of such cancers include, but are not limited to, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

In some embodiments of these methods and all such methods described herein, the methods further comprise administering a tumor or cancer antigen to a subject being administered a MT1 and/or MT2 activating agent as described herein.

A number of tumor antigens have been identified that are associated with specific cancers. As used herein, the terms “tumor antigen” and “cancer antigen” are used interchangeably to refer to antigens which are differentially expressed by cancer cells and can thereby be exploited in order to target cancer cells. Cancer antigens are antigens which can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), and fusion proteins resulting from internal deletions or chromosomal translocations. Still other cancer antigens can be encoded by viral genes such as those carried on RNA and DNA tumor viruses. Many tumor antigens have been defined in terms of multiple solid tumors: MAGE 1, 2, & 3, defined by immunity; MART-1/Melan-A, gp100, carcinoembryonic antigen (CEA), HER-2, mucins (i.e., MUC-1), prostate-specific antigen (PSA), and prostatic acid phosphatase (PAP). In addition, viral proteins such as hepatitis B (HBV), Epstein-Ban (EBV), and human papilloma (HPV) have been shown to be important in the development of hepatocellular carcinoma, lymphoma, and cervical cancer, respectively. However, due to the immunosuppression of patients diagnosed with cancer, the immune systems of these patients often fail to respond to the tumor antigens.

In some embodiments of these methods and all such methods described herein, the methods further comprise administering an anti-cancer therapy or agent to a subject in addition to a MT1 and/or MT2 inhibiting agent as described herein.

The term “anti-cancer therapy” refers to a therapy useful in treating cancer. Examples of anti-cancer therapeutic agents include, but are not limited to, e.g., surgery, chemotherapeutic agents, growth inhibitory agents, cytotoxic agents, agents used in radiation therapy, anti-angiogenesis agents, apoptotic agents, anti-tubulin agents, and other agents to treat cancer, such as anti-HER-2 antibodies (e.g., HERCEPTIN®), anti-CD20 antibodies, an epidermal growth factor receptor (EGFR) antagonist (e.g., a tyrosine kinase inhibitor), HER1/EGFR inhibitor (e.g., erlotinib (TARCEVA®)), platelet derived growth factor inhibitors (e.g., GLEEVEC™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also specifically contemplated for the methods described herein.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small molecule toxins or enzymatically active toxins of bacterial, fungal, plant or animal origin, including active fragments and/or variants thereof.

In some embodiments of these methods and all such methods described herein, the methods further comprise administering a chemotherapeutic agent to the subject being administered a MT1 and/or MT2 inhibitor agents or combination thereof as described herein in methods and compositions to increase the activity of exhausted CD8+ cells.

Non-limiting examples of chemotherapeutic agents can include include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE, vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (TYKERB.); inhibitors of PKC-alpha, Raf, H-Ras, EGFR (e.g., erlotinib (TARCEVA®)) and VEGF-A that reduce cell proliferation and pharmaceutically acceptable salts, acids or derivatives of any of the above. In addition, the methods of treatment can further include the use of radiation or radiation therapy.

As used herein, the terms “chemotherapy” or “chemotherapeutic agent” refer to any chemical agent with therapeutic usefulness in the treatment of diseases characterized by abnormal cell growth. Such diseases include tumors, neoplasms and cancer as well as diseases characterized by hyperplastic growth. Chemotherapeutic agents as used herein encompass both chemical and biological agents. These agents function to inhibit a cellular activity upon which the cancer cell depends for continued survival. Categories of chemotherapeutic agents include alkylating/alkaloid agents, antimetabolites, hormones or hormone analogs, and miscellaneous antineoplastic drugs. Most if not all of these agents are directly toxic to cancer cells and do not require immune stimulation. In one embodiment, a chemotherapeutic agent is an agent of use in treating neoplasms such as solid tumors. In one embodiment, a chemotherapeutic agent is a radioactive molecule. One of skill in the art can readily identify a chemotherapeutic agent of use (e.g. see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2.sup.nd ed., .COPYRGT. 2000 Churchill Livingstone, Inc; Baltzer L, Berkery R (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993).

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one time administration and typical dosages range from 10 to 200 units (Grays) per day.

By “reduce” or “inhibit” in terms of the cancer treatment methods described herein is meant the ability to cause an overall decrease preferably of 20% or greater, 30% or greater, 40% or greater, 45% or greater, more preferably of 50% or greater, of 55% or greater, of 60% or greater, of 65% or greater, of 70% or greater, and most preferably of 75% or greater, 80% or greater, 85% or greater, 90% or greater, or 95% or greater, for a given parameter or symptom. Reduce or inhibit can refer to, for example, the symptoms of the disorder being treated, the presence or size of metastases or micrometastases, the size of the primary tumor, the presence or the size of the dormant tumor, or the load of infectious agent.

In other embodiments of the methods of treating chronic immune conditions by decreasing CD8+ T-cell exhaustion and inhibiting MT1 and/or MT2 activity as described herein, the subject being administered a MT1 and/or MT2 inhibitor has or has been diagnosed as having a persistent infection with a bacterium, virus, fungus, or parasite.

“Persistent infections” refer to those infections that, in contrast to acute infections, are not effectively cleared by the induction of a host immune response. During such persistent infections, the infectious agent and the immune response reach equilibrium such that the infected subject remains infectious over a long period of time without necessarily expressing symptoms. Persistent infections often involve stages of both silent and productive infection without rapidly killing or even producing excessive damage of the host cells. Persistent infections include for example, latent, chronic and slow infections. Persistent infection occurs with viruses including, but not limited to, human T-Cell leukemia viruses, Epstein-Barr virus, cytomegalovirus, herpes viruses, varicella-zoster virus, measles, papovaviruses, prions, hepatitis viruses, adenoviruses, parvoviruses and papillomaviruses.

In a “chronic infection,” the infectious agent can be detected in the subject at all times. However, the signs and symptoms of the disease can be present or absent for an extended period of time. Non-limiting examples of chronic infection include hepatitis B (caused by hepatitis B virus (HBV)) and hepatitis C (caused by hepatitis C virus (HCV)) adenovirus, cytomegalovirus, Epstein-Barr virus, herpes simplex virus 1, herpes simplex virus 2, human herpes virus 6, varicella-zoster virus, hepatitis B virus, hepatitis D virus, papilloma virus, parvovirus B19, polyomavirus BK, polyomavirus JC, measles virus, rubella virus, human immunodeficiency virus (HIV), human T cell leukemia virus I, and human T cell leukemia virus II. Parasitic persistent infections can arise as a result of infection by, for example, Leishmania, Toxoplasma, Trypanosoma, Plasmodium, Schistosoma, and Encephalitozoon.

In a “latent infection,” the infectious agent (such as a virus) is seemingly inactive and dormant such that the subject does not always exhibit signs or symptoms. In a latent viral infection, the virus remains in equilibrium with the host for long periods of time before symptoms again appear; however, the actual viruses cannot typically be detected until reactivation of the disease occurs. Non-limiting examples of latent infections include infections caused by herpes simplex virus (HSV)-1 (fever blisters), HSV-2 (genital herpes), and varicella zoster virus VZV (chickenpox-shingles).

In a “slow infection,” the infectious agents gradually increase in number over a very long period of time during which no significant signs or symptoms are observed. Non-limiting examples of slow infections include AIDS (caused by HIV-1 and HIV-2), lentiviruses that cause tumors in animals, and prions.

In addition, persistent infections that can be treated using the methods described herein include those infections that often arise as late complications of acute infections. For example, subacute sclerosing panencephalitis (SSPE) can occur following an acute measles infection or regressive encephalitis can occur as a result of a rubella infection.

The mechanisms by which persistent infections are maintained can involve modulation of virus and cellular gene expression and modification of the host immune response. Reactivation of a latent infection can be triggered by various stimuli, including changes in cell physiology, superinfection by another virus, and physical stress or trauma. Host immunosuppression is often associated with reactivation of a number of persistent virus infections.

Additional examples of infectious viruses include: Retroviridae; Picornaviridae (for example, polio viruses, hepatitis A virus; enteroviruses, human coxsackie viruses, rhinoviruses, echoviruses); Calciviridae (such as strains that cause gastroenteritis); Togaviridae (for example, equine encephalitis viruses, rubella viruses); Flaviridae (for example, dengue viruses, encephalitis viruses, yellow fever viruses); Coronaviridae (for example, coronaviruses); Rhabdoviridae (for example, vesicular stomatitis viruses, rabies viruses); Filoviridae (for example, ebola viruses); Paramyxoviridae (for example, parainfluenza viruses, mumps virus, measles virus, respiratory syncytial virus); Orthomyxoviridae (for example, influenza viruses); Bungaviridae (for example, Hantaan viruses, bunga viruses, phleboviruses and Nairo viruses); Arena viridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and HSV-2, varicella zoster virus, cytomegalovirus (CMV), herpes viruses); Poxviridae (variola viruses, vaccinia viruses, pox viruses); and Iridoviridae (such as African swine fever virus); and unclassified viruses (for example, the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses). The compositions and methods described herein are contemplated for use in treating infections with these viral agents.

Examples of fungal infections include but are not limited to: aspergillosis; thrush (caused by Candida albicans); cryptococcosis (caused by Cryptococcus); and histoplasmosis. Thus, examples of infectious fungi include, but are not limited to, Cryptococcus neoformans, Histoplasma capsulatum, Coccidioides immitis, Blastomyces dermatitidis, Chlamydia trachomatis, Candida albicans. The compositions and methods described herein are contemplated for use in treating infections with these fungal agents.

Examples of infectious bacteria include: Helicobacter pylori, Borelia burgdorferi, Legionella pneumophilia, Mycobacteria sps (such as M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracia, Corynebacterium diphtheriae, corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, and Actinomyces israelli. The compositions and methods described herein are contemplated for use in treating infections with these bacterial agents. Other infectious organisms (such as protists) include: Plasmodium falciparum and Toxoplasma gondii. The compositions and methods described herein are contemplated for use in treating infections with these agents.

In some embodiments of the aspects described herein, the methods further comprise administering an effective amount of a viral, bacterial, fungal, or parasitic antigen in conjunction with the MT1 and/or MT2 inhibitor. Non-limiting examples of suitable viral antigens include: influenza HA, NA, M, NP and NS antigens; HIV p24, pol, gp41 and gp120; Metapneumovirus (hMNV) F and G proteins; Hepatitis C virus (HCV) E1, E2 and core proteins; Dengue virus (DEN1-4) E1, E2 and core proteins; Human Papilloma Virus L1 protein; Epstein Barr Virus gp220/350 and EBNA-3A peptide; Cytomegalovirus (CMV) gB glycoprotein, gH glycoprotein, pp65, IE1 (exon 4) and pp 150; Varicella Zoster virus (VZV) 1E62 peptide and glycoprotein E epitopes; Herpes Simplex Virus Glycoprotein D epitopes, among many others. The antigenic polypeptides can correspond to polypeptides of naturally occurring animal or human viral isolates, or can be engineered to incorporate one or more amino acid substitutions as compared to a natural (pathogenic or non-pathogenic) isolate.

In some embodiments, the methods described herein comprise administering an effective amount of a MT1 and/or MT2 modulator (i.e., inhibitor or activator) described herein to a subject in order to alleviate a symptom of persistent infection. As used herein, “alleviating a symptom of a persistent infection” is ameliorating any condition or symptom associated with the persistent infection. Alternatively, alleviating a symptom of a persistent infection can involve reducing the infectious microbial (such as viral, bacterial, fungal or parasitic) load in the subject relative to such load in an untreated control. As compared with an equivalent untreated control, such reduction or degree of prevention is at least 5%, 10%, 20%, 40%, 50%, 60%, 80%, 90%, 95%, or more as measured by any standard technique. Desirably, the persistent infection is cleared, or pathogen replication has been suppressed, as detected by any standard method known in the art, in which case the persistent infection is considered to have been treated. A patient who is being treated for a persistent infection is one who a medical practitioner has diagnosed as having such a condition. Diagnosis can be by any suitable means. Diagnosis and monitoring can involve, for example, detecting the level of microbial load in a biological sample (for example, a tissue biopsy, blood test, or urine test), detecting the level of a surrogate marker of the microbial infection in a biological sample, detecting symptoms associated with persistent infections, or detecting immune cells involved in the immune response typical of persistent infections (for example, detection of antigen specific T cells that are anergic and/or functionally impaired).

In other aspects, provided herein are methods for the treatment of a chronic immune condition in a subject in need thereof, comprising administering to a subject in need thereof an effective amount of a composition comprising a MT1 and/or MT2 activator or agonist that increases T cell exhaustion.

### MT Inhibitors to Treat Autoimmune Diseases

In some embodiments of the methods of treating an autoimmune disease or disorder as described herein, the subject being administered a MT1 and/or MT2 inhibitor has or has been diagnosed with an autoimmune disease or disorder.

As used herein, an “autoimmune disease” refers to a class of diseases in which a subject's own antibodies react with host tissue or in which immune effector T cells are autoreactive to endogenous self-peptides and cause destruction of tissue. Thus an immune response is mounted against a subject's own antigens, referred to as self-antigens. A “self-antigen” as used herein refers to an antigen of a normal host tissue. Normal host tissue does not include cancer cells.

Accordingly, in some embodiments of these methods and all such methods described herein, the autoimmune diseases to be treated or prevented using the methods described herein, include, but are not limited to: rheumatoid arthritis, Crohn's disease or colitis, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjogren's syndrome, insulin resistance, and autoimmune diabetes mellitus (type 1 diabetes mellitus; insulin-dependent diabetes mellitus). Autoimmune disease has been recognized also to encompass atherosclerosis and Alzheimer's disease. In some embodiments of the aspects described herein, the autoimmune disease is selected from the group consisting of multiple sclerosis, type-I diabetes, Hashimoto's thyroiditis, Crohn's disease or colitis, rheumatoid arthritis, systemic lupus erythematosus, gastritis, autoimmune hepatitis, hemolytic anemia, autoimmune hemophilia, autoimmune lymphoproliferative syndrome (ALPS), autoimmune uveoretinitis, glomerulonephritis, Guillain-Barre syndrome, psoriasis and myasthenia gravis.

In some embodiments, a subject being administered a MT1 and/or MT inhibitor as disclosed herein has or has been diagnosed with host versus graft disease (HVGD). In a further such embodiment, the subject being treated with the methods described herein is an organ or tissue transplant recipient. In other embodiments of the methods are used for increasing transplantation tolerance in a subject. In some such embodiments, the subject is a recipient of an allogenic transplant. The transplant can be any organ or tissue transplant, including but not limited to heart, kidney, liver, skin, pancreas, bone marrow, skin or cartilage. “Transplantation tolerance,” as used herein, refers to a lack of rejection of the donor organ by the recipient's immune system.

The terms “subject” and “individual” as used in regard to any of the methods described herein are used interchangeably herein, and refer to an animal, for example a human, recipient of the bispecific or multispecific polypeptide agents described herein. For treatment of disease states which are specific for a specific animal such as a human subject, the term “subject” refers to that specific animal. The terms “non-human animals” and “non-human mammals” are used interchangeably herein, and include mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like. Production mammal, e.g. cow, sheep, pig, and the like are also encompassed in the term subject.

As used herein, in regard to any of the compositions and methods comprising a MT1 and/or MT2 modulator (i.e., inhibitors or activators) or combinations thereof described herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder associated with a chronic immune condition, such as, but not limited to, a chronic infection or a cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

The term “effective amount” as used herein refers to the amount of a MT1 and/or MT2 modulator (i.e., inhibitor or activator), or combinations thereof described herein, needed to alleviate at least one or more symptom of the disease or disorder being treated, and relates to a sufficient amount of pharmacological composition to provide the desired effect, i.e., reverse the functional exhaustion of antigen-specific T cells in a subject having a chronic immune condition, such as cancer or hepatitis C. The term “therapeutically effective amount” therefore refers to an amount of a MT1 and/or MT2 modulator (i.e., inhibitor or activator), or combinations thereof described herein, using the methods as disclosed herein, that is sufficient to provide a particular effect when administered to a typical subject. An effective amount as used herein would also include an amount sufficient to delay the development of a symptom of the disease, alter the course of a symptom disease (for example but not limited to, slow the progression of a symptom of the disease), or reverse a symptom of the disease. Thus, it is not possible to specify the exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the 1050 (i.e., the concentration of a MT1 and/or MT2 modulator (i.e., inhibitor or activator)), or combinations thereof described herein, which achieves a half-maximal inhibition of measured function or activity) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

### Modes of Administration

A MT1 and/or MT2 modulator (i.e., inhibitors and activators), or combinations thereof described herein, described herein can be administered to a subject in need thereof by any appropriate route which results in an effective treatment in the subject. As used herein, the terms “administering,” and “introducing” are used interchangeably and refer to the placement of a MT1 and/or MT2 modulator (i.e., inhibitor or activator), or a combination thereof, into a subject by a method or route which results in at least partial localization of such agents at a desired site, such as a site of inflammation, such that a desired effect(s) is produced.

In some embodiments, a MT1 and/or MT2 modulator (i.e., inhibitor or activator) or combination thereof is administered to a subject having a chronic immune condition by any mode of administration that delivers the agent systemically or to a desired surface or target, and can include, but is not limited to, injection, infusion, instillation, and inhalation administration. To the extent that polypeptide agents can be protected from inactivation in the gut, oral administration forms are also contemplated. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, a MT1 and/or MT2 modulators (i e., inhibitors or activators) for use in the methods described herein are administered by intravenous infusion or injection. In some embodiments, a MT1 and/or MT2 modulator (i.e., inhibitor or activator) or combination thereof is not administered systemically, but rather, depending on the indication, a specific subpopulation of T-cells is treated with the MT1 and/or MT2 modulator ex vivo, and the treated cells are then administered via any suitable means, e.g., injection, into the subject. For example, for the treatment of a subject with an autoimmune disease or disorder, Tr1 cells obtained from the subject are treated with a MT1 and/or MT2 inhibitor ex vivo, and the treated Tr1 cells are subsequently administered to the subject via suitable administration route. Similarly, for the treatment of a subject to decrease the production of IL-10 from Tr1 cells, Tr1 cells obtained from the subject are treated with a MT1 and/or MT2 activator ex vivo, and the treated Tr1 cells are subsequently administered to the subject via suitable administration route. In alternative embodiments, for the treatment of a subject with a cancer and/or a chronic immune disease such as a chronic infection, a population of CD8+ T-cells (including functionally exhausted CD8+ T-cells) obtained from the subject are treated with a MT1 and/or MT2 inhibitor ex vivo, and the treated CD8+ cells are subsequently administered to the subject via suitable administration route.

The phrases “parenteral administration” and “administered parenterally” as used herein, refer to modes of administration other than enteral and topical administration, usually by injection. The phrases “systemic administration,” “administered systemically”, “peripheral administration” and “administered peripherally” as used herein refer to the administration of a MT1 and/or MT2 modulator (i.e., inhibitor or activator), or combination thereof, other than directly into a target site, tissue, or organ, such as a tumor site, such that it enters the subject's circulatory system and, thus, is subject to metabolism and other like processes.

For the clinical use of the methods described herein, administration of a MT1 and/or MT2 modulators (i.e., inhibitors or activators), or combinations thereof described herein, can include formulation into pharmaceutical compositions or pharmaceutical formulations for parenteral administration, e.g., intravenous; mucosal, e.g., intranasal; ocular, or other mode of administration. In some embodiments, a MT1 and/or MT2 modulators (i e., inhibitors or activators), or combinations thereof described herein, can be administered along with any pharmaceutically acceptable carrier compound, material, or composition which results in an effective treatment in the subject. Thus, a pharmaceutical formulation for use in the methods described herein can contain a MT1 and/or MT2 modulator (i.e., inhibitor or activator), or combination thereof, as described herein in combination with one or more pharmaceutically acceptable ingredients.

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the stability, solubility, or activity of, a MT1 and/or MT2 modulator (i.e., inhibitor or activator), or combination thereof. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) excipients, such as cocoa butter and suppository waxes; (8) oils, such as peanut oil, cottonseed oil, safflower oil, sesame oil, olive oil, corn oil and soybean oil; (9) glycols, such as propylene glycol; (10) polyols, such as glycerin, sorbitol, mannitol and polyethylene glycol (PEG); (11) esters, such as ethyl oleate and ethyl laurate; (12) agar; (13) buffering agents, such as magnesium hydroxide and aluminum hydroxide; (14) alginic acid; (15) pyrogen-free water; (16) isotonic saline; (17) Ringer's solution; (19) pH buffered solutions; (20) polyesters, polycarbonates and/or polyanhydrides; (21) bulking agents, such as polypeptides and amino acids (22) serum components, such as serum albumin, HDL and LDL; (23) C2-C12 alcohols, such as ethanol; and (24) other non-toxic compatible substances employed in pharmaceutical formulations. Release agents, coating agents, preservatives, and antioxidants can also be present in the formulation. The terms such as “excipient”, “carrier”, “pharmaceutically acceptable carrier” or the like are used interchangeably herein.

A MT1 and/or MT2 modulator (i.e., inhibitors or activators) or combinations thereof described herein can be specially formulated for administration of the compound to a subject in solid, liquid or gel form, including those adapted for the following: (1) parenteral administration, for example, by subcutaneous, intramuscular, intravenous or epidural injection as, for example, a sterile solution or suspension, or sustained-release formulation; (2) topical application, for example, as a cream, ointment, or a controlled-release patch or spray applied to the skin; (3) intravaginally or intrarectally, for example, as a pessary, cream or foam; (4) ocularly; (5) transdermally; (6) transmucosally; or (79) nasally. Additionally, a bispecific or multispecific polypeptide agent can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

Further embodiments of the formulations and modes of administration of the compositions comprising a MT1 and/or MT2 modulator (i e., inhibitors or activators), or combinations thereof described herein, that can be used in the methods described herein are described below.

Parenteral Dosage Forms.

Parenteral dosage forms of a MT1 and/or MT2 modulator (i.e., inhibitors or activators), or combinations thereof, can also be administered to a subject with a chronic immune condition by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Aerosol Formulations.

A MT1 and/or MT2 modulator (i.e., inhibitor or activator) or combination thereof can be packaged in a pressurized aerosol container together with suitable propellants, for example, hydrocarbon propellants like propane, butane, or isobutane with conventional adjuvants. A MT1 and/or MT2 modulator (i e., inhibitor or activator), or combinations thereof described herein, can also be administered in a non-pressurized form such as in a nebulizer or atomizer. A MT1 and/or MT2 modulator (i e., inhibitor or activator), or combinations thereof described herein, can also be administered directly to the airways in the form of a dry powder, for example, by use of an inhaler.

Suitable powder compositions include, by way of illustration, powdered preparations of a MT1 and/or MT2 modulator (i.e., inhibitor or activator), or combinations thereof described herein, thoroughly intermixed with lactose, or other inert powders acceptable for intrabronchial administration. The powder compositions can be administered via an aerosol dispenser or encased in a breakable capsule which can be inserted by the subject into a device that punctures the capsule and blows the powder out in a steady stream suitable for inhalation. The compositions can include propellants, surfactants, and co-solvents and can be filled into conventional aerosol containers that are closed by a suitable metering valve.

Aerosols for the delivery to the respiratory tract are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic and diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S. and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), contents of all of which are herein incorporated by reference in their entirety.

The formulations of a MT1 and/or MT2 modulator (i.e., inhibitors or activators), or combinations thereof described herein, further encompass anhydrous pharmaceutical compositions and dosage forms comprising the disclosed compounds as active ingredients, since water can facilitate the degradation of some compounds. For example, the addition of water (e.g., 5%) is widely accepted in the pharmaceutical arts as a means of simulating long-term storage in order to determine characteristics such as shelf life or the stability of formulations over time. See, e.g., Jens T. Carstensen, Drug Stability: Principles & Practice, 379-80 (2nd ed., Marcel Dekker, NY, N.Y.: 1995). Anhydrous pharmaceutical compositions and dosage forms of the disclosure can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected. Anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials) with or without desiccants, blister packs, and strip packs.

Controlled and Delayed Release Dosage Forms.

In some embodiments of the aspects described herein, a MT1 and/or MT2 modulator (i e., inhibitor or activator), or combinations thereof described herein, can be administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control a compound of formula (I)'s onset of action, duration of action, plasma levels within the therapeutic window, and peak blood levels. In particular, controlled- or extended-release dosage forms or formulations can be used to ensure that the maximum effectiveness of a compound of formula (I) is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with the MT modulators (i e., inhibitors or activators), or combinations thereof described herein. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, DUOLITE® A568 and DUOLITE® AP143 (Rohm&Haas, Spring House, Pa. USA).

In some embodiments of the methods described herein, a MT modulator (i.e., inhibitor or activator), or combinations thereof described herein, for use in the methods described herein is administered to a subject by sustained release or in pulses. Pulse therapy is not a form of discontinuous administration of the same amount of a composition over time, but comprises administration of the same dose of the composition at a reduced frequency or administration of reduced doses. Sustained release or pulse administrations are particularly preferred when the disorder occurs continuously in the subject, for example where the subject has continuous or chronic symptoms of a viral infection. Each pulse dose can be reduced and the total amount of a MT1 and/or MT2 modulator (i.e., inhibitor or activator), or combinations thereof described herein, administered over the course of treatment to the subject or patient is minimized.

The interval between pulses, when necessary, can be determined by one of ordinary skill in the art. Often, the interval between pulses can be calculated by administering another dose of the composition when the composition or the active component of the composition is no longer detectable in the subject prior to delivery of the next pulse. Intervals can also be calculated from the in vivo half-life of the composition. Intervals can be calculated as greater than the in vivo half-life, or 2, 3, 4, 5 and even 10 times greater the composition half-life. Various methods and apparatus for pulsing compositions by infusion or other forms of delivery to the patient are disclosed in U.S. Pat. Nos. 4,747,825; 4,723,958; 4,948,592; 4,965,251 and 5,403,590.

### FURTHER DEFINITIONS

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology, and molecular biology can be found in The Merck Manual of Diagnosis and Therapy, 18th Edition, published by Merck Research Laboratories, 2006 (ISBN 0-911910-18-2); Robert S. Porter et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006. Definitions of common terms in molecular biology are found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); or Methods in Enzymology: Guide to Molecular Cloning Techniques Vol. 152, S. L. Berger and A. R. Kimmerl Eds., Academic Press Inc., San Diego, USA (1987); Current Protocols in Molecular Biology (CPMB) (Fred M. Ausubel, et al. ed., John Wiley and Sons, Inc.), Current Protocols in Protein Science (CPPS) (John E. Coligan, et. al., ed., John Wiley and Sons, Inc.) and Current Protocols in Immunology (CPI) (John E. Coligan, et. al., ed. John Wiley and Sons, Inc.), which are all incorporated by reference herein in their entireties.

As described herein, an “antigen” is a molecule that is bound by a binding site on a polypeptide agent, such as an antibody. Typically, antigens are bound by antibody ligands and are capable of raising an antibody response in vivo. An antigen can be a polypeptide, protein, nucleic acid or other molecule. In the case of conventional antibodies and fragments thereof, the antibody binding site as defined by the variable loops (L1, L2, L3 and H1, H2, H3) is capable of binding to the antigen. The term “antigenic determinant” refers to an epitope on the antigen recognized by an antigen-binding molecule (such as bispecific polypeptide agent described herein), and more particularly, by the antigen-binding site of said molecule.

As used herein, an “epitope” can be formed both from contiguous amino acids, or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An “epitope” includes the unit of structure conventionally bound by an immunoglobulin V_{H}/V_{L} pair. Epitopes define the minimum binding site for an antibody, and thus represent the target of specificity of an antibody. In the case of a single domain antibody, an epitope represents the unit of structure bound by a variable domain in isolation. The terms “antigenic determinant” and “epitope” can also be used interchangeably herein.

With respect to a target or antigen, the term “ligand interaction site” on the target or antigen means a site, epitope, antigenic determinant, part, domain or stretch of amino acid residues on the target or antigen that is a site for binding to a ligand, receptor or other binding partner, a catalytic site, a cleavage site, a site for allosteric interaction, a site involved in multimerisation (such as homomerization or heterodimerization) of the target or antigen; or any other site, epitope, antigenic determinant, part, domain or stretch of amino acid residues on the target or antigen that is involved in a biological action or mechanism of the target or antigen, e.g., MT1 and/or MT2. More generally, a “ligand interaction site” can be any site, epitope, antigenic determinant, part, domain or stretch of amino acid residues on a target or antigen to which a binding site of a bispecific or multispecific polypeptide agent described herein can bind such that the target or antigen (and/or any pathway, interaction, signalling, biological mechanism or biological effect in which the target or antigen is involved) is modulated.

In the context of an antibody or antigen-binding fragment thereof, the term “specificity” or “specific for” refers to the number of different types of antigens or antigenic determinants to which a particular antibody or antigen-binding fragment thereof can bind. The specificity of an antibody or antigen-binding fragment or portion thereof can be determined based on affinity and/or avidity. The affinity, represented by the equilibrium constant for the dissociation (K_{D}) of an antigen with an antigen-binding protein, is a measure for the binding strength between an antigenic determinant and an antigen-binding site on the antigen-binding protein: the lesser the value of the K_{D}, the stronger the binding strength between an antigenic determinant and the antigen-binding molecule. Alternatively, the affinity can also be expressed as the affinity constant (K_{A}), which is 1/K_{D}). As will be clear to the skilled person, affinity can be determined in a manner known per se, depending on the specific antigen of interest. Accordingly, an antibody or antigen-binding fragment thereof as defined herein is said to be “specific for” a first target or antigen compared to a second target or antigen when it binds to the first antigen with an affinity (as described above, and suitably expressed, for example as a K_{D} value) that is at least 10 times, such as at least 100 times, and preferably at least 1000 times, and up to 10,000 times or more better than the affinity with which said amino acid sequence or polypeptide binds to another target or polypeptide. Preferably, when an antibody or antigen-binding fragment thereof is “specific for” a target or antigen, e.g., MT1 and/or MT2, compared to another target or antigen, it is directed against said target or antigen, but not directed against such other target or antigen.

Avidity is the measure of the strength of binding between an antigen-binding molecule and the pertinent antigen. Avidity is related to both the affinity between an antigenic determinant and its antigen binding site on the antigen-binding molecule, and the number of pertinent binding sites present on the antigen-binding molecule. Typically, antigen-binding proteins will bind to their cognate or specific antigen with a dissociation constant (K_{D} of 10⁻⁵ to 10⁻¹²moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles). Any K_{D} value greater than 10⁻⁴ mol/liter (or any K_{A} value lower than 10⁴ M⁻¹) is generally considered to indicate non-specific binding. The K_{D} for biological interactions which are considered meaningful (e.g., specific) are typically in the range of 10⁻¹⁰ M (0.1 nM) to 10⁻⁵ M (10000 nM). The stronger an interaction is, the lower is its K_{D}. Preferably, a binding site on an MT1 and/or MT2 antagonist antibody or antigen-binding fragment thereof described herein will bind to the desired antigen with an affinity less than 500 nM, preferably less than 200 nM, more preferably less than 10 nM, such as less than 500 pM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as other techniques as mentioned herein.

The term “monoclonal antibody” as used herein in regard to any of the MT1 and/or MT2 inhibiting antibodies described herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each antibody in a monoclonal preparation is directed against the same, single determinant on the antigen. It is to be understood that the term “monoclonal antibody” refers to an antibody that is derived from a single clone, including any eukaryotic, prokaryotic, or phage clone, and not the method by which it is produced. The term “monoclonal antibody” as used herein is not limited to antibodies produced through hybridoma technology, and the modifier “monoclonal” is not to be construed as requiring production of the antibody by any particular method. For example, the monoclonal antibodies to be used in accordance with the invention can be made by the hybridoma method first described by Kohler et al., Nature 256:495 (1975), or later adaptations thereof, or can be made by recombinant DNA methods (see, e.g., U.S. Pat. No. 4,816,567). The “monoclonal antibodies” can also be isolated from phage antibody libraries using the techniques described in Clackson et al., Nature 352:624-628 (1991) or Marks et al., J. Mol. Biol. 222:581-597 (1991), for example.

As used herein in regard to any of the MT1 and/or MT2 inhibiting antibodies described herein, the term “chimeric antibody” refers to an antibody molecule in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)). Chimeric antibody molecules can include, for example, one or more antigen binding domains from an antibody of a mouse, rat, or other species, with human constant regions. A variety of approaches for making chimeric antibodies have been described and can be used to make chimeric antibodies containing the immunoglobulin variable region which recognizes the desired antigen, e.g., MT1 and/or MT2. See, for example, Takeda et al., 1985, Nature 314:452; Cabilly et al., U.S. Pat. No. 4,816,567; Boss et al., Tanaguchi et al., European Patent Publication EP171496; European Patent Publication 0173494, United Kingdom patent GB 2177096B).

Humanized forms of non-human (e.g., murine) antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies can comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

In some embodiments of the compositions and methods comprising any of the MT1 and/or MT2 inhibiting or neutralizing antibodies or antigen-binding fragments thereof described herein, the MT1 and/or MT2 inhibiting antibody or antigen-binding fragment is an antibody derivative. For example, but not by way of limitation, antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, etc. Additionally, the derivative can contain one or more non-classical amino acids.

The MT1 and/or MT2 inhibiting antibodies and antigen-binding fragments thereof described herein for use in inhibiting an autoimmune disease by inhibiting differentiation and/or proliferation of Tr1 cells can be generated by any suitable method known in the art. Monoclonal and polyclonal antibodies against, for example, MT1 and/or MT2, their subunits, are known in the art. To the extent necessary, e.g., to generate antibodies with particular characteristics or epitope specificity, the skilled artisan can generate new monoclonal or polyclonal MT1 and/or MT2 antagonist and/or new as briefly discussed herein or as known in the art.

Polyclonal antibodies specific for MT1 and/or MT2 can be produced by various procedures well known in the art. For example, MT1 and/or MT2 subunit polypeptides or fragments thereof of SEQ ID NO:1-27 polypeptides or fragments thereof of any of SEQ ID NO:1 to 27, can be administered to various host animals including, but not limited to, rabbits, mice, rats, etc. to induce the production of sera containing polyclonal antibodies specific for the protein. Polyclonal antibodies are preferably raised in animals by multiple subcutaneous (sc) or intraperitoneal (ip) injections of the relevant antigen and an adjuvant. It can be useful to conjugate the antigen to a protein that is immunogenic in the species to be immunized, e.g., keyhole limpet hemocyanin, serum albumin, bovine thyroglobulin, or soy-bean trypsin inhibitor using a bifunctional or derivatizing agent, for example, maleimidobenzoyl sulfosuccinimide ester (conjugation through cysteine residues), N-hydroxy-succinimide (through lysine residues), glutaraldehyde, succinic anhydride, SOCl₂, or R ¹N═C═NR, where R and R¹ are different alkyl groups. Various other adjuvants can be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Suitable adjuvants are also well known to one of skill in the art. In some embodiments, peptide fragment for production of antibodies to MT1 and/or MT2 are disclosed in US application 2010/0166759, which is incorporated herein in its entirety by reference.

Monoclonal antibodies can be prepared using a wide variety of techniques known in the art including the use of hybridoma, recombinant, and phage display technologies, or a combination thereof. Various methods for making monoclonal antibodies described herein are available in the art. For example, the monoclonal antibodies can be made using the hybridoma method first described by Kohler et al., Nature, 256:495 (1975), or any later developments thereof, or by recombinant DNA methods (U.S. Pat. No. 4,816,567). For example, monoclonal antibodies can be produced using hybridoma techniques including those known in the art and taught, for example, in Harlow et al., Antibodies: A Laboratory Manual, (Cold Spring Harbor Laboratory Press, 2nd ed., 1988); Hammerling, et al., in: Monoclonal Antibodies and T-Cell Hybridomas 563-681 (Elsevier, N.Y., 1981) (said references incorporated by reference in their entireties). Methods for producing and screening for specific antibodies using hybridoma technology are routine and well known in the art. In another example, antibodies useful in the methods and compositions described herein can also be generated using various phage display methods known in the art, such as isolation from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

Human antibodies can be made by a variety of methods known in the art, including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741, the contents of which are herein incorporated by reference in their entireties.

Human antibodies can also be produced using transgenic mice which express human immunoglobulin genes, and upon immunization are capable of producing a full repertoire of human antibodies in the absence of endogenous immunoglobulin production. For an overview of this technology for producing human antibodies, see, Lonberg and Huszar, 1995, Int. Rev. Immunol. 13:65-93. For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, the contents of which are herein incorporated by reference in their entireties. In addition, companies such as Abgenix, Inc. (Freemont, Calif.) and Medarex (Princeton, N.J.) can be engaged to provide human antibodies directed against a selected antigen using technology similar to that described above. See also, e.g., Jakobovits et al., Proc. Natl. Acad. Sci. USA, 90:2551 (1993); Jakobovits et al., Nature, 362:255-258 (1993); Bruggermann et al., Year in Immuno, 7:33 (1993); and Duchosal et al. Nature 355:258 (1992), the contents of which are herein incorporated by reference in their entireties. Alternatively, phage display technology (McCafferty et al., Nature 348:552-553 (1990)) can be used to produce human antibodies and antibody fragments in vitro, from immunoglobulin variable (V) domain gene repertoires from unimmunized donors. Human antibodies can also be generated by in vitro activated B cells (see U.S. Pat. Nos. 5,567,610 and 5,229,275, the contents of which are herein incorporated by reference in their entireties). Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope (Jespers et al., 1994, Bio/technology 12:899-903).

As used herein, a “blocking” antibody or an antibody “antagonist” is one which inhibits or reduces biological activity of the antigen(s) it binds. For example, a MT1 and/or MT2 antagonist antibody can bind MT1 and/or MT2 and inhibit the ability of MT1 and/or MT2 inhibit IL-10 production from Tr1 regulatory T-cells, and/or promote the differentiation and/or proliferation of Tr1 regulatory T cells. In certain embodiments, the blocking antibodies (also called neutralizing antibodies) or antagonist antibodies or fragments thereof described herein completely or partially inhibit the biological activity of the antigen(s), e.g., MT1 and/or MT2.

“An “Fv” fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the V_{H}-V_{L} dimer Collectively, the six CDRs or a subset thereof confer antigen binding specificity to the antibody. However, even a single variable domain (or half of an Fv comprising only three CDRs specific for an antigen) has the ability to recognize and bind antigen, although usually at a lower affinity than the entire binding site.

As used herein, “antibody variable domain” refers to the portions of the light and heavy chains of antibody molecules that include amino acid sequences of Complementarity Determining Regions (CDRs; i.e., CDR1, CDR2, and CDR3), and Framework Regions (FRs). V_{H} refers to the variable domain of the heavy chain. V_{L} refers to the variable domain of the light chain. According to the methods used in this invention, the amino acid positions assigned to CDRs and FRs may be defined according to Kabat (Sequences of Proteins of Immunological Interest (National Institutes of Health, Bethesda, Md., 1987 and 1991)). Amino acid numbering of antibodies or antigen binding fragments is also according to that of Kabat.

As used herein, the term “Complementarity Determining Regions” (CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (i.e. about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop. For example, the CDRH1 of the human heavy chain of antibody 4D5 includes amino acids 26 to 35.

“Framework regions” (hereinafter FR) are those variable domain residues other than the CDR residues. Each variable domain typically has four FRs identified as FR1, FR2, FR3 and FR4. If the CDRs are defined according to Kabat, the light chain FR residues are positioned at about residues 1-23 (LCFR1), 35-49 (LCFR2), 57-88 (LCFR3), and 98-107 (LCFR4) and the heavy chain FR residues are positioned about at residues 1-30 (HCFR1), 36-49 (HCFR2), 66-94 (HCFR3), and 103-113 (HCFR4) in the heavy chain residues. If the CDRs comprise amino acid residues from hypervariable loops, the light chain FR residues are positioned about at residues 1-25 (LCFR1), 33-49 (LCFR2), 53-90 (LCFR3), and 97-107 (LCFR4) in the light chain and the heavy chain FR residues are positioned about at residues 1-25 (HCFR1), 33-52 (HCFR2), 56-95 (HCFR3), and 102-113 (HCFR4) in the heavy chain residues. In some instances, when the CDR comprises amino acids from both a CDR as defined by Kabat and those of a hypervariable loop, the FR residues will be adjusted accordingly. For example, when CDRH1 includes amino acids H26-H35, the heavy chain FR1 residues are at positions 1-25 and the FR2 residues are at positions 36-49.

As used herein, a “chimeric antibody” refers to a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science, 1985, 229:1202; Oi et al, 1986, Bio-Techniques 4:214; Gillies et al., 1989, J. Immunol Methods 125:191-202; U.S. Pat. Nos. 5,807,715; 4,816,567; and 4,816,397, the contents of which are herein incorporated by reference in their entireties.

“Humanized antibodies,” as the term is used herein, refer to antibody molecules from a non-human species, where the antibodies that bind the desired antigen, i.e., MT1 and/or MT2, have one or more CDRs from the non-human species, and framework and constant regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., 1988, Nature 332:323. Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology, 1991, 28(4/5):489-498; Studnicka et al., 1994, Protein Engineering 7(6):805-814; Roguska. et al, 1994, PNAS 91:969-973), and chain shuffling (U.S. Pat. No. 5,565,332), the contents of which are herein incorporated by reference in their entireties. Accordingly, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), the contents of which are herein incorporated by reference in their entireties, by substituting rodent CDRs or CDR sequences for the corresponding sequences of a human antibody. Accordingly, such “humanized” antibodies are chimeric antibodies (U.S. Pat. No. 4,816,567, the contents of which are herein incorporated by reference in its entirety) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The “Fab” fragment contains a variable and constant domain of the light chain and a variable domain and the first constant domain (C_{H}1) of the heavy chain. F(ab′)₂ antibody fragments comprise a pair of Fab fragments which are generally covalently linked near their carboxy termini by hinge cysteines between them. Other chemical couplings of antibody fragments are also known in the art.

“Single-chain Fv” or “scFv” antibody fragments comprise the V_{H} and V_{L} domains of antibody, wherein these domains are present in a single polypeptide chain. Generally the Fv polypeptide further comprises a polypeptide linker between the V_{H} and V_{L} domains, which enables the scFv to form the desired structure for antigen binding. For a review of scFv, see Pluckthun in The Pharmacology of Monoclonal Antibodies, Vol 113, Rosenburg and Moore eds. Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H} and V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The expression “linear antibodies” refers to the antibodies described in Zapata et al., Protein Eng., 8(10):1057-1062 (1995). Briefly, these antibodies comprise a pair of tandem Fd segments (V_{H}-C_{H}1-V_{H}-C_{H}1) which, together with complementary light chain polypeptides, form a pair of antigen binding regions. Linear antibodies can be bispecific or monospecific.

Various techniques have been developed for the production of antibody or antigen-binding fragments. The antibodies described herein can be fragmented using conventional techniques and the fragments screened for utility in the same manner as described above for the whole antibodies. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). For example, Fab and F(ab′)₂ fragments of the bispecific and multispecific antibodies described herein can be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the C_{H}1 domain of the heavy chain. However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185.

Examples of techniques which can be used to produce single-chain Fvs and antibodies include those described in U.S. Pat. Nos. 4,946,778 and 5,258,498; Huston et al., 1991, Methods in Enzymology 203:46-88; Shu et al., 1993, PNAS 90:7995-7999; and Skerra et al., 1988, Science 240:1038-1040. For some uses, including the in vivo use of antibodies in humans as described herein and in vitro proliferation or cytotoxicity assays, it is preferable to use chimeric, humanized, or human antibodies.

An “affinity matured” antibody is one with one or more alterations in one or more CDRs thereof which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by V_{H} and V_{L} domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol 154(7):3310-9 (1995); and Hawkins et al., J. Mol. Biol. 226:889-896 (1992).

As used herein “complementary” refers to when two immunoglobulin domains belong to families of structures which form cognate pairs or groups or are derived from such families and retain this feature. For example, a V_{H} domain and a V_{L} domain of a natural antibody are complementary; two V_{H} domains are not complementary, and two V_{L} domains are not complementary. Complementary domains can be found in other members of the immunoglobulin superfamily, such as the V_{α} and V_{β} (or γ and δ) domains of the T-cell receptor. Domains which are artificial, such as domains based on protein scaffolds which do not bind epitopes unless engineered to do so, are non-complementary. Likewise, two domains based on, for example, an immunoglobulin domain and a fibronectin domain are not complementary.

The process of designing/selecting and/or preparing a bispecific or multispecific polypeptide agent as described herein, is also referred to herein as “formatting” the amino acid sequence, and an amino acid sequence that is made part of a bispecific or multispecific polypeptide agent described herein is said to be “formatted” or to be “in the format of” that bispecific or multispecific polypeptide agent. Examples of ways in which an amino acid sequence can be formatted and examples of such formats will be clear to the skilled person based on the disclosure herein; and such formatted amino acid sequences form a further aspect of the bispecific or multispecific polypeptide agents described herein.

The term “library,” as used herein, refers to a mixture of heterogeneous polypeptides or nucleic acids. The library is composed of members, each of which have a single polypeptide or nucleic acid sequence. To this extent, library is synonymous with repertoire. Sequence differences between library members are responsible for the diversity present in the library. The library can take the form of a simple mixture of polypeptides or nucleic acids, or can be in the form of organisms or cells, for example bacteria, viruses, animal or plant cells and the like, transformed with a library of nucleic acids. Preferably, each individual organism or cell contains only one or a limited number of library members. Advantageously, the nucleic acids are incorporated into expression vectors, in order to allow expression of the polypeptides encoded by the nucleic acids. In a preferred aspect, therefore, a library can take the form of a population of host organisms, each organism containing one or more copies of an expression vector containing a single member of the library in nucleic acid form which can be expressed to produce its corresponding polypeptide member. Thus, the population of host organisms has the potential to encode a large repertoire of genetically diverse polypeptide variants.

As used herein, the term “comprising” means that other elements can also be present in addition to the defined elements presented. The use of “comprising” indicates inclusion rather than limitation.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean ±1%.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

All patents and other publications identified are expressly incorporated herein by reference for the purpose of describing and disclosing, for example, the methodologies described in such publications that could be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

This invention is further illustrated by the following examples which should not be construed as limiting.

### EXAMPLES

IL-27 induces type 1 regulatory T (Tr1) cells suppress autoimmunity by producing IL-10. STAT1 and STAT3 have been described as key transcription factors that promote IL-10 secretion from Tr1 cells induced by IL-27. However, the molecular pathways for negatively regulating Tr1 cell differentiation remain elusive. Here, the inventors demonstrate that IL-27 induces metallothioneins (MTs) that in turn prevent Tr1 cell development. MT expression leads to the reduction of STAT1 and STAT3 phosphorylation under Tr1 differentiation condition, resulting in impaired IL-10 production. Accordingly, Tr1 cells derived from MT-deficient mice showed an increased ability to produce IL-10 and potently suppress Experimental Autoimmne Encephalomyelitis (EAE) upon adoptive transfer. Moreover, activation of STAT1 and/or STAT3 can overcome the suppression of IL-10 by MTs, indicating a dynamic balance between STATs and MTs in regulating IL-10 during Tr1 cell differentiation.

Materials and Methods

Animals

Mt^{-/-} mice (129/Sv-MT1MT2^{tml bri}), 129/Sv (control), 129/Sv B6 F1, 2D2, Stat1^{-/-} and C57BL/6 mice were purchased from Jackson Laboratory (Bar Harbor, Me., USA). Stat3^{-/-} mice were a kind gift from John O'Shea.

In Vitro T Cell Differentiation

Naïve (CD44^{lo}CD62L⁺CD25⁻) or memory (CD44^{hi}CD62L^{lo}CD25⁻) CD4⁺ T cells was obtained from spleens and lymph nodes of WT or Mt^{-/-} mice by flow cytometry accordingly. The purity of isolated T cell populations routinely exceeded 98%. Naïve T cells were stimulated with plate-bound anti-CD3 (145-2C11, 1 μg/ml) and anti-CD28 (PV-1, 1 μg/ml). Murine recombinant IL-27 (50 ng/ml) was purchased from R&D Systems.

Human Tr1 Differentiation

FACS sorted human naïve (CD45RO⁻CD62L⁺) CD4⁺ T cells were cultured in the presence of plate bound anti-CD3 and anti-CD28 (2 μg/ml) and IL-27 (100 ng/ml) in the presence or absence of metallothionin-1 & 2 at indicated doses (1 μM and 5 μM) for 5 days.

Measurement of Cytokines

Secreted cytokines were measured by ELISA at the indicated times. For intracellular cytokine staining, cells were cultured as described above and stimulated for 4 hours at 37° C. in culture medium containing PMA (50 ng/ml; Sigma), ionomycin (1 μg/ml; Sigma) and monensin (GolgiStop; 1 μl/ml; BD Biosciences). After staining for surface markers, cells were fixed and permeabilized according to the manufacturer's instructions (BD Biosciences). All cytokines antibodies were purchased from Biolegend.

Phospho-STAT Staining for FACS

CD4⁺ T cells from WT or Mt^{-/-} mice were either left unstimulated or stimulated with IL-27 for 20 min. Levels of phosphorylated proteins were detected by staining with anti-pSTAT3 (pY705), anti-pSTAT1 (pY701) and anti-pSTAT4 (pY693) (all from BD Biosciences) following the manufacturer's instructions.

Quantitative RT-PCR

RNA was extracted with RNAeasy minikits (Qiagen) and RNA expression levels were analyzed by RT-PCR according to the manufacturer's instructions using the GeneAmp 7500 Sequence Detection System or the ViiA7 Real-Time PCR System (Applied Biosystems). Expression was normalized to the expression of β-actin. Primers-probe mixtures were purchased from Applied Biosystems: Ahr (Mm00478930-ml); Ifng (Mm99999071-ml); Il10 (Mm00439615-g1); Il21 (Mm00517640-ml); Il27ra (Mm00497259-ml); Mt1 (Mm00496660-g1); Mt2 (Mm00809556-s1); Maf (Mm02581355-S1); Tbx21 (Mm00450960-ml); Actb (Mm00446968-ml); Mt1e (Hs01938284_g1); Mt1h (Hs00823168_g1).

Bone Marrow (BM) Chimeric Mice

WT or Mt^{-/-} BM cells were transferred i.v. (10×10⁶ per mouse) to lethally irradiated (11 Gy) WT recipient mice. Mice were used for experiments approximately 6-8 weeks after BM transfer.

In Vivo Treatment with Anti-CD3

Mt^{-/-} and WT chimera mice were treated with 20 μg of anti-CD3 (clone 2C11) or isotype control antibody i.p. every 3 days for a total of 4 times. The mice were sacrificed 4 hours after the last treatment, single cell suspensions were prepared from spleens and CD4⁺ cells were isolated by positive magnetic selection (Miltenyi). RNA was subsequently isolated from purified CD4⁺ T cells and subjected to RT-PCR.

Adoptive Transfer

10 days after immunization of 129/Sv and Mt^{-/-} mice with MOG₃₃₋₃₅-55 peptide, draining lymph node cells were isolated and seeded into 12-well tissue culture plates (10×10⁶ cells/ml, 2 ml/well). Cells were stimulated with 20 μg/ml MOG₃₅₋₅₅peptide, 10 ng/ml IL-23 and 10 ng/ml IL-12 for effector cells or 30 ng/ml IL-27 for Tr1 cells for 4 days prior to transfer to recipient mice. 20×10⁶ effector cells/mouse were transferred intravenously to 129/Sv B6 F1 recipient mice. For the co-transfers, WT or Mt^{-/-} Tr1 cells were mixed with WT effector T cells at a ratio of 1:3. At days 0 and 2 after transfer mice were injected i.p. with pertussis toxin (20 ng/mouse).

To generate MOG-specific effector T cells 2D2 transgenic CD4⁺ T cells were stimulated with MOG₃₅₋₅₅ peptide, IL-12 (10 ng/ml) and IL-23 (10 ng/ml) in vitro. Tr1 cells were generated by stimulating 2D2 CD4⁺ cells with MOG₃₅-55 peptide and IL-27 (30 ng/ml). Tr1 cells were transduced 24 h after stimulation with either control or MT1-expressing retrovirus. An additional 3 d after transduction, control or MT1 over-expressing GFP⁺ Tr1 cells were FACS sorted and 2×10⁶ cells were co-transferred into C57BL/6 recipients with 2D2 effector T cells (6×10⁶).

CFSE

CFSE-labeled effector T cells (20×10⁶) were transferred to F1 mice with or without WT or Mt^{-/-} Tr1 cells at a ratio of 3:1. At 4 and 7 days after transfer lymph nodes were harvested and CFSE⁺CD4⁺ cells were examined by FACS.

Plasmids

Murine Mt1 and Mt2 (IMAGE cDNA clone) were cloned into the MSCV-IRES-GFP and MSCV-IRES-Thy1.1 vectors at EcoRI/XhoI and BglII/XhoI sites, respectively.

Retroviral Transduction

Retroviral particles were produced by transiently transfecting HEK 293T cells with retroviral packaging constructs and expression plasmids MSCV-IRES-GFP or MSCV-IRES-GFP-MT1/MT2 using Fugene HD (Roche). 72 hours after transfection, viral culture supernatants were harvested, supplemented with polybrene (8 mg/ml) and added to previously stimulated T cells (5×10⁵/well, plate-bound anti-CD3/CD28 and IL-27 for 24 h). Cultures were centrifuged at 800 G for 45 min at 25° C.

Statistical Analysis

Statistical analysis was performed using Prism software (Graph Pad software, La Jolla, Calif., USA). P values<0.05 were considered significant.

### Example 1

### Late Expression of Metallothioneins (MT) in IL-27-Induced Tr1 Cells

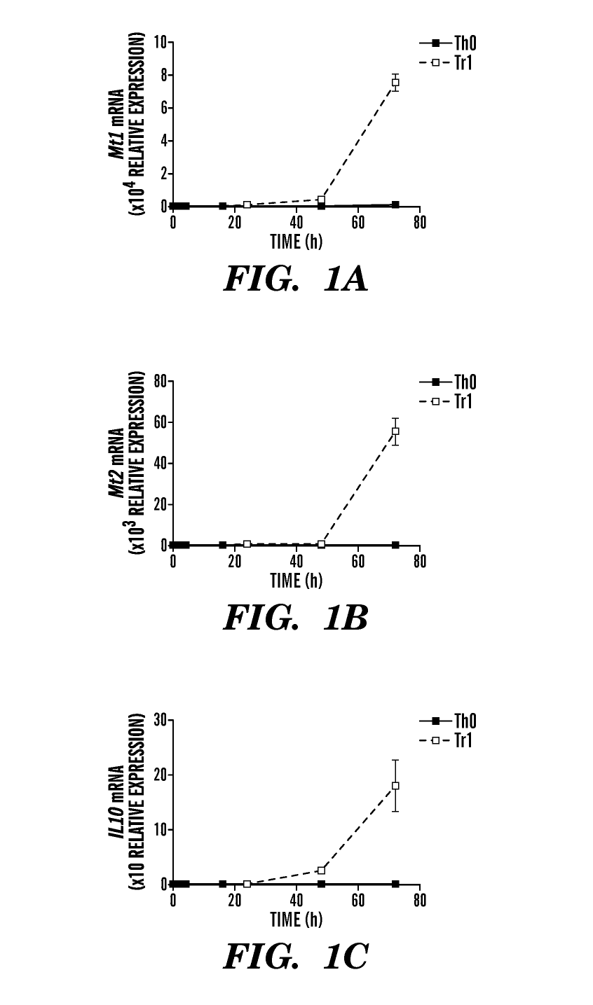

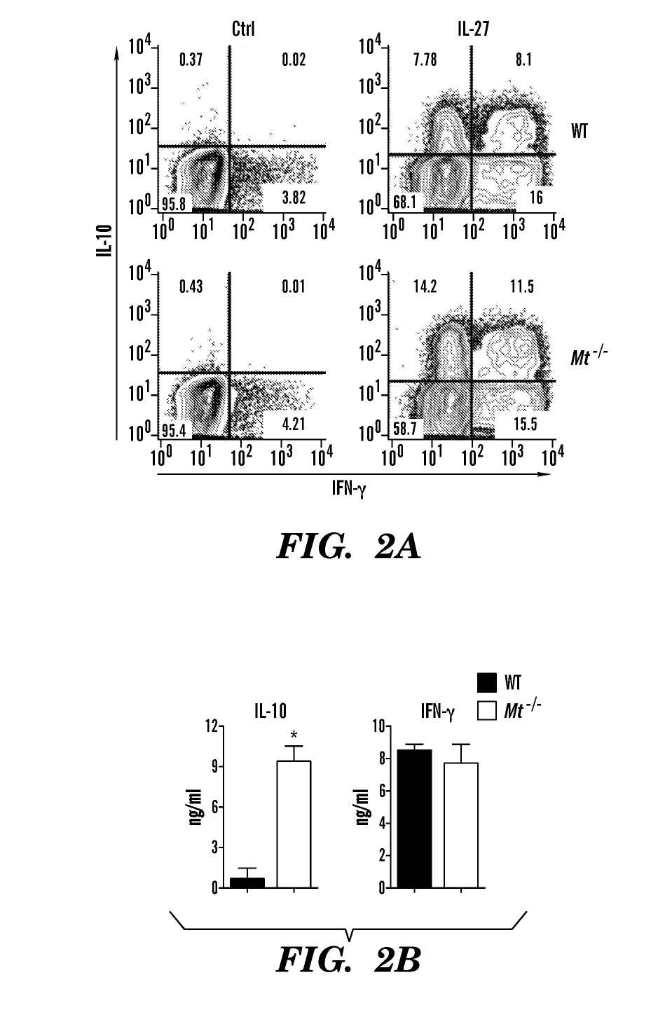

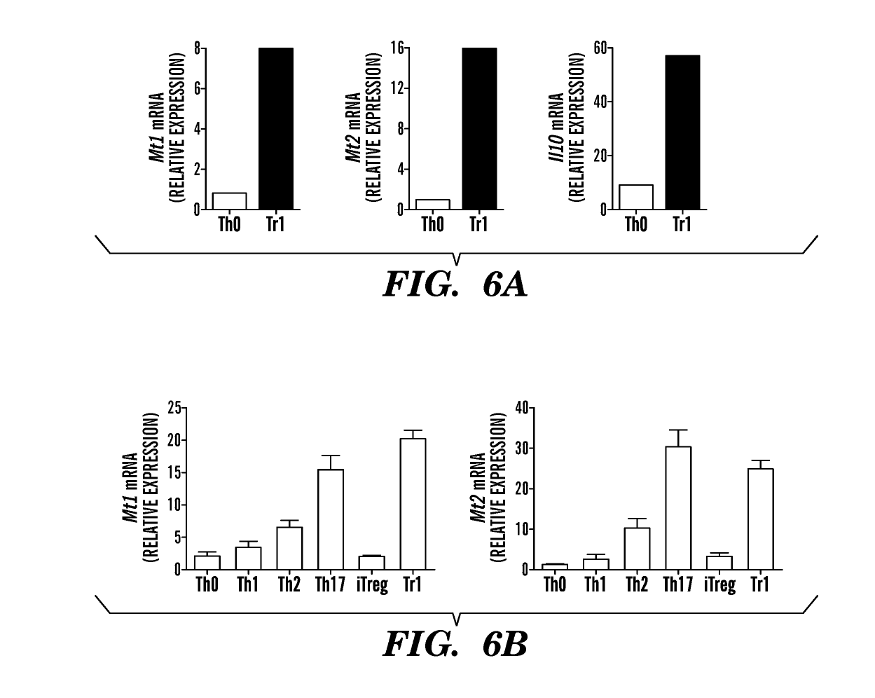

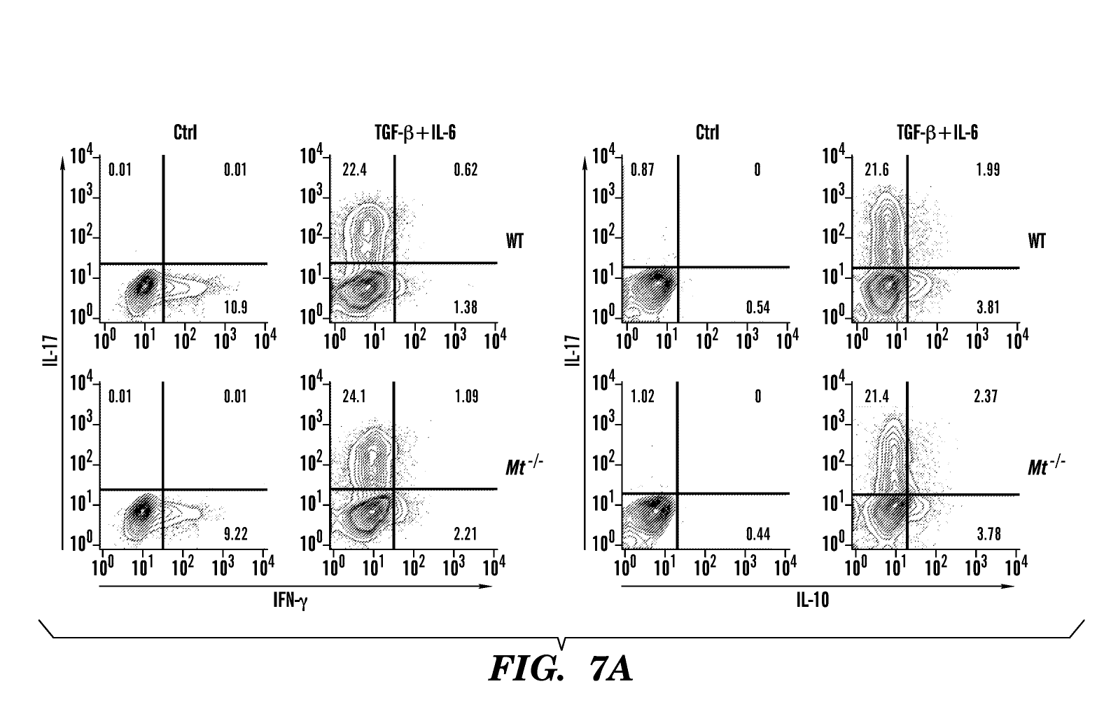

To gain insight into the differentiation of IL-27-induced CD4+ Tr1 cells, the inventors performed a comparative microarray analysis of developing Tr1 cells at 72 hours after stimulation with IL-27. The inventors discovered that metallothioneins (MTs)-1 and 2 were highly expressed in IL-27-induced Tr1 cells generated from naïve CD4⁺CD25⁻CD62L⁺CD44^{low} T cells when compared to T cells similarly activated without the presence of differentiating cytokines (Th0) (FIG. 6A). Next, the inventors then analyzed kinetics of MT1 and MT2 expression during the differentiation of Tr1 cells with IL-27. In contrast to Th0 cells which express only marginal levels of MT1 and 2, both MTs isoforms were highly expressed in IL-27-induced Tr1 cells (FIG. 1). While MTs expression was dramatically enhanced in CD4+ Tr1 cells after 72 hours, there was no significant MTs expression prior to 48 hours. Interestingly, this delayed induction of MTs coincided with the induction of IL-10 in developing Tr1 cells (FIG. 1). The inventors also examined the expression levels of MT1 and MT2 in different subsets of CD4⁺ T cells, and demonstrated that both MTs were highly expressed in Tr1 and Th17 cells whereas Th1 or Th2 cells exhibited modest expression of MT1 and MT2 (FIG. 6B).

### Example 2

### MTs Impair IL-10 Expression in IL-27-Induced Tr1 Cells

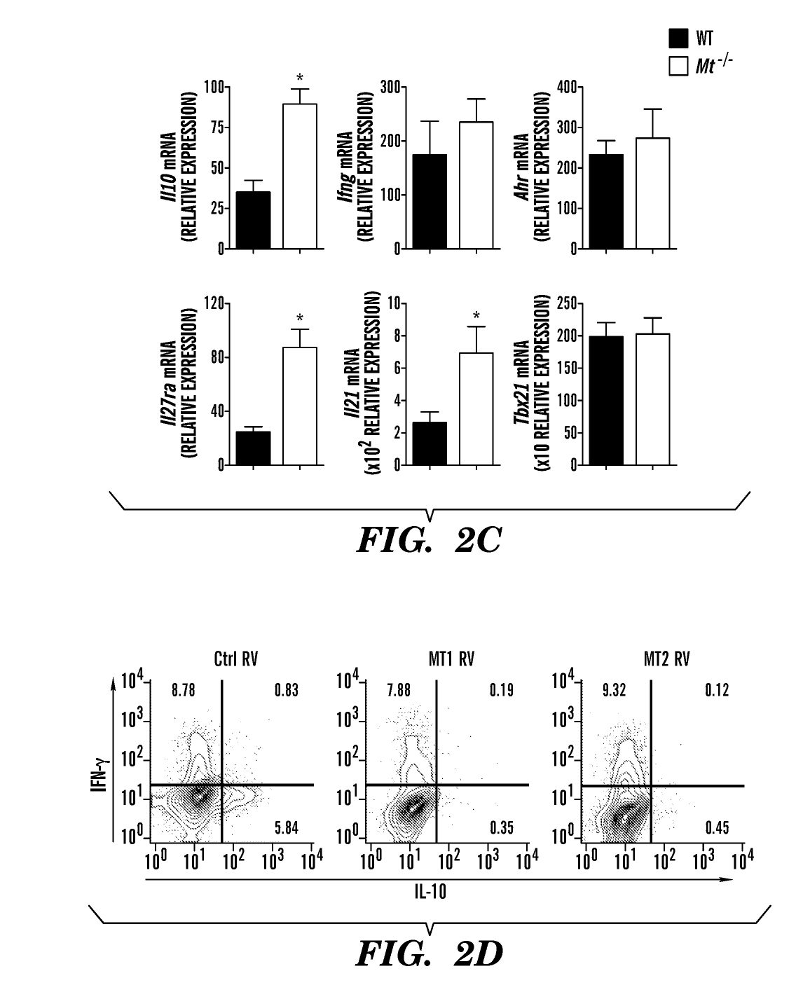

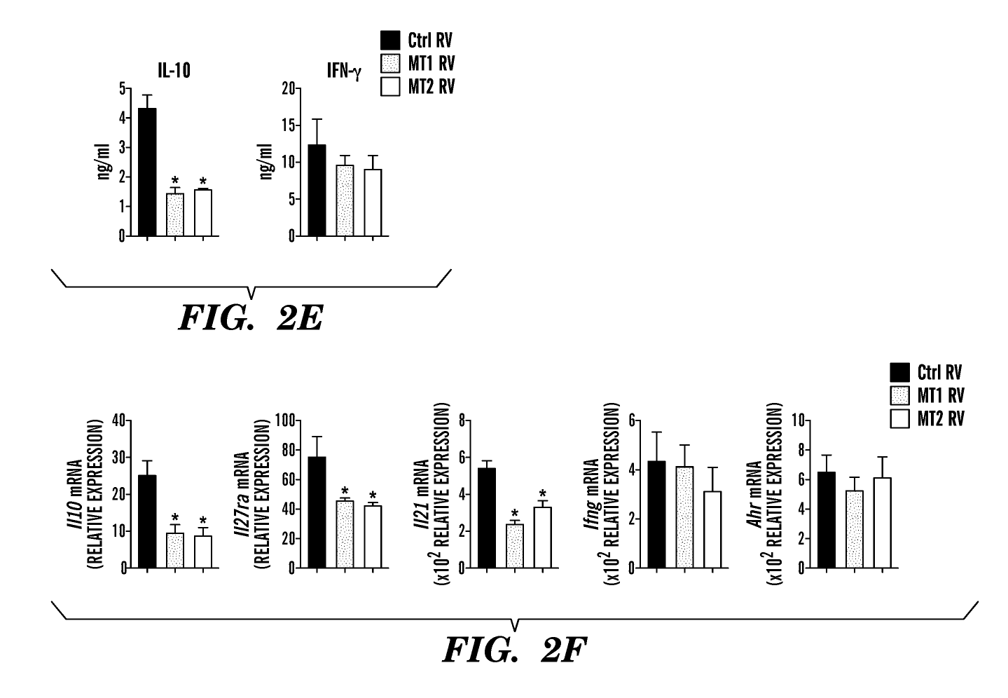

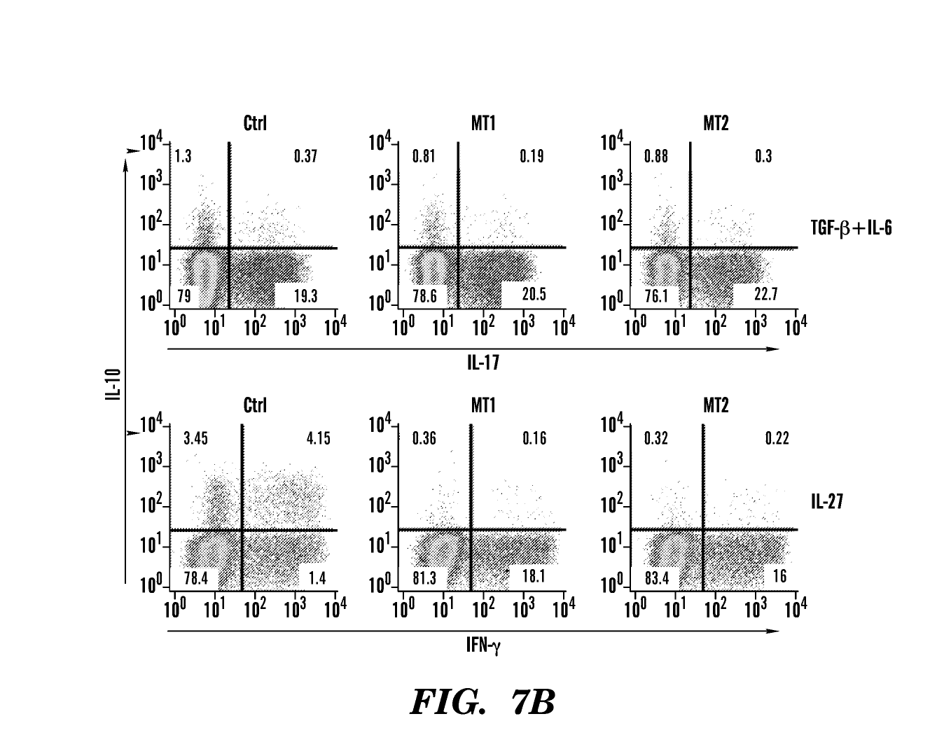

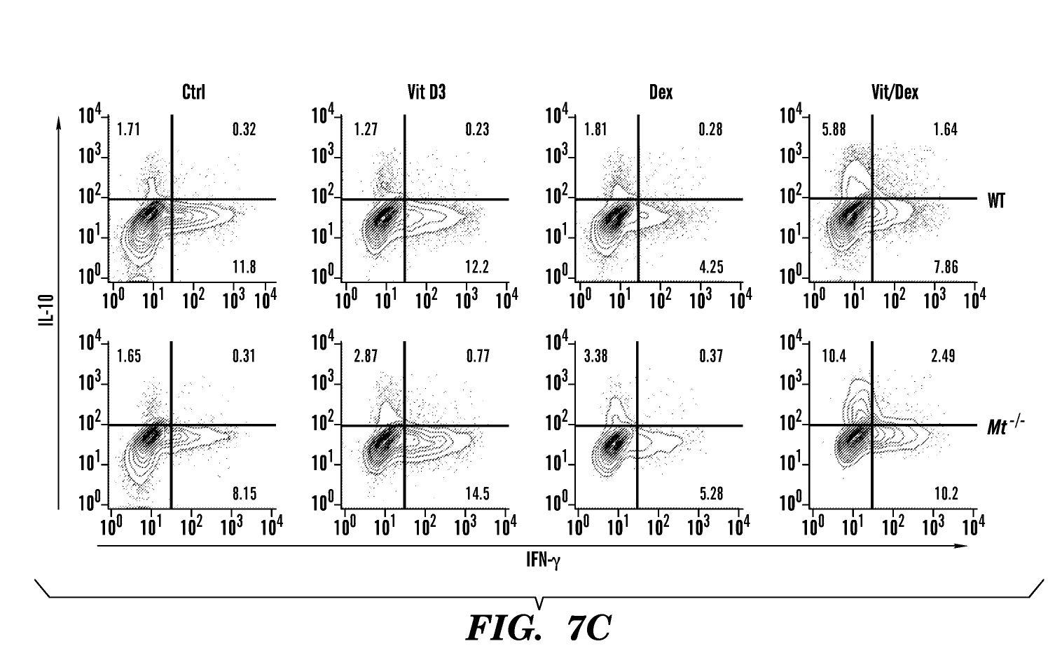

The role of MTs on IL-27-induced Tr1 cells was investigated by differentiating naïve CD4⁺ T cells from WT or Mt^{-/-} mice. While IFN-γ production from Tr1 cells was unaffected in the absence of MTs, the frequency of IL-10-producing cells and the secretion of IL-10 were notably enhanced in Tr1 cells derived from Mt^{-/-} mice (FIG. 2A, 2B). Although MTs are expressed at high levels in Th17 differentiated with TGF-β1 and IL-6, cytokines from Th17 cells including IL-17, IFN-γ and IL-10 were not altered in the loss of MTs (FIG. 7A). Meanwhile, addition of recombinant MT1 or MT2 during differentiation of naïve CD4⁺ T cells with IL-27 severely impaired IL-10 secretion, while it did not affect IFN-γ production. IL-17 secretion from differentiating Th17 cells was not impacted while IL-10 secretion was modestly decreased with MT1 or MT2 (FIG. 7B). Analysis of the signature Tr1 cytokines from WT and Mt Tr1 cell cultures showed increased IL-10 and unchanged IFN-γ production, at mRNA level as well. Additionally, both Il21 and Il27r, which are critical for Tr1 cell development (1, 2) were also upregulated in the absence of MTs (FIG. 2C). However, other genes that are also expressed in Tr1 cells, like Ahr and Tbx21 are unchanged in the absence of MTs (FIG. 2C). To assess if MTs play a role in Tr1 cells generated with other stimuli than IL-27, naïve sorted CD4⁺ T cells were differentiated using vitamin D3 and dexamethasone (12). The inventors discovered that the Mt^{-/-} Tr1 exhibited elevated IL-10 production under both Vitamin D3 and dexamethasone stimulation after 72 hours. The enhancement of IL-10 became more profound when these two were combined (FIG. 7C). Altogether, these data indicate that MTs are critical for Tr1 cell but not Th17 cell development.

To determine whether endogenous overexpression of MTs can reverse the phenotype observed in the Mt^{-/-} Tr1 cells, retroviral overexpression of GFP tagged MT1 or MT2 into WT Tr1 cells under IL-27 stimulation was assessed and resulted in reduced expression of IL-10 and no effect on IFN-γ, as detected by intracellular staining and ELISA (FIG. 2D-2E). Furthermore, sorted GFP cells from the MT1 or MT2 retroviral transduced Tr1 cells were examined for the expression of other key genes expressed in Tr1 cells. Consistent with MT-deficient T cells, expression Il10, Il27r and Il21 genes were downregulated while Ahr and Ifng were unchanged by MT1 or MT2 overexpression (FIG. 2F). Thus, the inventors have discovered that MTs negatively regulate Tr1 differentiation by inhibiting IL-10 production, and impairing the IL-27 signaling pathway by repressing Il27r and Il21 expression.

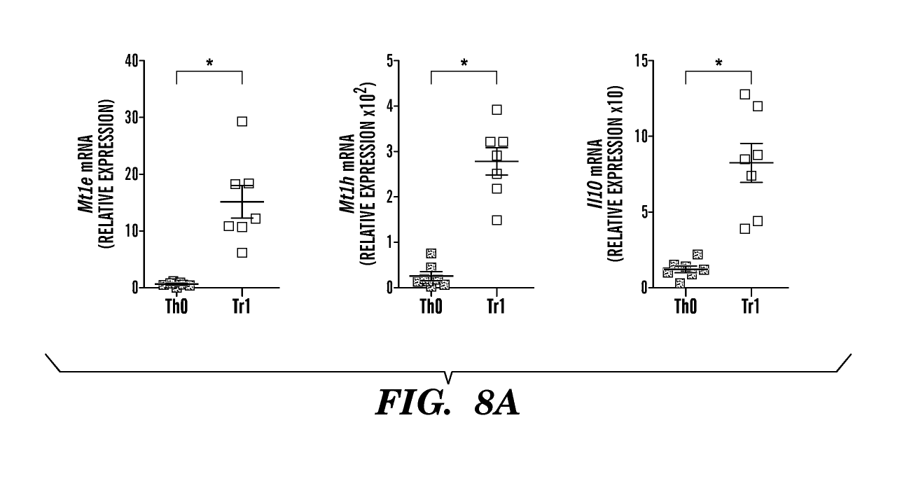

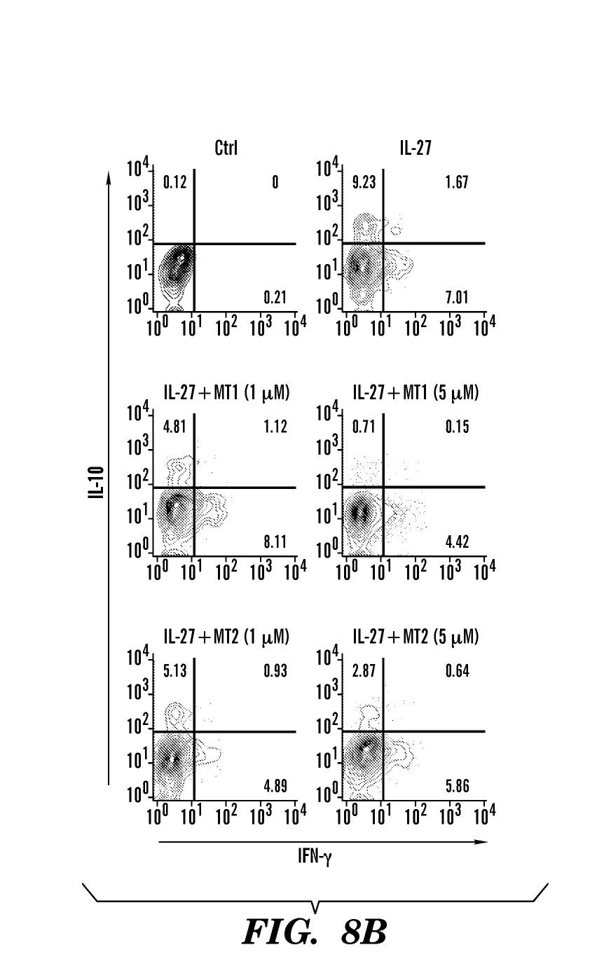

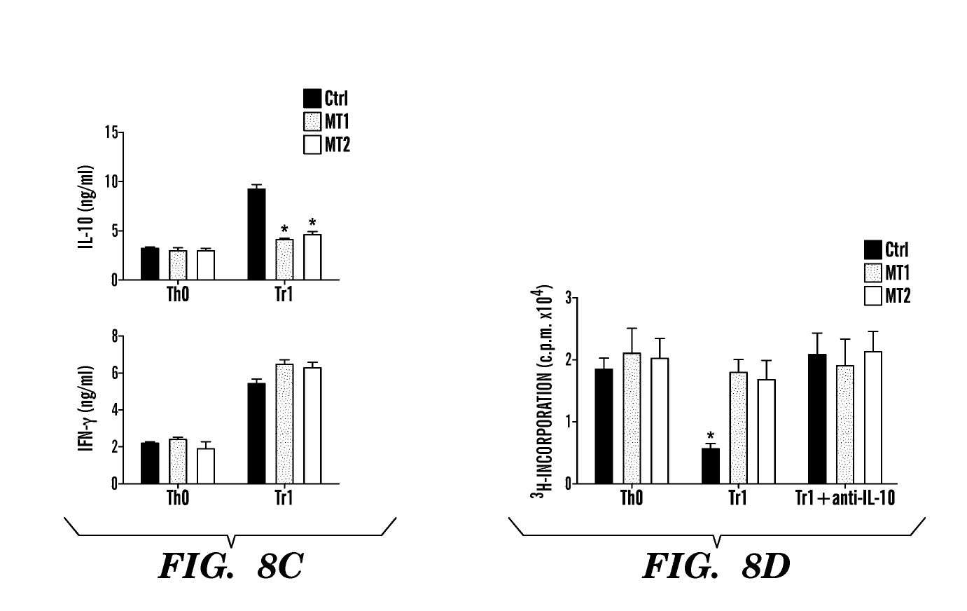

To test if Metallothioneins are also relevant for human Tr1 cell biology, differentiated human Tr1 cell were assessed in vitro to analyze MTs expression and the function of MTs in these cells. In the human genome more than 10 isoforms of MT have been identified compared to only 3 isoforms in the mouse genome. According to NCBI's HomoloGene database human MT1E and MT1H are the closest homologues to murine MT1 and MT2, respectively. The inventors therefore tested the mRNA expression level of these two isoforms in human Tr1 cells. Mt1e and Mt1h were discovered to be highly expressed in human Tr1 cells compared to Th0 cells (FIG. 8A). In order to understand the relevance of MTs in human Tr1 cells, the inventors assessed differentiated human Tr1 cells in the presence of recombinant MT1 and MT2 proteins. Both MT1 and MT2 significantly suppressed the IL-10 production of human Tr1 cells (FIG. 8B-8V). Moreover, the supernatants from cultured Tr1 cells inhibited proliferation of bystander CD4⁺ T cells, while the supernatants from the Tr1 cultivation with additional MT1 or MT2 exhibited compromised suppression capacity in a IL-10 dependent manner (FIG. 8D). Thus the inventors have demonstrated that not only the IL-10 production, but also the inhibitory feature of human Tr1 cells are negatively regulated by MTs.

### Example 3

### MTs Negatively Regulate STAT1 and STAT3 Activation

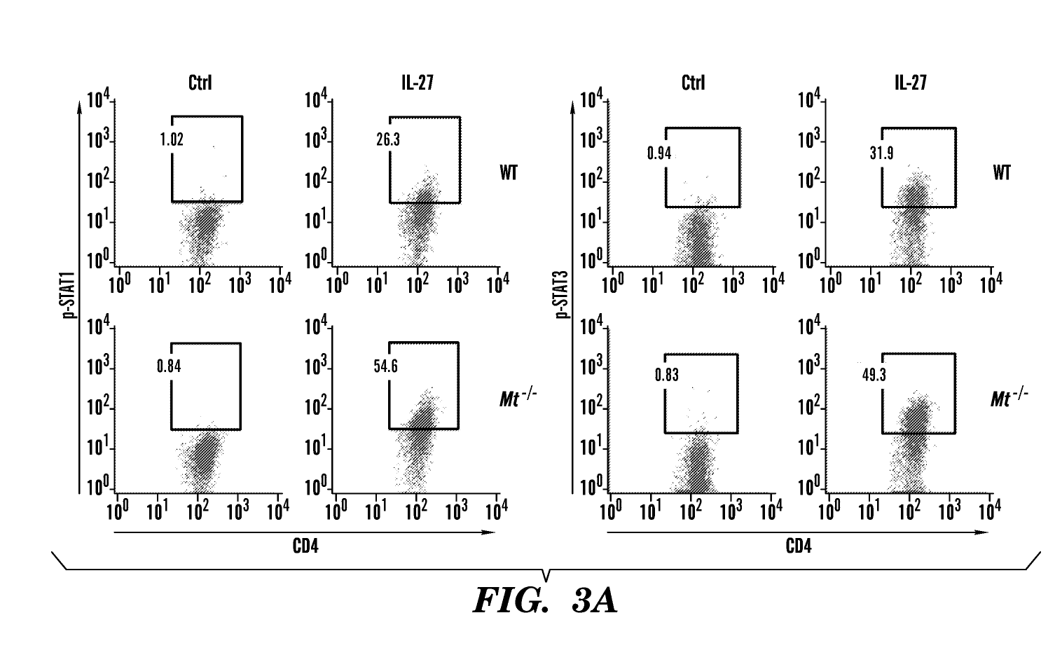

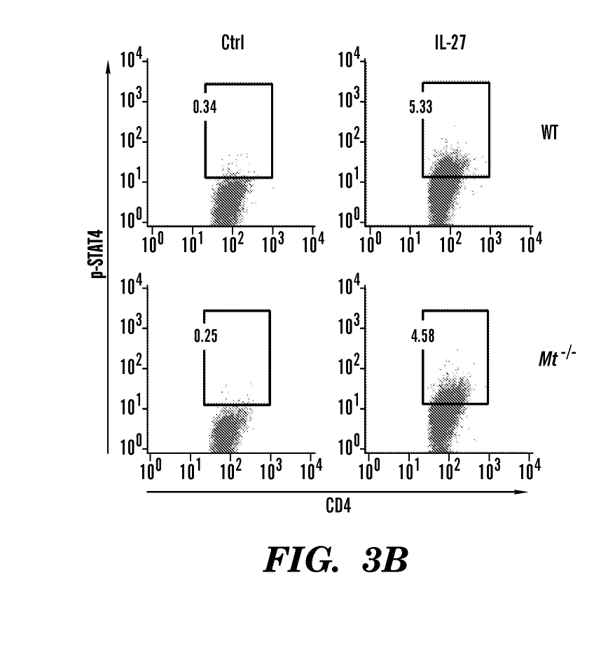

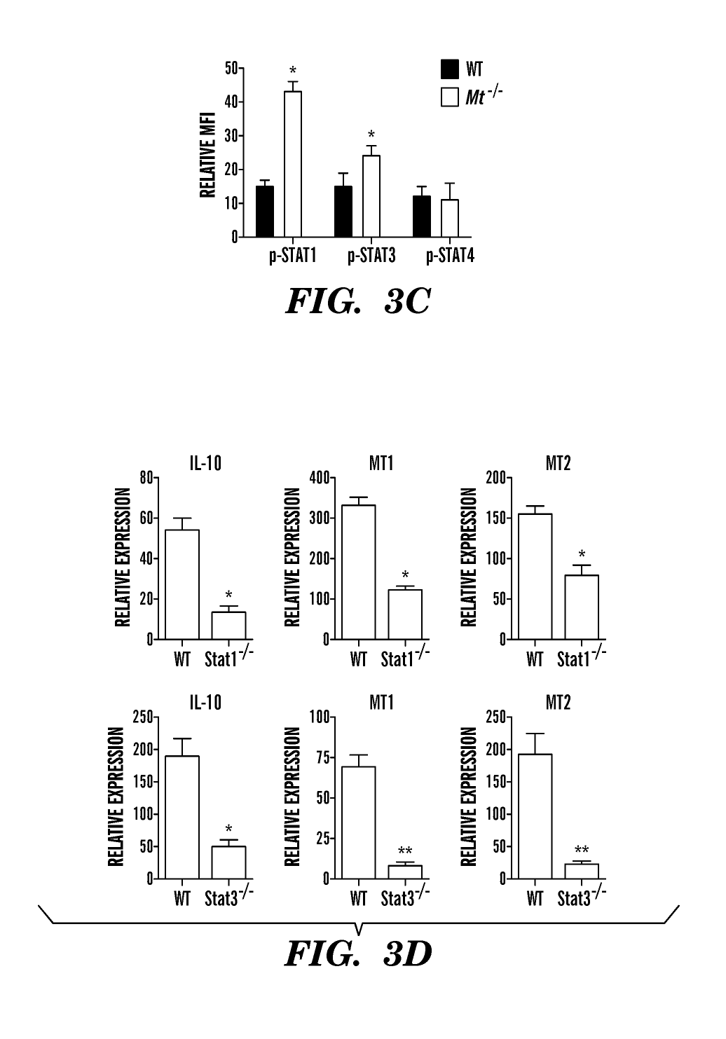

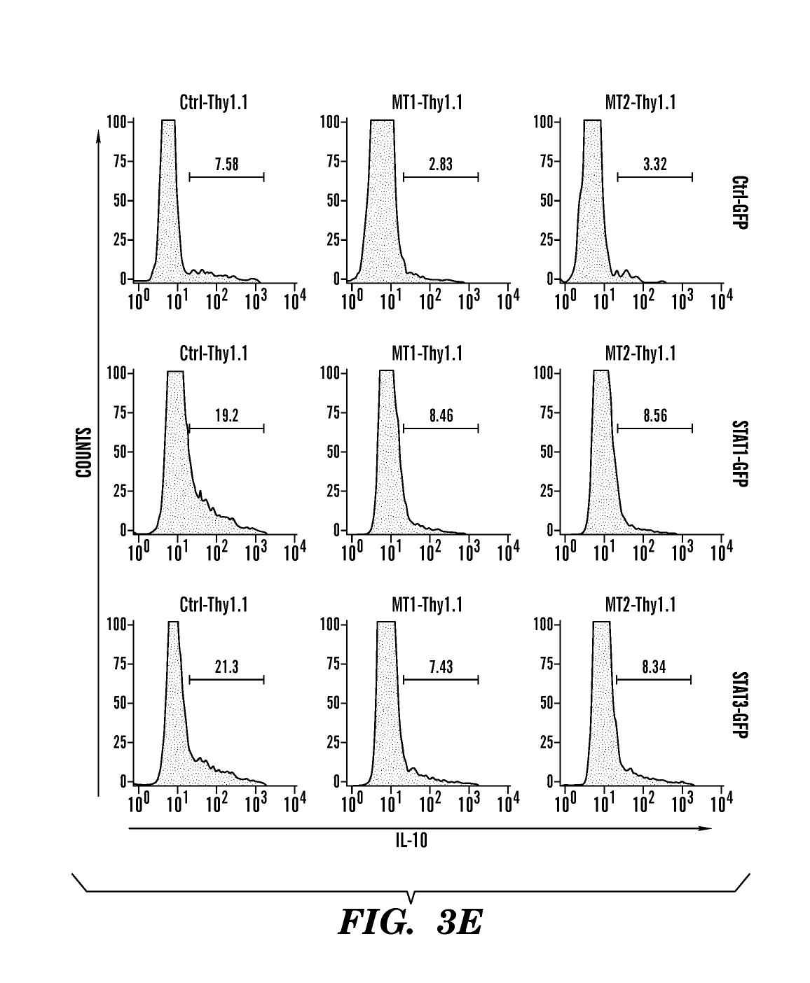

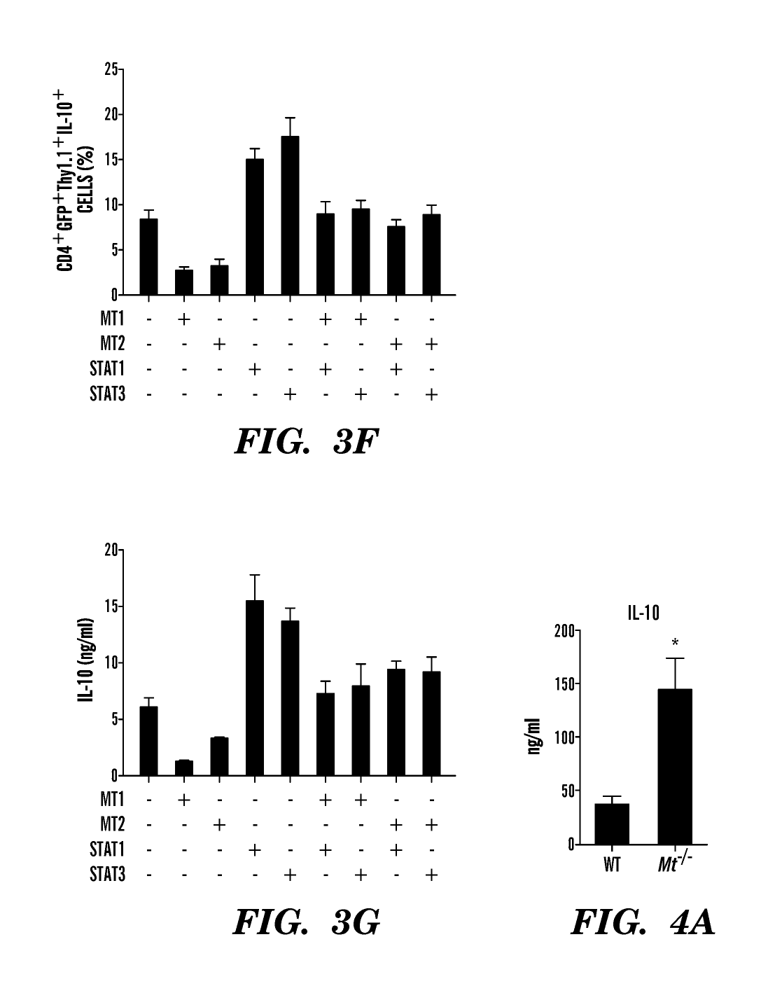

Previous studies have shown that STAT1 and STAT3 are both critical for the induction of IL-10 production in Tr1 cells (6, 13). To assess whether MTs regulate Tr1 differentiation by influencing the activation of STATs, the inventors assessed the activation level of STAT1 and STAT3 after IL-27 stimulation. To this end, naïve CD4⁺ T cells from WT and Mt^{-/-} mice were activated with IL-27 and phosphorylation of STAT1 and STAT3 was analyzed. The inventors discovered that both STAT1 and STAT3 were hyper-phosphorylated upon IL-27 stimulation in Mt^{-/-} compared with WT Tr1-cells (FIGS. 3A and 3C). Besides STAT1 and STAT3, STAT4 is also required for IL-10 production (14), while there was no difference in the pSTAT4 in the WT and Mt^{-/-} Tr1 cells (FIG. 3B-3C). These results demonstrate that MTs inhibit IL-10 production in Tr1 by regulating the activation of STAT1 and STAT3.

In order to identify a potential relationship between STAT1/3 and MTs during Tr1 cell differentiation, the inventors analyzed T cells from Stat1^{-/-} and Stat3^{-/-} mice. Consistent with previous findings, both STAT1 and STAT3 deficient Tr1 cells exhibited reduced Il10 expression. Additionally, both Stat1^{-/-} and Stat3^{-/-} Tr1 cells displayed reduced mRNA expression levels of Mt1 and Mt2 (FIG. 3D). The inventors co-infected naïve Tr1 cells with retroviruses encoding MT1 or MT2 (MIT vector, Thy1.1 reporter) and STAT1 or STAT3 (MIG vector, GFP reporter) and stimulated the cells with IL-27. Over-expression of either STAT1 or STAT3 could reverse the suppressive effect of MT1 or MT2 on IL-10 production (FIG. 3E-3F). These results demonstrate that MTs inhibit Tr1 cell differentiation and potentially compete with positive regulators of IL-10 such as STATs, subsequently defining the STAT-driven threshold for the induction of IL-10. However, the inventors determined that both STAT1 and STAT3 are dominant over MTs in the regulation of IL-10 during Tr1 cell polarization. Thus, expression levels and activation status of STAT1/3 and MTs may form a kinetic balance to control IL-10 production in Tr1 cells.

### Example 4

### MTs Control the Induction of Tr1 Cells In Vivo

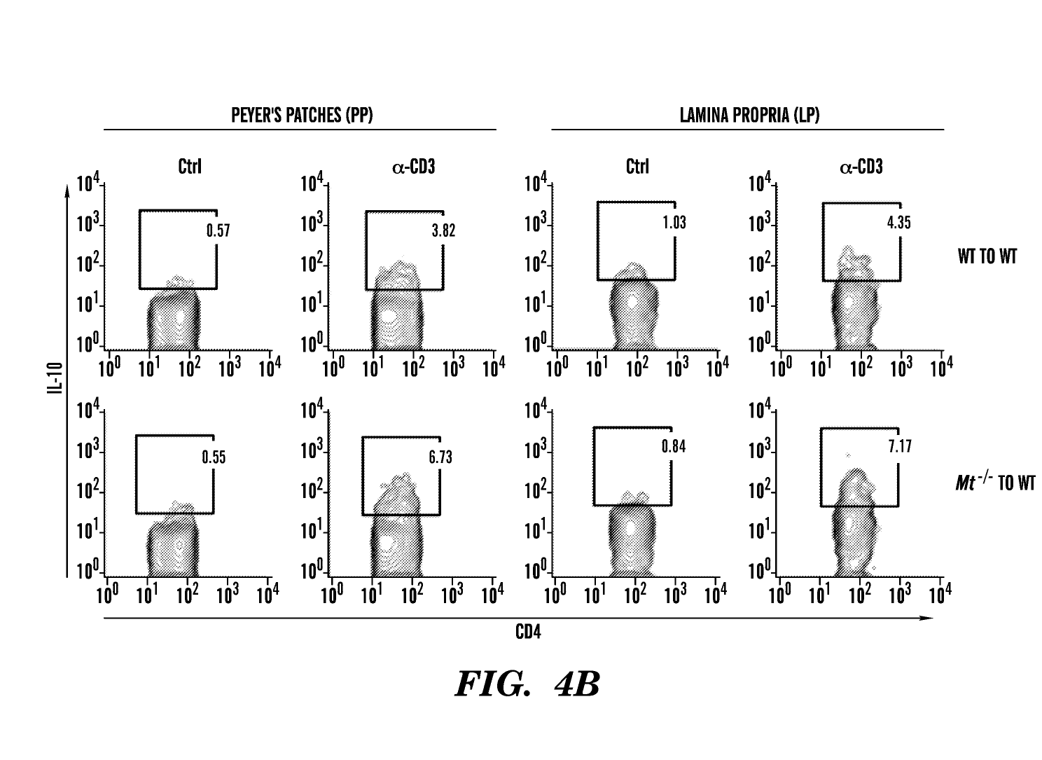

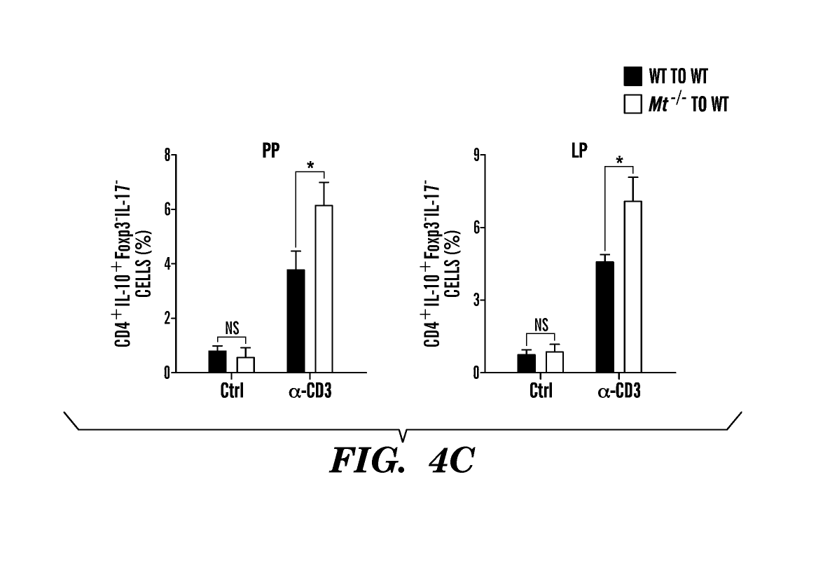

To study the relevance of MTs in expanding Tr1 cells in vivo, IL-10-producing Tr1 cells generated from CD4⁺CD25⁻CD44^{h1}CD62L⁻ memory T cells were examined from WT versus Mt^{-/-} mice. The inventors discovered that IL-10 secretion from memory T cells from Mt^{-/-} mice were increased by over 50% as compared to WT mice (FIG. 4A). It has previously been shown that repeated in vivo treatment with anti-CD3 induces Tr1 cells, which is dependent on IL-27 for their generation (1, 15). Since MT is expressed at other tissues such as liver, it not clear whether the in vivo increase in IL-10 is due to a direct effect on T cells. To exclude the effect from the non-hematopoietic cell derived MTs, the inventors generated bone marrow (BM) chimera in which the WT host were reconstituted with either WT or Mt^{-/-} BM. Eight weeks after reconstitution, an anti-CD3 or an isotype control antibody was repeatedly administrated to the WT and Mt^{-/-} BM chimera mice. After treatment, the Tr1 cell frequency in the peyer's patches (PP) and lamina propria (LP) was analysed. To rule out the confounding effects by other IL-10 producing T cell subsets and specifically examine Tr1 cells, IL-10 production was assessed by gating on CD4⁺IL-17⁻Foxp3⁻ cells. The inventors demonstrated that there was a significant increase in Tr1 cells in Mt^{-/-} BM chimera's PP and LP compared with WT chimeras (FIG. 4B-4Cc). These results further demonstrate that MTs regulate the generation of IL-10⁺Tr1 cells by specifically acting on the haemopoetic compartment in vivo.

Enhanced Suppressive Capacity of IL-10 Producing T Cells in the Absence of MT In Vivo

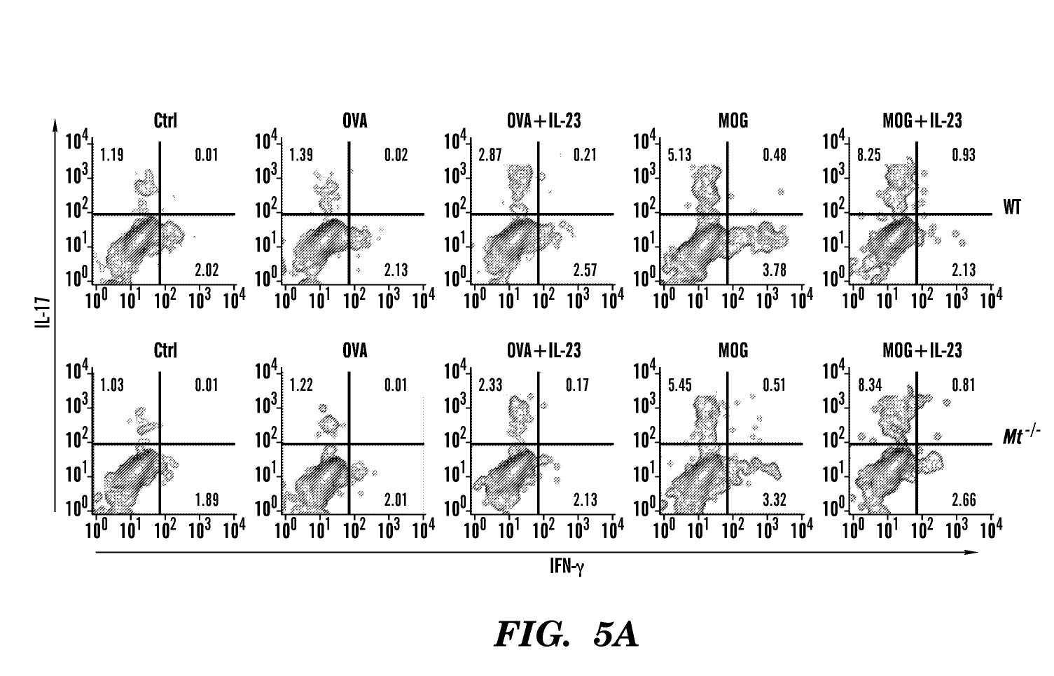

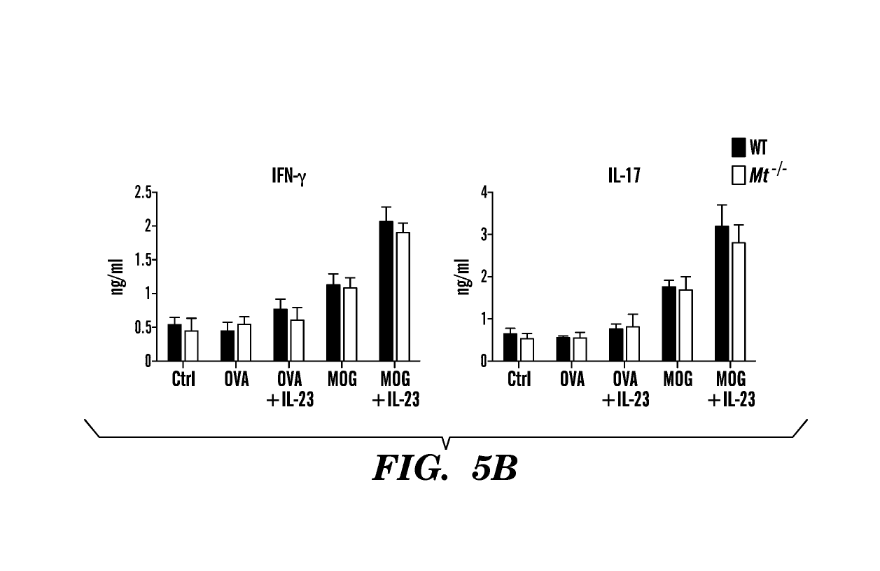

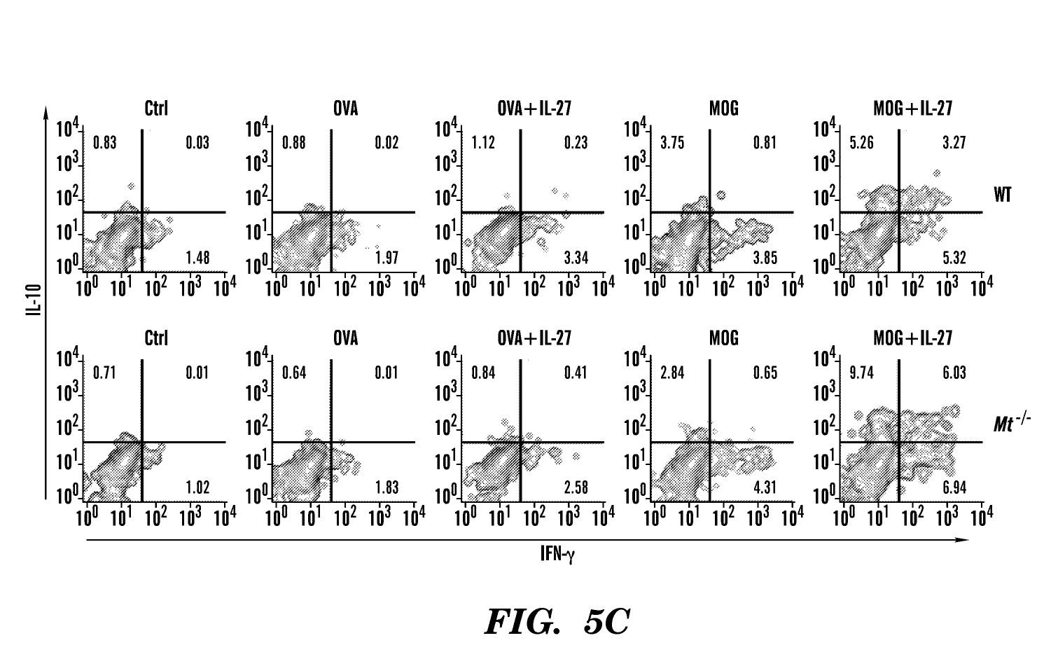

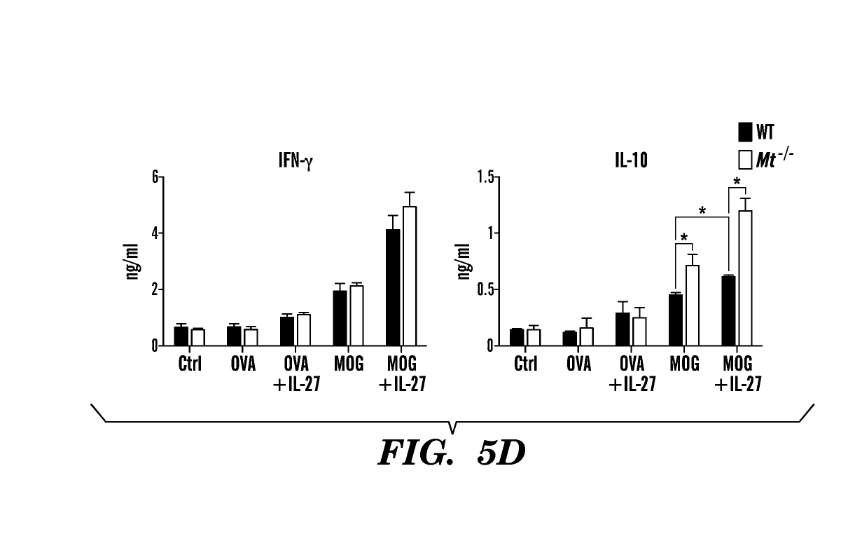

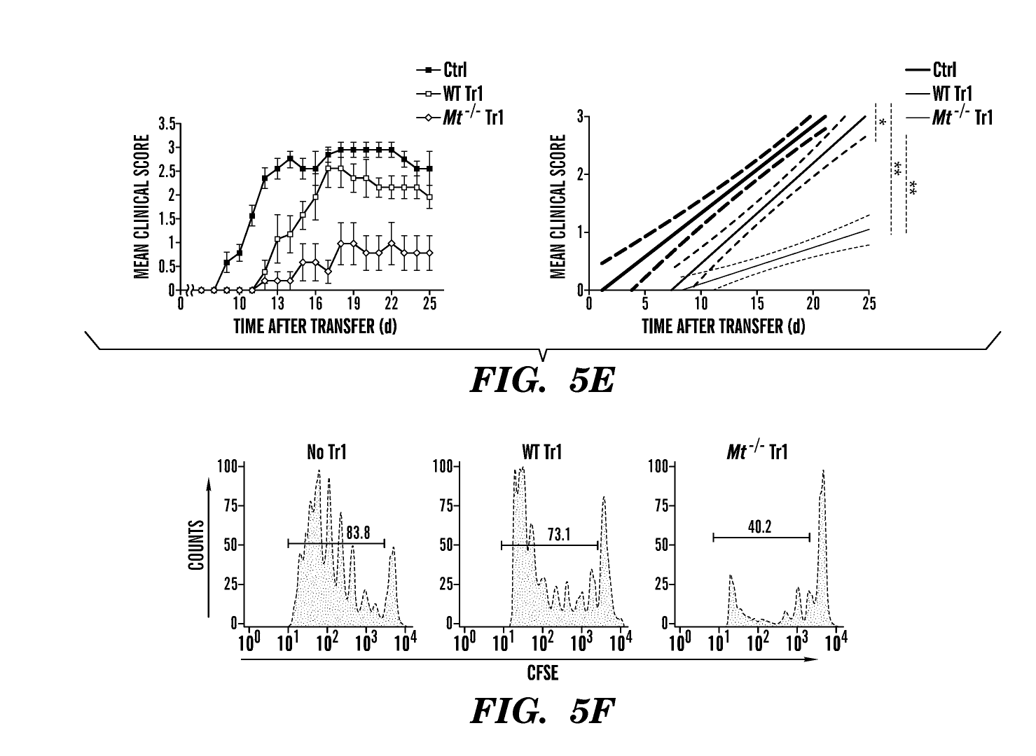

In order to further understand the role of MTs and their relevance to the function of Tr1 cells in vivo, the impact of MT-deficient Tr1 cells in an adoptive transfer model of EAE was assessed. The inventors first immunized WT or Mt^{-/-} mice with MOG₃₅₋₅₅ peptide with CFA. 10 days following immunization lymphocytes were isolated from immunized WT or Mt^{-/-} mice and reactivated them under various conditions. The response was antigen specific as depicted by the proliferation with MOG but not with OVA peptide (FIG. 5A-5D). When these cells were restimulated by MOG₃₅₋₅₅ and IL-23, which has been reported to induce and reactivate Th17 cells (16), IL-17 production from CD4⁺ T cells was not altered (FIG. 5A-5B). Although there was no significant change in IL-10 with specific antigen MOG₃₅₋₅₅ by FACS, there was a higher production of IL-10 in CD4⁺ T cells from Mt^{-/-} mice than WT mice when T cells were reactivated in the presence of MOG₃₅₋₅₅ in the cultured supernatant which was further amplified in the presence IL-27 (FIG. 5C-5D). Moreover, IFN-γ production by cells from WT and Mt^{-/-} mice was comparable following either stimulation condition (FIG. 5A-5D). Consistent with our in vitro data, these results confirm the specific role MTs play in the regulation of IL-10 in IL-27-stimulated Tr1 cells.

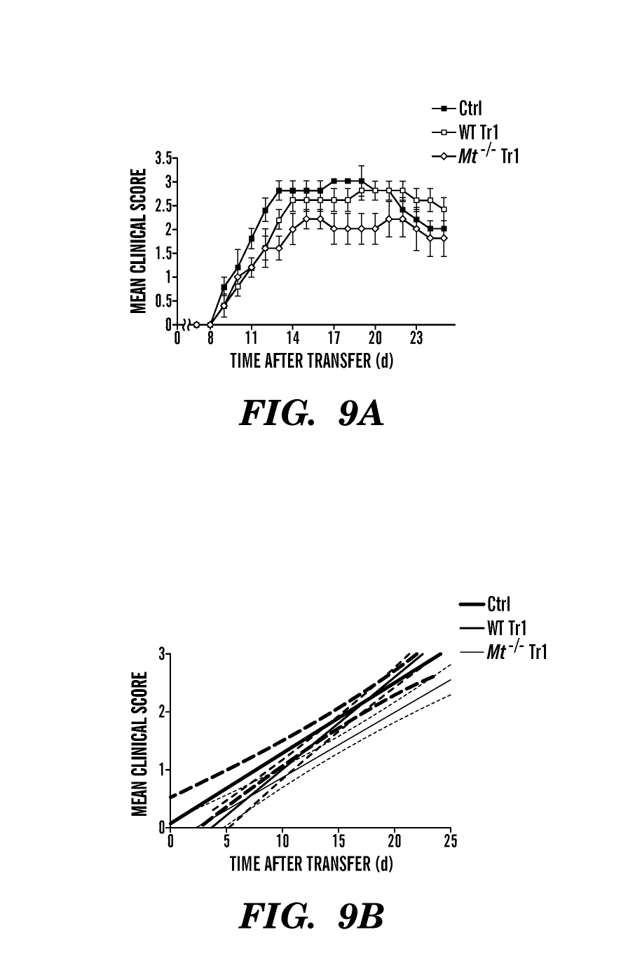

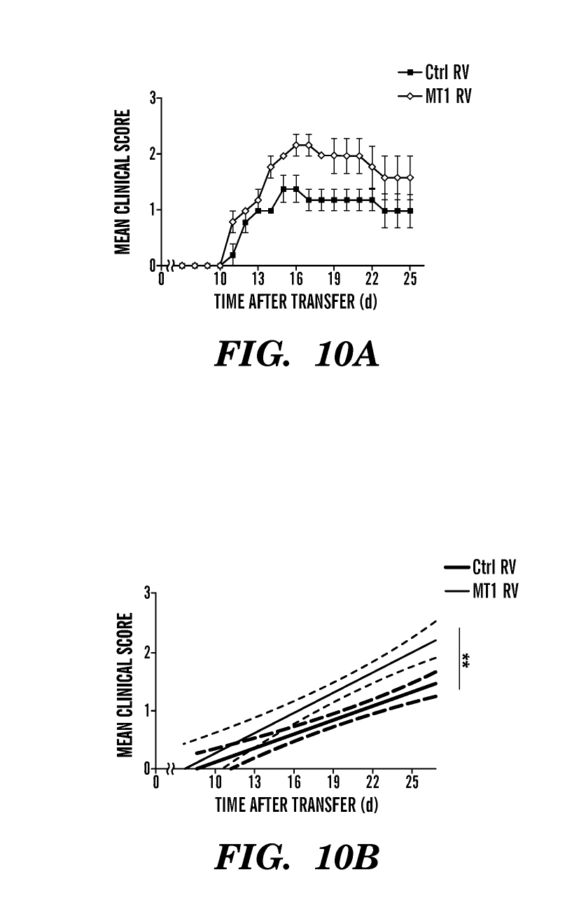

Next, the inventors assessed whether Mt^{-/-} Tr1 cells display enhanced ability to suppress autoimmunity since IL-10 production has been used as a criterion for Tr1 cell anti-inflammatory activity. Pathogenic effector T cells by from MOG immunized WT mice were generated. Simultaneously, WT or Mt^{-/-} T cells were cultured in the presence of MOG and IL-27 to generate antigen specific Tr1 cells. Since Mt^{-/-} mice are on the SV129 background and this strain is not optimal for EAE induction by MOG₃₅₋₅₅ (17), the inventors used an alternative recipient strain (SV129×B6) F1 which shares the compatible genetic background with SV129 while being susceptible to EAE following adoptive transfer (18). The inventors then adoptively transferred WT effector T cells with or without differentiated Tr1 cells from either WT or Mt^{-/-} mice at a ratio of 3:1. As expected, WT Tr1 cells significantly suppressed EAE development. However, Mt^{-/-} Tr1 cells suppressed disease more efficiently than WT Tr1 cells both in terms of disease severity and incidence (FIG. 5E). Using the same adoptive transfer system, WT effector cells were labelled with CFSE prior to transfer. On day 4 after transfer, lymphocytes were isolated from lymph nodes of the recipient mice and CFSE effector cell proliferation was analyzed. There was a significant reduction of effector cells proliferation if they had been transferred together with Tr1 cells. Moreover, Mt^{-/-} Tr1 cells displayed a superior suppressive capacity, inhibiting effector T cell proliferation more profoundly compared to WT Tr1 cells (FIG. 5F). The inventors further performed the titrated Tr1 cell in vivo suppression EAE experiment by titrating Tr1 cell. Mt^{-/-} Tr1 cells exhibited the suppressive capacity on the disease process while WT Tr1 cell suppressive effect was no longer dominant when the ratio of the Teff:Tr1 at 5:1 (FIG. 9).

### Example 5

As one of the suppressive T cell subsets, Tr1 cell has been described to regulate inflammation, graft versus-host disease and autoimmunity by producing IL-10 (3). The results presented in this study shows that the nonenzymatic proteins MT1 and MT2 can negatively regulate the production of IL-10 but not IFN-γ during Tr1 cells development and thus regulates the anti-inflammatory properties of those cells. Loss of MT1 and MT2 enhanced IL-10 in Tr1 cell upon IL-27 or vitamin D3/dexamethasone stimulation without affecting IFN-γ. Furthermore, the enhancement of IL-10 increases the suppressive capacity of MT deficient Tr1 cells to suppress effector CD8+ T-cells, in turn leading to the abatement of autoimmunity. Our results reveal that MTs blunts IL-10 secretion from Tr1 cells by preventing the activation of the transcription factor STAT1 and STAT3. Consistent with this data, anti-CD3-induced generation of Tr1 cells was increased in the absence of MTs in vivo, underscoring the key role of MTs in regulating IL-10 production in Tr1 cells.

While the MTs key roles in mediating heavy metal detoxification have been abundantly documented (19), their implication in the control of gene transcription is still unclear. It has been proposed that MT could bind to the p50 subunit of the NF-kB complex, thereby increasing its ability to act as a transcriptional activator (20), while so far there are no evidences shown that MTs exhibit any DNA-binding sites, making them unlikely to directly regulate gene expression. However, it has been shown that MTs can control the binding of the estrogen receptor to its DNA-binding site by modulating zinc levels (21). Thus, the inventors have demonstrated that MTs regulate induction of pSTAT1 and pSTAT3, both of which are required for induction of many important cytokines-driven functions in T cells. Whether MTs directly bind to the STAT1 or STAT3 and interfere with their phosphorylation or indirectly by interfering with Jak-mediated phosphorylation is not clear at this stage. However, since it was reported that Zinc binding disrupts the association of STAT3 with Jak2 kinase (22), zinc may be involved in the MT-driven inhibition of phosphorylation of STAT1/3. Nonetheless loss of MTs resulted in enhanced phosphorylation and transcription of STAT1 and STAT3, suggesting STAT1/3 but not STAT4 as one of the targets of MTs. During Th17 cell differentiation, MTs expression does not regulate the expression of pro-inflammatory proteins such as IL-17. The role of MTs for IL-10 production within Th17 cells is possible, considering the activation of STAT3 during Th17 differentiation (23). Together with the fact that MTs are also induced in the Th17 cells, it cannot be excluded that MTs also affect other T cells differentiation. Furthermore, as disclosed herein in Example 6, the inventors surprisingly discovered that MT functions differently in CD8+ cells, where MT functions to decrease the activity or proliferation of exhausted CD8+ cells, and an inhibitor of MT can promote the activity and/or proliferation and/or differentiation of exhausted CD8+ cells.

Proinflammatory cytokines such as IL-6 induce MTs by activating the transcription factor STAT1 and STAT3 (11). Endotoxin (LPS) produced during bacterial infection, has also been shown elevate the MT expression level (11). Both LPS and IL-6 can initiate STAT1 and STAT3 expression and activation. STAT1Naïve CD8⁺ T cells from either wild type (WT) or metallothionein deficient mice (MT^{-/-}) mice were cultured in the presence of 2 ug/ml anti-CD3 and irradiated APC. After 48 h, IL-27 (20 ng/ml) was added to cultures. Cells were harvested three days later and gene expression analyzed using Nanostring ncounter technology using a custom codeset. Naïve CD8⁺ T cells from either wild type (WT) or metallothionein deficient mice (MT^{-/-}) mice were cultured in the presence of 2 ug/ml anti-CD3 and irradiated APC. After 48 h, IL-27 (20 ng/ml) was added to cultures. Cells were harvested three days later and gene expression analyzed using Nanostring ncounter technology using a custom codeset and STAT3 in turn can directly bind to the promoter of MTs and potentiate its transcription (11), forming a feedback inhibitory loop whereby STAT1/3 induction of MTs results in decrease induction and/or activation of STAT1/3. This mechanism of action would be reminiscent of the action of the suppressors of cytokine signaling 3 (SOCS3), which is induced by STAT3, and limits STAT3 phosphorylation, thereby dampening the secretion of proinflammatory cytokines like IL-17 (24). Here, the induction of MTs by IL-27 would limit the induction of Tr1 cells to prevent excessive immune regulation that might favor the emergence of viral infections or cancers.

Both STAT1 and STAT3 have been shown to be phosphorylated upon IL-27 signaling (6, 25), leading to transactivation of IL-10. The absence of MTs results in hyper-phosphorylation of both STAT1 and STAT3 under the stimulation of IL-27. On the other hand, either STAT1 or STAT3 deficient Tr1 cells exhibit reduced MT1 and MT2 expression. The inventors next assessed if MTs and STATs compete with each other during Tr1 cell development to control IL-10 production. Although MT1 overexpression can lead to IL-10 suppression from Tr1 cells, co-upregulation of STAT1/3 results in restoration of IL-10. This indicates that STAT1 or STAT3 can override MT dependent suppression of IL-10 during Tr1 differentiation. MTs possibly function at the later stage of Tr1 cell development according to their expression profile. It is conceivable, that in the setting of an infection, STAT1 and STAT3 are upregulated early on and induce Tr1 cells, whereas after the immune system has cleared the pathogen, MTs might play a role in immuno-homeostasis, ensuring the reinstatement of the necessary balance between activating and regulatory responses.

It has been described that MT proteins play significant roles during different inflammatory conditions, such as CIA (26) or EAE (27) and the function of MTs within different cell compartments varies. During CNS inflammation, it has been reported that MTs play an important role for EAE recovery, since MT proteins were found to be elevated within the CNS, specifically in astrocytes and activated macrophages (27). Together with the Zn-MT2 treatment resulting in EAE reduction, this implicates that MTs have a strong neuroprotective effect within the CNS (28). Moreover, in the absence of MTs, mice exhibited enhanced production of proinflammatory cytokines such as IL-1 and TNF-α which in turn can lead to inhibition of leukocyte recruitment and ameliorate EAE (29, 30). Herein, the inventors demonstrate that MTs negatively regulate IL-10 production within Tr1 cells and their pro-inflammatory role in the EAE model. Passive transfer EAE experiments allowed us to dissect the function of MTs specifically in T cells, excluding any effects due to the neuroprotective role of MTs. On the other hand, the inventors and others have previously reported that the repetitive administration of anti-CD3 can induce Tr1 cells in the gut (1, 15). Repetitive administration of anti-CD3 induced Tr1 cells in the gut more efficiently when the inventors utilized Mt^{-/-} BM chimera compared to Mt^{-/-} mice, which further emphasizes the importance of environment to control MTs response. This data further demonstrates that MTs have divergent functions in the immune response, depending on tissue and cells. And it is important to evaluate distinct effects of MTs in specific circumstances to identify all their various and sometimes contradicting functions.

Altogether, herein the inventors have discovered that MT1 and MT2 are negative regulators of Tr1 cell differentiation and IL-10 production. Meanwhile, the inventors also demonstrate the balance between MT and STAT signaling in controlling Tr1 cell development. Thus, the inventors have demonstrated that MTs, beyond their essential role in the regulation of metal homeostasis, also shape the quality of immune responses in vivo. By identifying a specific function for MTs in Tr1 cells, the inventors have identified MTs as a novel target for development of selective therapeutic strategies for autoimmunity.

### Example 6

It has been previously reported that inhibition of MT1 and MT2 increases neurodegeneration in experimental autoimmune encephalomyelitis (EAE) (Penkowa et al., J. Neuroimmunology., 2001; 119; 248-260), and that the treatment with metallothioneins protects neurons and prevents demyelination and axonal damage (Penkowa et al., J. Neurosci Res., 2003; 72: 574-586), which are incorporated herein in its entirety by reference.

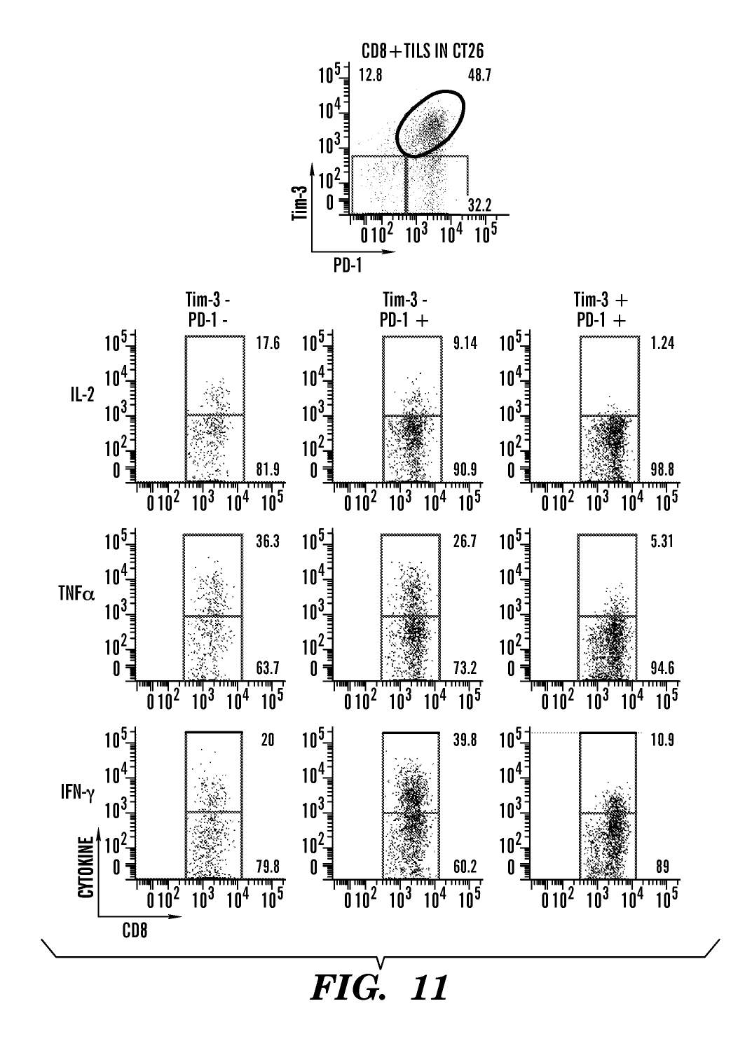

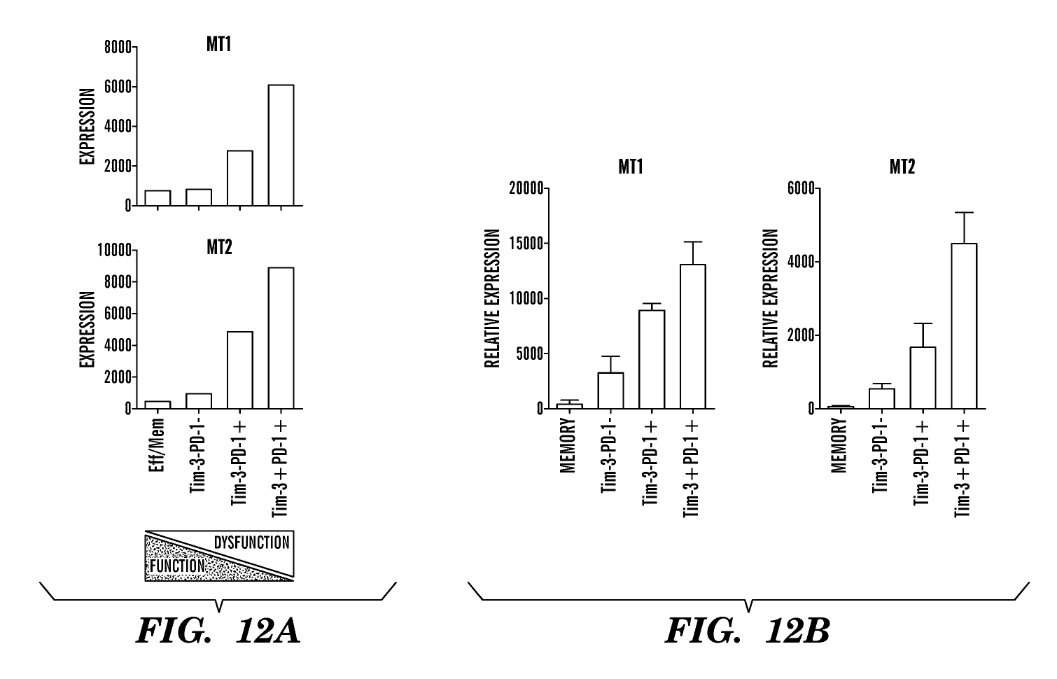

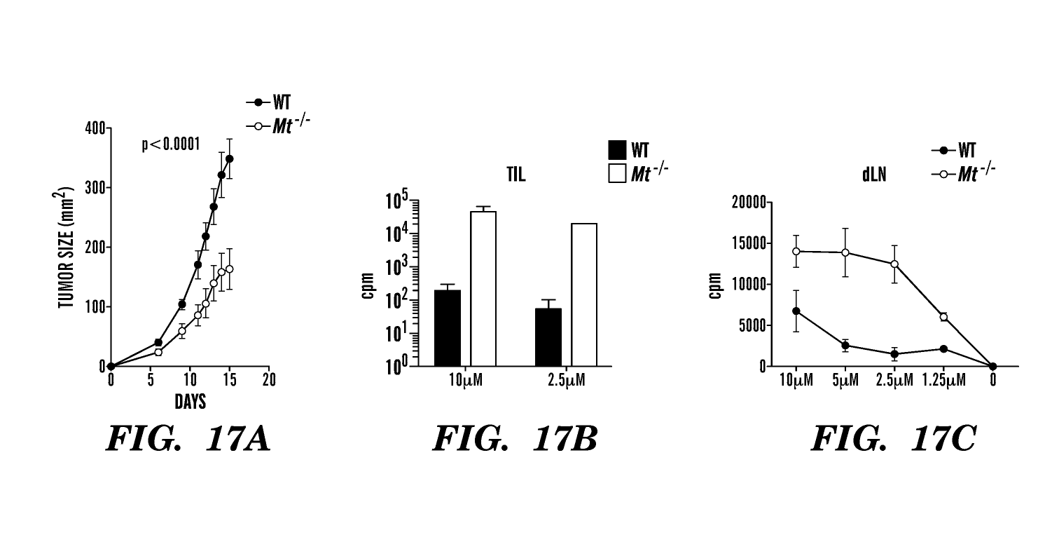

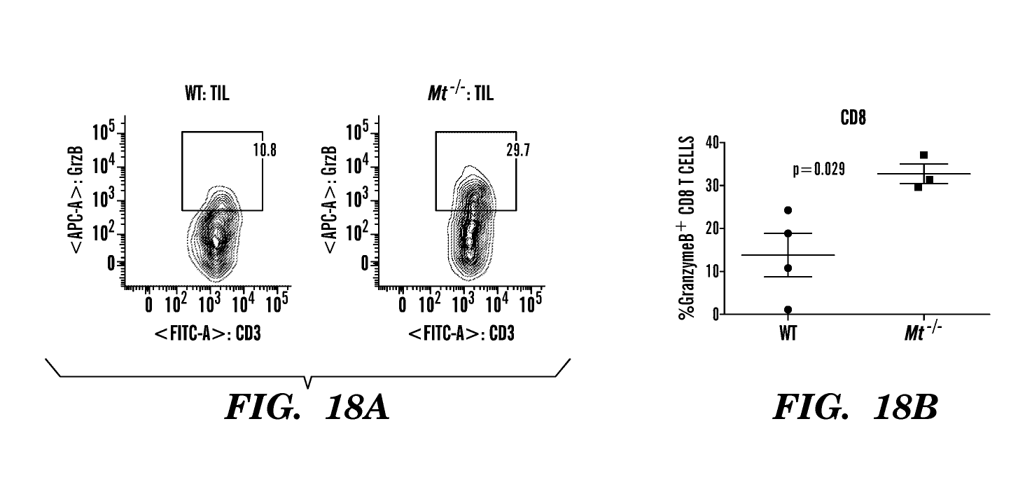

The inventors herein demonstrate herein that Metallothionein 1 and 2 are upregulated in dysfunctional/exhausted CD8+ T cells in cancer. Exhausted CD8+ T cells are defined by impaired function and surface expression of Tim-3 and PD-1 (FIG. 11). The inventors demonstrate that metallothionein 1 and 2 are progressively up-regulated in CD8+ T cells as they develop the exhausted T cell phenotype (FIG. 12) and that metallothionein 1 and 2 deficient mice exhibit delayed melanoma tumor growth and improved response to tumor antigen (FIGS. 17 and 18).

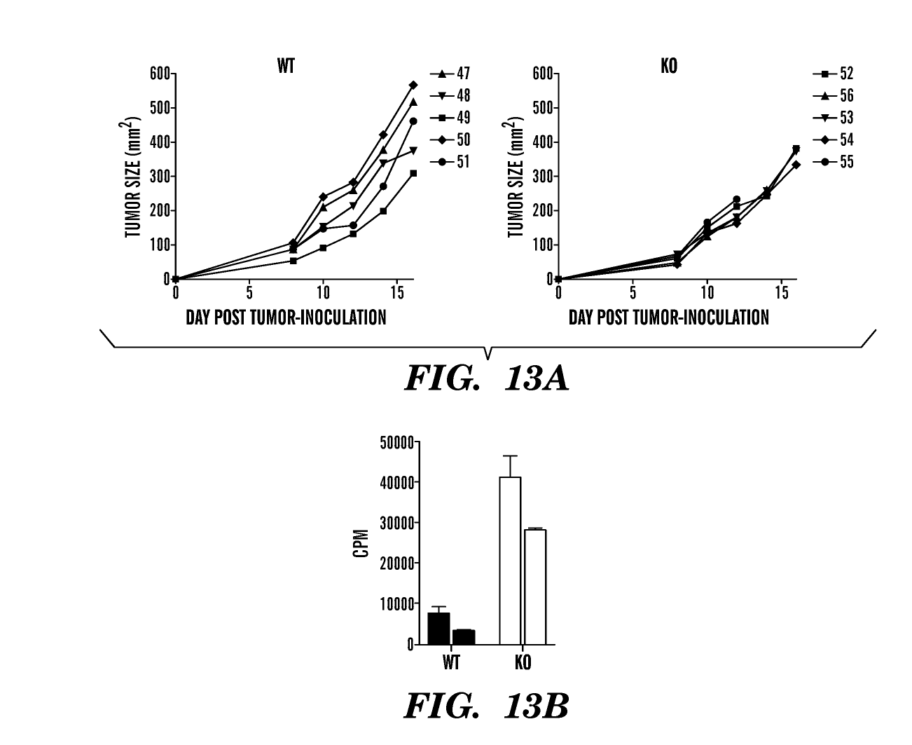

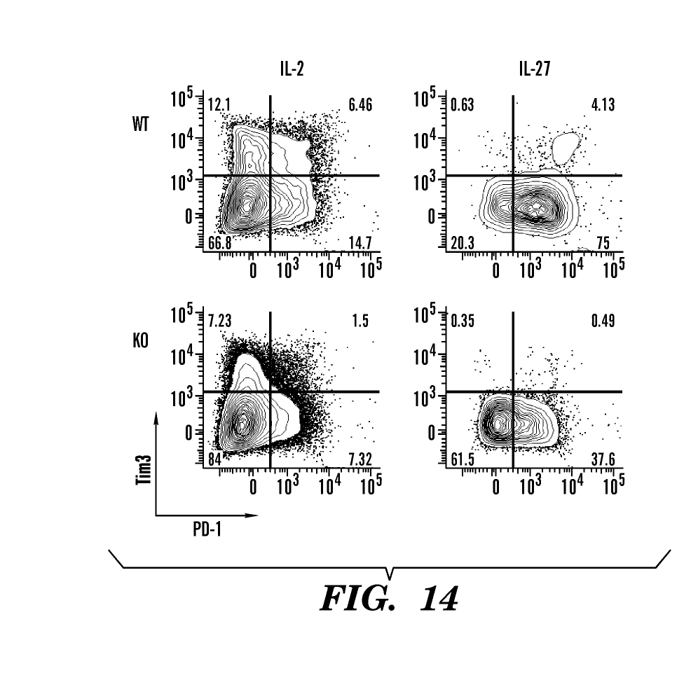

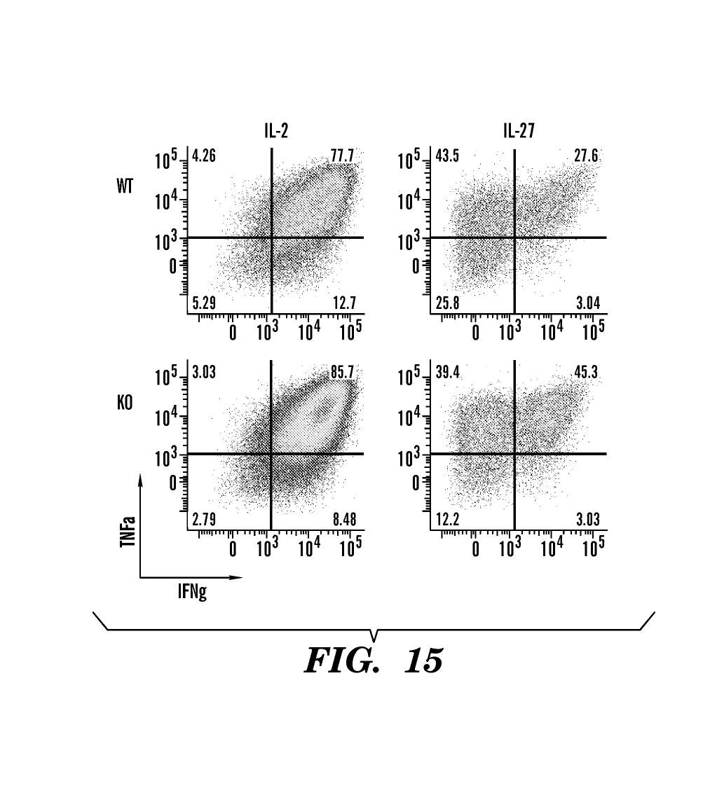

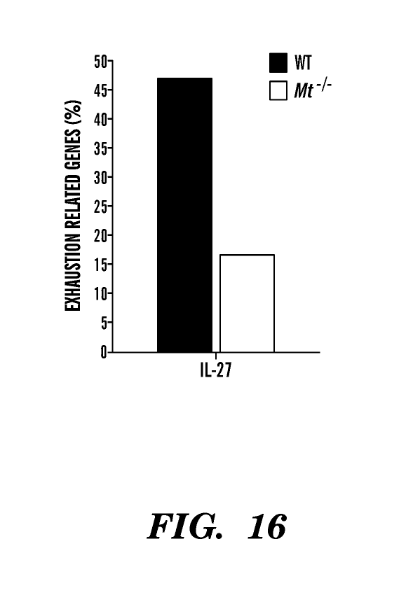

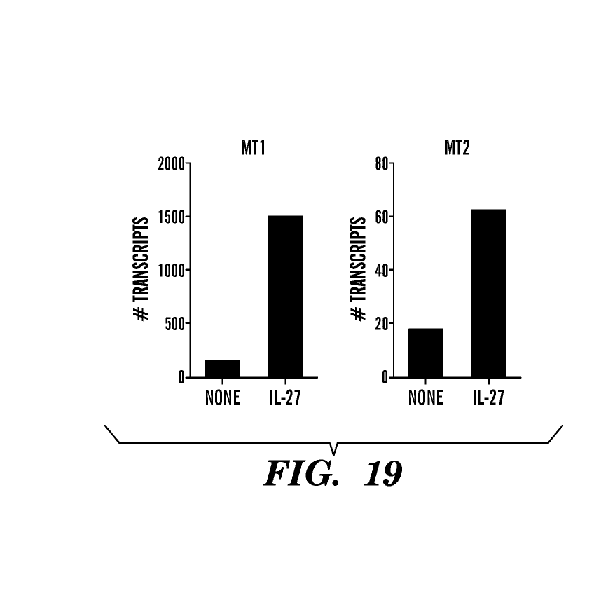

The inventors further show that the effect of metallothionein in improving anti-tumor immunity is T cell intrinsic (FIG. 13) and that IL-27 induces metallothionein 1 and 2 specifically in CD8 T cells (FIG. 19). Metallothionein 1/2 deficient T cells show defective upregulation of Tim-3 and PD-1 in response to IL-27 (FIG. 14). Furthermore, metallothionein MT1 and/or MT2 deficient T cells do not develop dysfunctional/exhausted phenotype in response to IL-27 as indicated by maintenance of polyfunctional T cells (FIG. 15). Moreover, metallothionein 1/2 deficient T cells fail to upregulate genes related to T cell exhaustion in response to IL-27 (FIG. 16). Accordingly, based on the inventors' data, it would follow that activation of MTs, e.g., activation of MT1 and/or MT2, e.g., using an activator of MT1 and/or MT2 in a T-cell decreases anti-tumor T-cell responses. That is, inhibition of MT1 and/or MT2 in such T-cells can be used to treat cancer, while activation of MT1 and/or MT2 in such T cells can be used to treat autoimmune disease or transplant rejection.

### REFERENCES

All references are incorporated herein in their entirety by reference.
1. Apetoh L, et al. (2010) The aryl hydrocarbon receptor interacts with c-Maf to promote the differentiation of type 1 regulatory T cells induced by IL-27. Nat Immunol 11(9):854-861.
2. Fitzgerald D C, et al. (2007) Suppression of autoimmune inflammation of the central nervous system by interleukin 10 secreted by interleukin 27-stimulated T cells. Nat Immunol 8(12):1372-1379.
3. Roncarolo M G, et al. (2006) Interleukin-10-secreting type 1 regulatory T cells in rodents and humans. Immunol Rev 212:28-50.
4. Xie Y, et al. (2009) Tumor apoptotic bodies inhibit CTL responses and antitumor immunity via membrane-bound transforming growth factor-beta1 inducing CD8+ T-cell anergy and CD4+ Tr1 cell responses. Cancer Res 69(19):7756-7766.
5. Awasthi A, et al. (2007) A dominant function for interleukin 27 in generating interleukin 10-producing anti-inflammatory T cells. Nat Immunol 8(12):1380-1389.
6. Stumhofer J S, et al. (2006) Interleukin 27 negatively regulates the development of interleukin 17-producing T helper cells during chronic inflammation of the central nervous system. Nat Immunol 7(9):937-945.
7. Pot C, et al. (2009) Cutting edge: IL-27 induces the transcription factor c-Maf, cytokine IL-21, and the costimulatory receptor ICOS that coordinately act together to promote differentiation of IL-10-producing Tr1 cells. Journal of immunology 183(2):797-801.
8. Pot C, Apetoh L, Awasthi A, & Kuchroo V K (2010) Molecular pathways in the induction of interleukin-27-driven regulatory type 1 cells. J Interferon Cytokine Res 30(6):381-388.
9. Hamer D H (1986) Metallothionein. Annu Rev Biochem 55:913-951.
10. Ghoshal K, et al. (2001) Influenza virus infection induces metallothionein gene expression in the mouse liver and lung by overlapping but distinct molecular mechanisms. Mol Cell Biol 21(24):8301-8317.
11. Lee D K, Carrasco J, Hidalgo J, & Andrews G K (1999) Identification of a signal transducer and activator of transcription (STAT) binding site in the mouse metallothionein-I promoter involved in interleukin-6-induced gene expression. Biochem J 337 (Pt 1):59-65.
12. Banat F J, et al. (2002) In vitro generation of interleukin 10-producing regulatory CD4(+) T cells is induced by immunosuppressive drugs and inhibited by T helper type 1 (Th1)- and Th2-inducing cytokines. The Journal of experimental medicine 195(5):603-616.
13. Wang H, et al. (2011) IL-27 induces the differentiation of Tr1-like cells from human naive CD4+ T cells via the phosphorylation of STAT1 and STAT3. Immunol Lett 136(1):21-28.
14. Saraiva M, et al. (2009) Interleukin-10 production by Th1 cells requires interleukin-12-induced STAT4 transcription factor and ERK MAP kinase activation by high antigen dose. Immunity 31(2):209-219.
15. Kamanaka M, et al. (2006) Expression of interleukin-10 in intestinal lymphocytes detected by an interleukin-10 reporter knockin tiger mouse. Immunity 25(6):941-952.
16. Langrish C L, et al. (2005) IL-23 drives a pathogenic T cell population that induces autoimmune inflammation. The Journal of experimental medicine 201(2):233-240.
17. Stromnes I M & Goverman J M (2006) Active induction of experimental allergic encephalomyelitis. Nature protocols 1(4): 1810-1819.
18. Furlan R, et al. (1999) Caspase-1 regulates the inflammatory process leading to autoimmune demyelination. Journal of immunology 163(5):2403-2409.
19. Lai Y, Yip G W, & Bay B H (2011) Targeting metallothionein for prognosis and treatment of breast cancer. Recent Pat Anticancer Drug Discov 6(2):178-185.
20. Abdel-Mageed A B & Agrawal K C (1998) Activation of nuclear factor kappaB: potential role in metallothionein-mediated mitogenic response. Cancer Res 58(11):2335-2338.
21. Cano-Gauci D F & Sarkar B (1996) Reversible zinc exchange between metallothionein and the estrogen receptor zinc finger. FEBS Lett 386(1):1-4.
22. Kitabayashi C, et al. (2010) Zinc suppresses Th17 development via inhibition of STAT3 activation. Int Immunol 22(5):375-386.
23. Xu J, et al. (2009) c-Maf regulates IL-10 expression during Th17 polarization. Journal of immunology 182(10):6226-6236.
24. Chen Z, et al. (2006) Selective regulatory function of Socs3 in the formation of IL-17-secreting T cells. Proc Natl Acad Sci USA 103(21):8137-8142.
25. Stumhofer J S, et al. (2007) Interleukins 27 and 6 induce STAT3-mediated T cell production of interleukin 10. Nat Immunol 8(12):1363-1371.
26. Youn J, et al. (2002) Metallothionein suppresses collagen-induced arthritis via induction of TGF-beta and down-regulation of proinflammatory mediators. Clin Exp Immunol 129(2):232-239.
27. Espejo C, Penkowa M, Demestre M, Montalban X, & Martinez-Caceres E M (2005) Time-course expression of CNS inflammatory, neurodegenerative tissue repair markers and metallothioneins during experimental autoimmune encephalomyelitis. Neuroscience 132(4):1135-1149.
28. Penkowa M. & Hidalgo J (2003) Treatment with metallothionein prevents demyelination and axonal damage and increases oligodendrocyte precursors and tissue repair during experimental autoimmune encephalomyelitis. J Neurosci Res 72(5):574-586.
29. Penkowa M, Espejo C, Martinez-Caceres E M, Montalban X, & Hidalgo J (2003) Increased demyelination and axonal damage in metallothionein I+II-deficient mice during experimental autoimmune encephalomyelitis. Cell Mol Life Sci 60(1):185-197.
30. Penkowa M, et al. (2001) Altered inflammatory response and increased neurodegeneration in metallothionein I+II deficient mice during experimental autoimmune encephalomyelitis. J Neuroimmunol 119(2):248-260.

1. A method for promoting CD4+ T cell differentiation into type 1 regulatory T (Tr1) CD4+ cells and/or increasing the activity of type 1 regulatory T (Tr1) CD4+ cells in a subject in need thereof, the method comprising administering to the subject a composition comprising an inhibitor of a metallothionein (MT) or an activator agent which increases the phosphorylation of STAT1 and/or STAT3 or increases the phosphorylation of STAT1 and/or STAT3.

2-58. (canceled)

59. The method of claim 1, wherein increasing the activity of Tr1 CD4+ cells is increasing IL-10 secretion from type 1 regulatory T (Tr1) CD4+ cells.

60. The method of claim 1, wherein the metallothionein is an isoform of metallothionein 1 (MT1) or metallothionein 2 (MT2).

61. The method of claim 1, wherein the inhibitor of a metallothionein is selected from the group consisting of: RNAi agent, oligonucleotide, antibody, antibody fragment, peptide inhibitor, protein inhibitor, aptamer, and functional fragments thereof.

62. The method of claim 1, wherein the subject in need thereof has an autoimmune disease and/or is a transplant recipient.

63. The method of claim 62, wherein the autoimmune disease is selected from the group consisting of: myelitis, Addison's disease, Celiac disease (gluten-sensitive enteropathy), Dermatomyositis, Graves disease, Hashimoto's thyroiditis, Multiple sclerosis, Myasthenia gravis, Pernicious anemia; Reactive arthritis, Rheumatoid arthritis, Sjogren syndrome, Systemic lupus erythematosus, inflammatory bowel disease (IBS), graft-versus-host disease and Type I diabetes.

64. The method of claim 63, wherein the myelitis is selected from poliomyelitis (PM), dermatomyositis (DM) or inclusion body myositis (IBM).

65. The method of claim 1, wherein the proliferation of Tr1 CD4+ cells is the proliferation of IL-27-induced proliferation of Tr1 CD4+ cells.

66. The method of claim 1, wherein an inhibitor of MT contacts Tr1 CD4+ cells ex vivo, and wherein the treated Tr1 CD4+ cells are administered to the subject.

67. A pharmaceutical composition comprising an inhibitor of a metallothionein (MT) and a pharmaceutically acceptable carrier for at least one of:
(i) to promote the differentiation of CD4+ cells to type 1 regulatory T (Tr1) CD4+ cells in a subject in need thereof, or
(ii) to increase IL-10 production from type 1 regulatory T (Tr1) CD4+ cells in a subject in need thereof, or
(iii) to increase the activity or proliferation of exhausted CD8+ T-cells in a subject in need thereof, or
(iv) to decrease CD8+ T-cell exhaustion in a subject in need thereof.

68. A method for increasing the differentiation or proliferation of functionally exhausted CD8+ T-cells in a subject in need thereof, or decreasing CD8+ T-cell exhaustion in a subject in need thereof, the method comprising administering to the subject a pharmaceutical composition of claim 67.

69. The method of claim 68, wherein the subject in need thereof has cancer or metastatic cancer.

70. The method of claim 68, wherein the subject has a chronic infection.

71. The method of claim 68, wherein the metallothionein is an isoform of metallothionein 1 (MT1) or metallothionein 2 (MT2).

72. The method of claim 68, wherein the inhibitor of a metallothionein is selected from the group consisting of: RNAi agent, oligonucleotide, antibody, antibody fragment, peptide inhibitor, protein inhibitor, aptamer, and functional fragments thereof.

73. The method of claim 69, wherein the metastatic cancer is selected from the group consisting of: colon cancer, prostate cancer, breast cancer, kidney cancer, leukemia, blood cancer and the like.

74. The method of claim 68, wherein an inhibitor of MT is contacts CD8+ T-cells ex vivo, and wherein the treated CD8+ T-cells are administered to the subject.

75. A pharmaceutical composition comprising an activator of a metallothionein (MT) and a pharmaceutically acceptable carrier for at least one of: (i) to decrease the differentiation of CD4+ cells to type 1 regulatory T (Tr1) CD4+ cells, or (ii) decrease the activity of type 1 regulatory T (Tr1) CD4+ cells in a subject in need thereof.

76. The pharmaceutical composition of claim 75, wherein decreasing the activity of Tr1 CD4+ cells is decreasing IL-10 production from type 1 regulatory T (Tr1) CD4+ cells in a subject in need thereof.

77. The pharmaceutical composition of claim 75, wherein the metallothionein is an isoform of metallothionein 1 (MT1) or metallothionein 2 (MT2).

78. The pharmaceutical composition of claim 75, wherein the activator of a metallothionein is selected from the group consisting of: an antibody, antibody fragment, peptide, protein, small molecule, and functional fragments thereof.

79. The pharmaceutical composition of claim 75, wherein the subject in need thereof is in need of reduced IL-10 production.

80. A method to treat an immune disease or prevent transplant rejection in a subject, the method comprising administering a pharmaceutical composition of claim 67, and optionally, an activator agent which increases the phosphorylation of STAT1 and/or STAT3.

81. The method of claim 80, wherein the activator agent increases the phosphorylation of STAT1 or STAT3, or increases the phosphorylation of STAT1 and STAT3, or promotes an increase in IL-10 secretion from type 1 regulatory T (TR1) CD4+ cells, or promotes CD4+ T cell differentiation into type 1 regulatory (Tr1) CD4+ cells and/or promotes activity of type 1 regulatory (Tr1) CD4+ cells in a subject.

82. A method to treat cancer or chronic infection in a subject in need thereof, comprising administering to the subject in need thereof a pharmaceutical composition of claim 67.

83. The method of claim 82, wherein the inhibitor of metallothionein does at least one of; promotes the differentiation of functionally exhausted CD8+ T-cells, promotes the proliferation of functionally exhausted CD8+ T-cells, or decreases CD8+ T-cell exhaustion in the subject.


SEQUENCE LISTING (92 sequences)

SEQ ID NO: 1 - protein, 61 aa, Homo sapiens
MDPNCSCATGGSCTCTGSCKCKECKCTSCKKSCCSCCPMSCAKCAQGCICKGASEKCSCCA

SEQ ID NO: 2 - DNA, 468 bp, Homo sapiens
accaagccttccacgtgcgccttatagcctctcaacttcttgcttgggatctccaacctcaccgcggctcgaaatggaccccaactgctcctgcgccactggtggctcctgcacctgcactggctcctgcaaatgcaaagagtgcaaatgcacctcctgcaagaagagctgctgctcctgctgccccatgagctgtgccaagtgtgcccagggctgcatctgcaaaggggcatcagagaagtgcagctgctgtgcctgatgtccggacagccctgctcgaagatatagaaagagtgacctgcacaaacttggaattttttttccatacaaccctgacccatttactgtattttttttaatgaaatatgtgaatgataataaaagttgctgacttaaaaaaaaaaaaaaaaaaaaaaaaaaaaaaaaaaaaaaaaaaaaaaaaaaaaaaaaaaaaaaaaaaaaaaaaaa

SEQ ID NO: 3 - protein, 61 aa, Homo sapiens
MDPNCSCTTGGSCACAGSCKCKECKCTSCKKCCCSCCPVGCAKCAQGCVCKGSSEKCRCCA

SEQ ID NO: 4 - DNA, 403 bp, Homo sapiens
cctgccctgacttctcatatcttgcctaggaactccaggcttgtcttggctccaaatggatcccaactgctcctgcaccacaggtggctcctgtgcctgcgccggctcctgcaagtgcaaagagtgcaaatgtacctcctgcaagaagtgctgctgctcttgctgccccgtgggctgtgccaagtgtgcccagggctgtgtctgcaaaggctcatcagagaagtgccgctgctgtgcctgatgttgggagagccctgctcccagacataaatagagcaaccagtactaacctggatttttttttttaactaccctgaccggtttgctacattcttttttctattcaatatgtgaaagacaataaaacacttttgacttgaaaaaaaaaaaaaaaaaaaaaaaa

SEQ ID NO: 5 - protein, 61 aa, Homo sapiens
MDPNCSCATGGSCTCAGSCKCKECKCTSCKKSCCSCCPVGCAKCAQGCVCKGASEKCSCCA

SEQ ID NO: 6 - DNA, 519 bp, Homo sapiens
aggaacgcgggcggtgcggactcagcgggccgggtgcaggcgcggagctgggcctctgcgcccggcccgacctccgtctataaatagagcagccagttgcagggctccattctgctttccaactgcctgactgcttgttcgtctcactggtgtgagctccagcatcccctttgctcgaaatggaccccaactgctcttgcgccactggtggctcctgcacgtgcgccggctcctgcaagtgcaaagagtgcaaatgcacctcctgcaagaagagctgctgttcctgctgccccgtgggctgtgccaagtgtgcccagggctgcgtctgcaaaggggcatcggagaagtgcagctgctgtgcctgatgtgggaacagctcttctcccagatgtaaatagaacaacctgcacaacctggatttttttaaaaatacaacactgagccatttgctgcatttctttttatactaaatatgtgactgacaataaaaacaattttgactttaaaaaaaaaaaaaaa

SEQ ID NO: 7 - protein, 61 aa, Homo sapiens
MDPNCSCAAGVSCTCAGSCKCKECKCTSCKKSCCSCCPVGCSKCAQGCVCKGASEKCSCCD

SEQ ID NO: 8 - DNA, 456 bp, Homo sapiens
gccccctcccctgactatcaaagcagcggccggctgttggggtccaccacgccttccacctgccccactgcttcttcgcttctctcttggaaagtccagtctctcctcggcttgcaatggaccccaactgctcctgcgccgctggtgtctcctgcacctgcgctggttcctgcaagtgcaaagagtgcaaatgcacctcctgcaagaagagctgctgctcctgctgccccgtgggctgtagcaagtgtgcccagggctgtgtttgcaaaggggcgtcagagaagtgcagctgctgcgactgatgccaggacaacctttctcccagatgtaaacagagagacatgtacaaacctggattttttttttataccaccttgacccatttgctacattccttttcctgtgaaatatgtgagtgataattaaacactttagacctgaaaaaaaaaaaaaaaa

SEQ ID NO: 9 - protein, 61 aa, Homo sapiens
MDPNCSCAAGVSCTCASSCKCKECKCTSCKKSCCSCCPVGCAKCAQGCICKGASEKCSCCA

SEQ ID NO: 10 - DNA, 396 bp, Homo sapiens
actccgccttccacgtgcacccactgcctcttcccttctcgcttgggaactctagtctcgcctcgggttgcaatggaccccaactgctcctgtgccgctggtgtctcctgcacctgcgccagctcctgcaagtgcaaagagtgcaaatgcacctcctgcaagaagagctgctgctcctgctgccctgtgggctgtgccaagtgtgcccaaggctgcatctgcaaaggggcatcggagaagtgcagctgctgcgcctgatgtcgggacagccctgctcccaagtacaaatagagtgacccgtaaaatctaggattttttgttttttgctacaatcttgacccctttgctacattcccttttttctgtgaaatatgtgaataataattaaacacttag

SEQ ID NO: 11 - protein, 61 aa, Homo sapiens
MDPNCSCEAGGSCACAGSCKCKKCKCTSCKKSCCSCCPLGCAKCAQGCICKGASEKCSCCA

SEQ ID NO: 12 - DNA, 397 bp, Homo sapiens
accacgccctccacgtgttccactgcctcttctcttctcgcttgggaactccagtctcacctcggcttgcaatggaccccaactgctcctgcgaggctggtggctcctgcgcctgcgccggctcctgcaagtgcaaaaagtgcaaatgcacctcctgcaagaagagctgctgctcctgttgccccctgggctgtgccaagtgtgcccagggctgcatctgcaaaggggcgtcagagaagtgcagctgctgtgcctgatgtcgggacagccctgctgtcagatgaaaacagaatgacacgtaaaatccaggattttttttttctacaactccgactcatttgctacattcctttttttctgtgaaatatgtgaataataattaaacacttagacttga

SEQ ID NO: 13 - DNA, 2005 bp, Homo sapiens
aatgaagaaggtgagcaagaggatggacccggatgcacacggacccgggagaggcgaagctgaggacgactgtgcgggggagggagaatggagggggtggggacagcgcgaccacctgggaatgggaaatggccctcagggtggcaggggcggcgcgtacctctggcagcatagcccctcccgcacgagcgggccccaaaagggcggtccccgggagcccaccaggctgcactcaggggatggtgcgcctgacccgcctgctgcgcacagctcaacccaagaccgcaggcgccgcggattccgcgtgggggtgggtgagaagggtactctgcacccggccccctcttctaactataaattgagcttccggttcctagaatccaccacgcctcccacctgccccactgcttcttctcctctcccttaggaactctagcttcacctcgcttcgtaatggaccccaattgctcctgctccactagtaagggataccgggtttcggggctttagaatatccatttccattccagaggatagaatgtacccggggcaggagggaggtgcatgttagctcttcctaaagtggcctcctttactttgcacttctcgtgtttttccctgtcaagcgccttcattaccacttggaatgttgccatcttcccagcgcccccccactttttttttgtttttttgtttgttttttgttttgttttgttttgttttgagacggagtcttgccctgtcgcccaggctggagtgcagtggcccgatcttggctcactgaaagctccgcctcccgggttcacgccattctcctgtctcagcctgaagagtagctgggactacaggcgcccgtcaccacgcccggctaattttttgtatttttggtagagacagggtttcaccgtgttagccaggatggtctcgatctcctgacctcgtgatccgccctcctcggcctcccaaagtggccctcccatttttgaagcgagaagactgaggctcaaaactgccaggtcacccagatagccagtgggttcccgggctgggacccagtgctctgtccagcattagagctgcctgaggtggatgggaggcagggagcattgcctcttagggattcctgatataaggccttggggagtaaaattaggagggcgcctgcccatcccagcctgtggggaaaaaaggtggagctgggcttccgtgtgcctgagtggaacaaggtaccaggtttcccatgcactgagcaggaggatgctgagggcctgctcctaaccctgcagcacactcactgctcactgcctttttctcttcacctgcacctgcaccagctcctgcaaatgcagagagtgcaaatgcacctcctgcaagacgagtgagtgaggggccttccctgggaatcgcagggagcgggggtgctgggctgagtcagaggagggatctcagattctgcaggcaggagcaggctcatcactagcttcccacatcccctgaaacggattcaggatcagagctggaagaacattagggatggttgattcctacccaaccttcattcttaacaatggggaaactgaggtcacaagaatgtaccagctggccaaccacagacctgattcttgagggcttttctcactgaaatgtatggtcctggggaattgcccttcctttgtccccacagtcccagtcactgcctgtccagtcttctgctctgtcctggctctggtggggctgagcaagtttttcacaggaaggctctcactccaaagatccaccattgtctcacagagcacaccatccggacctaaagctcctctgggtctgggtgcgagcttgagccaggcccactgttggggcagagaggtgcctgttcaagtctgctgtgacctgtcactctccccttcttccccaggctgctgctcctgctgccccgtgggctgtgccaagtgtgcccagggatgtgtttgcaaagggacactgacaagtgcagctgctgctcctg

SEQ ID NO: 14 - DNA, 408 bp, Homo sapiens
cgctccaccacgccgtccacgtgcgccttgcggtctctccatttatcgcttgagatctccagccttaccgcggctcgaaatggaccccaactactcctgcaccactggtggctcctgcacgtgcgccggctcctgcaagtgcaaagagtgcaaatgcacctcctgcaagaagagctgctgcttctgctgccccatgggctgagccaagtgtgcccacggctgcatctgcaaagggacgtcggagaagtgcagctgctgtgcctgatgtgggaacagctcttctcccagatgttaatagaacaacctgcacaacctggattttttttttatacaatactgagccatttgctgcatttctttttatattaaatatgtgagtgacaataaaacaattttgacttgaatctt

SEQ ID NO: 15 - DNA, 443 bp, Homo sapiens
gtcccatctccgcctgcaaaaggagcagctggctccaggctccaacgtgccttccagctgcctgactgcctcttcgcctctcccgtcatttcttggctcgaaatggaccccaactgctcctgcgccactgggggctcctgctcctgtgccagctcctgcaagtgcaaagagtgcaaatgaacctcctgcaagaagagctgctgctcctgctgccccatgggctgtgccaagtgtgcccagggctgcgtctgcaaaggggcgtcggagaagtgcagctgctgtgcctgatgtggggacagccctgctcccagatgtaaacagagcaacctgcacaaacctggatttttttttcatacaaccctgagcatttgctacattcctttttctattaaatatgtaaacgacaataaaacagttttgacttgaaaaaaaaaaaaaaaaaa

SEQ ID NO: 16 - protein, 61 aa, Homo sapiens
MDPNCSCTTGVSCACTGSCKCKECKCTSCKKSCCSCCPVGCAKCAHGCVCKGTLENCSCCA

SEQ ID NO: 17 - DNA, 464 bp, Homo sapiens
ccctgagtagaaaagcagccgcaggctgtggcgctccaccacgccgtccgggtgggcctagcagtcgctccatttatcgcttgagatctccagccttaccgcggctcgaaatggaccccaactgctcctgcaccactggtgtctcctgcgcctgcaccggctcctgcaagtgcaaagagtgcaaatgcacctcctgcaagaagagctgctgctcctgctgccccgtgggctgtgccaagtgtgcccacggctgtgtctgcaaagggacgttggagaactgcagctgctgtgcctgatgtgggaacagctcttctcccagatgttaatagaacaagctgcacaacctggattttttttcaatacgatactgagccatttgctgcatttctttttatattaaatatgtgagtgacaataaaacaattttgacttgaatcttaaaaaaaaaaaaaaaaaaaaaaaaa

SEQ ID NO: 18 - protein, 61 aa, Homo sapiens
MDPNCSCSPVGSCACAGSCKCKECKCTSCKKSCCSCCPVGCAKCAQGCICKGTSDKCSCCA

SEQ ID NO: 19 - DNA, 468 bp, Homo sapiens
accacgcttttcatctgtcccgctgcgtgttttcctcttgatcgggaactcctgcttctccttgcctcgaaatggaccccaactgctcctgctcgcctgttggctcctgtgcctgtgccggctcctgcaaatgcaaagagtgcaaatgcacctcctgcaagaagagctgctgctcctgctgccctgtgggctgtgccaagtgtgcccagggctgcatctgcaaagggacgtcagacaagtgcagctgctgtgcctgatgccaggacagctgtgctctcagatgtaaatagagcaacctatataaacctggattttttttttttttttttttgtacaaccctgacccgtttgctacatctttttttctatgaaatatgtgaatggcaataaattcatctagactaaaaaaaaaaaaaaaaaaaaaaaaaaaaaaaaaaaaaaaaaaaaaaaaaaaaaaaaaaaaaaaaa

SEQ ID NO: 20 - DNA, 2025 bp, Homo sapiens
tcaccttcccaggagtggggaatggaagggagggaggcagggccgtgtgtctgctctgttgctgtgaacaagacaaacgtggccaaaggacctggctggcaatgcaggtgaggcaaaggtcacatgggaggcttgtcctctgcacacaactccctcgctgcacttcccgcctcagtcctatctagggacatgagcattaggccggggtagcacacagactcccacagaggcgaagctcaggtcattggtgcggggtgctgggctggggacaccgtgaccacctaaggcttggttcctggaccgctcaggggcaggagtggactccaatccccgggaaattctgtagggctgggattatagcagggacgccgcctaccgccaaagcgagcaggagtcaaagggcagcccccagggtgcgcactggcagcgctagggaggatcctgcggcgggcccgtctgctgcgcacagcccggccctgaaccctagggcgcctctgacactgcgggagcgggggccagacttggggcggggcctctgcgcccggcaccccaccctaactacaaagccagaggtggctcctgagctctgttccccctcccaccggcttgcctgcttcttcgcctctggcttaggtactccagcctcacctggtctccagatggacctcaactgctcctgcgacactgataaaggatgccgggtttcggggccttgggatctccattttcatgccagaggatagaatgtccctgcggcaggaggcaggtgcattttgagccctacctgaagtggattattttactttgtacttctggtgctttttctcttaccaatcgcctttattatcatcactgagaacattaccatagtcccagtgcctcccatttctaaggcgagaggaatgaggctcaaaactgcccctgctccgggtcacctataggccagaggtgtgctgagctgggacctagtgctgtgtccagcttttgagctgcctgggctagtagggatgtgggaaacattgcttcttcaagatttcccactgaggtccccgtgtgctatgaaaaagagggcgcctgccgatctgagcctttgtggagaaaaggtggggctgggcttccatgtgcctgagtggaacaaggtaccaggtttcccatgcactgagcaagaggatgctgagggcctgctcctaaccctgcagcacactcaccgctcactgcctttttctctttcctgcagctgggtcctgcacttatgccagcttctgcaaatgcaaagagtacaaatgcacctcttgcaagaagagtgagtgtggggccttccctgggaatcagggtgctgggcagagtcagaggagggaaccgagagctcagcaggcaggagcaggacagtgacaagcttcccacatcccctttctggtaagtgattctggatcacagctggaaaaacacaagatagttgattcccaactttcattcttaaaatggggaaactgagggcatgtgcgtgcaccaacctgccaagcacagacctgacacttcaggactttcctcacttaagtgtacggttttggggaactggctttcctttgtccctgtagccccagtcactgcctctccagtcttctgtcatctctcaggtataggttgtgctgggcagctttttcactggaagaccctcaccccaaagatccaccagttgagtcctaacagagcaatgccaccctgaactgagggtcctctggggctggaggcagggcttgagccaggcctctgtcagggcagagaggtctccggtcgagtctgctctgacctctcactctccccttcttcccagactgctgctcctgctaccctgtgggctgtgccaagtgtgcccaaggctgcatttgcaaaggggcatcagataagtgcagctgctgtgcctgatgctgggacagccctgctcacagatgtagaaagagtgacctatataaacttggaatttttttccatgcaaccatgacccatttactgtatttgtctctc

SEQ ID NO: 21 - protein, 49 aa, Homo sapiens
MDLSCSCATGGSCTCASSCKCKEYKCTSCKKNCCSCCPMGCAKCAQGCT

SEQ ID NO: 22 - DNA, 376 bp, Homo sapiens
cctgtggcttaggaactccagcctcacctgatctccgaatggacctcagctgctcctgcgccactggtggctcctgcacctgtgccagctcctgcaaatgcaaagagtacaaatgcacctcctgcaagaagaactgctgctcctgctgccccatgggctgtgccaaatgtgcccagggctgcacctgaaaaggggcattggagaagtgcagctgccgtgcctgatgttgggacagccctgcgtccagttgtaaataacgcaacccctacaaatctagtttttttttaaacacaaacctaacccatttactatgtctgttttcttaaatgaaatatgggaatgataataaacattgttggccttaaaaaaaaaaaaa

SEQ ID NO: 23 - DNA, 316 bp, Homo sapiens
cctcttctcttctcgcttgggaacgccggtctcacctcggcttgcaatggaccccaactgctcctgcgccgctggaggctcctacgcctgcgccggctcctgcaagtgcaaaaagtgcaaatgcacctcctgcaagaagagctgctgctcctgttgccccctgggctgtgccaagtgtgcccagggctgcatccgcaaaggggcttcggaaaagtgcagctgctgtgcctgatgtcgggactgccctgctctcggatgaaaacagaatgacacgtaaagtccgggatttttttttctacaactccgactcatttgc

SEQ ID NO: 24 - DNA, 599 bp, Homo sapiens
gccctccatgtccaagggttccacatacacggattcaacaaacctcagatcaaaattattcaaaaaatgaaaataaaaaataataatgcaaccatatgacaccttccagttgcctgactgcctcttcacctttcccatgggaactccagcgtctcctttgctcaaaacggaccccaacagctcctgcaccactggcggctgctggccctgtgctggctcctgcaaatgcaaagagtgcaaatgcacctcctgcaagaagatctgctcctgctgccctgtgggctgtgccaagtgtgcccagggctgcatctgcaaaggggcaacagagaagtgcagctgctgtgcctgatgtggggacagccctgctcccagatgtaaatgaagcaacttggatacaaacctggattttttttttcatatgaccctgagccatttgctacattcctttttctataaatatgtaaataacagtaaaacacttttgacttaaaaaaaaatgcaacaatatgaagtaatacaaataaaaatacagtgttaacaactatatacatagcatttacattgttctaggtattataagtcatctagagatgatttaa

SEQ ID NO: 25 - DNA, 377 bp, Homo sapiens
ctcaggtcaaaaagtttggtaattggtgaacacgccgtccacgtgcgccttgctgtctctccattatcgcttgagatctccagccttaccgcggcttgaaatggaccccaactgctcctgcaccactggtggctcctgcacgtgcgccggctcctgcaagtgcaaagagtgcaaatgcacctcctgcaagaagagctgctgctcctgctgccccatgggctgtgccaagtgtgcccagggctgtgtctgcaaaggggcgtgcagctgctgtgtctgatgtggggacagctcttctcccagatgttaatagaacaacctgcacaacctggttttttttccttaatacaaccctgagccatttgctgcatttcttttca

SEQ ID NO: 26 - DNA, 183 bp, Homo sapiens
atggaccccaactgctcctgctcgcctgatggttcctgcgcctgtgccggctcctgcaaatgaaaagagtgcacatgcacctcctgcaagaagagctgctgctcctgctgccctgtgggctgtgccaagtgtgcccagggctgcatctgcaaagggacgtcggacaactgcagctgctgtgcc

SEQ ID NO: 27 - protein, 60 aa, Homo sapiens
MDPNCSCAAGDSCTCAGSCKCKECKCTSCKKSCCSCCPVGCAKCAQGCICKGASDKCSCC

SEQ ID NO: 28 - DNA, 466 bp, Homo sapiens
cttgccgcgctgcactccaccacgcctcctccaagtcccagcgaacccgcgtgcaacctgtcccgactctagccgcctcttcagctcgccatggatcccaactgctcctgcgccgccggtgactcctgcacctgcgccggctcctgcaaatgcaaagagtgcaaatgcacctcctgcaagaaaagctgctgctcctgctgccctgtgggctgtgccaagtgtgcccagggctgcatctgcaaaggggcgtcggacaagtgcagctgctgcgcctgatgctgggacagccccgctcccagatgtaaagaacgcgacttccacaaacctggattttttatgtacaaccctgaccgtgaccgtttgctatattcctttttctatgaaataatgtgaatgataataaaacagctttgacttgaaaaaaaaaaaaaaaaaaaaaaaaaaaaaaaaaaaaaaaaaaaaaaaa

SEQ ID NO: 29 - protein, 14 aa, Artificial Sequence
  Description of Artificial Sequence Synthetic peptide
KKSSCSCSPVGSAK

SEQ ID NO: 30 - protein, 14 aa, Artificial Sequence
  Description of Artificial Sequence Synthetic peptide
AQGSISKGASDKSS

SEQ ID NO: 31 - protein, 14 aa, Artificial Sequence
  Description of Artificial Sequence Synthetic peptide
MDPNSSSAAGDSST

SEQ ID NO: 32 - protein, 14 aa, Artificial Sequence
  Description of Artificial Sequence Synthetic peptide
SAGSSKSKESKSTS

SEQ ID NO: 33 - protein, 14 aa, Artificial Sequence
  Description of Artificial Sequence Synthetic peptide
AQGSICKGASDKSS

SEQ ID NO: 34 - protein, 14 aa, Artificial Sequence
  Description of Artificial Sequence Synthetic peptide
MDPNCSCAAGDSST

SEQ ID NO: 35 - protein, 14 aa, Artificial Sequence
  Description of Artificial Sequence Synthetic peptide
SAGSCKCKESKSTS

SEQ ID NO: 36 - protein, 12 aa, Artificial Sequence
  Description of Artificial Sequence Synthetic peptide
KGGEAAEAEAEK

SEQ ID NO: 37 - protein, 14 aa, Artificial Sequence
  Description of Artificial Sequence Synthetic peptide
KKSCCSCCPMSCAK

SEQ ID NO: 38 - protein, 14 aa, Artificial Sequence
  Description of Artificial Sequence Synthetic peptide
KKCCGSCCPVGCAK

SEQ ID NO: 39 - protein, 14 aa, Artificial Sequence
  Description of Artificial Sequence Synthetic peptide
KKSCCSCCPVGCAK

SEQ ID NO: 40 - protein, 14 aa, Artificial Sequence
  Description of Artificial Sequence Synthetic peptide
KKSCCSCCPVGCSK

SEQ ID NO: 41 - protein, 14 aa, Artificial Sequence
  Description of Artificial Sequence Synthetic peptide
KKSCCSCCPVGCAK

SEQ ID NO: 42 - protein, 14 aa, Artificial Sequence
  Description of Artificial Sequence Synthetic peptide
KKSCCSCCPLGCAK

SEQ ID NO: 43 - protein, 14 aa, Artificial Sequence
  Description of Artificial Sequence Synthetic peptide
KKSCCSCCPVGCAK

SEQ ID NO: 44 - protein, 14 aa, Artificial Sequence
  Description of Artificial Sequence Synthetic peptide
KKSCCSCCPVGCAK

SEQ ID NO: 45 - protein, 14 aa, Artificial Sequence
  Description of Artificial Sequence Synthetic peptide
KKSCCSCCPVGCAK

SEQ ID NO: 46 - protein, 14 aa, Artificial Sequence
  Description of Artificial Sequence Synthetic peptide
KKSCCSCCPMGCAK

SEQ ID NO: 47 - protein, 14 aa, Artificial Sequence
  Description of Artificial Sequence Synthetic peptide
KKSCCSCCPVGCAK

SEQ ID NO: 48 - protein, 14 aa, Artificial Sequence
  Description of Artificial Sequence Synthetic peptide
KKSCCSCCPVGCAK

SEQ ID NO: 49 - protein, 14 aa, Artificial Sequence
  Description of Artificial Sequence Synthetic peptide
KKSCCSCCPAECEK

SEQ ID NO: 50 - protein, 14 aa, Artificial Sequence
  Description of Artificial Sequence Synthetic peptide
RKSCCPCCPPGCAK

SEQ ID NO: 51 - protein, 14 aa, Artificial Sequence
  Description of Artificial Sequence Synthetic peptide
AQGCICKGASEKCS

SEQ ID NO: 52 - protein, 14 aa, Artificial Sequence
  Description of Artificial Sequence Synthetic peptide
AQGCVCKGSSEKCS

SEQ ID NO: 53 - protein, 14 aa, Artificial Sequence
  Description of Artificial Sequence Synthetic peptide
AQGCVCKGASEKCS

SEQ ID NO: 54 - protein, 14 aa, Artificial Sequence
  Description of Artificial Sequence Synthetic peptide
AQGCVCKGASEKCS

SEQ ID NO: 55 - protein, 14 aa, Artificial Sequence
  Description of Artificial Sequence Synthetic peptide
AQGCICKGASEKCS

SEQ ID NO: 56 - protein, 14 aa, Artificial Sequence
  Description of Artificial Sequence Synthetic peptide
AQGCICKGASEKCS

SEQ ID NO: 57 - protein, 14 aa, Artificial Sequence
  Description of Artificial Sequence Synthetic peptide
AQGCICKGASEKCS

SEQ ID NO: 58 - protein, 14 aa, Artificial Sequence
  Description of Artificial Sequence Synthetic peptide
AQGCICKGASEKCS

SEQ ID NO: 59 - protein, 14 aa, Artificial Sequence
  Description of Artificial Sequence Synthetic peptide
AQGCICKGTSDKCS

SEQ ID NO: 60 - protein, 14 aa, Artificial Sequence
  Description of Artificial Sequence Synthetic peptide
AQGCVCKGASEKCS

SEQ ID NO: 61 - protein, 14 aa, Artificial Sequence
  Description of Artificial Sequence Synthetic peptide
AQGCICKGTSDKCS

SEQ ID NO: 62 - protein, 14 aa, Artificial Sequence
  Description of Artificial Sequence Synthetic peptide
AQGCICKGASDKCS

SEQ ID NO: 63 - protein, 20 aa, Artificial Sequence
  Description of Artificial Sequence Synthetic peptide
AKDCVCKGGEAAEAEAEKCS

SEQ ID NO: 64 - protein, 14 aa, Artificial Sequence
  Description of Artificial Sequence Synthetic peptide
ARGCICKGGSDKCS

SEQ ID NO: 65 - protein, 14 aa, Artificial Sequence
  Description of Artificial Sequence Synthetic peptide
MDPNCSCATGGSCT

SEQ ID NO: 66 - protein, 14 aa, Artificial Sequence
  Description of Artificial Sequence Synthetic peptide
MDPNCSCTTGGSCA

SEQ ID NO: 67 - protein, 14 aa, Artificial Sequence
  Description of Artificial Sequence Synthetic peptide
MDPNCSCATGGSCT

SEQ ID NO: 68 - protein, 14 aa, Artificial Sequence
  Description of Artificial Sequence Synthetic peptide
MDPNCSCAAGVSCT

SEQ ID NO: 69 - protein, 14 aa, Artificial Sequence
  Description of Artificial Sequence Synthetic peptide
MDPNCSCAAGVSCT

SEQ ID NO: 70 - protein, 14 aa, Artificial Sequence
  Description of Artificial Sequence Synthetic peptide
MDPNCSCEAGGSCA

SEQ ID NO: 71 - protein, 14 aa, Artificial Sequence
  Description of Artificial Sequence Synthetic peptide
MDPNCSCAAGVSCT

SEQ ID NO: 72 - protein, 15 aa, Artificial Sequence
  Description of Artificial Sequence Synthetic peptide
MDPNCSCAAAGVSCT

SEQ ID NO: 73 - protein, 14 aa, Artificial Sequence
  Description of Artificial Sequence Synthetic peptide
MDPNCSCSPVGSCA

SEQ ID NO: 74 - protein, 14 aa, Artificial Sequence
  Description of Artificial Sequence Synthetic peptide
MDPNCSCATGGSCS

SEQ ID NO: 75 - protein, 14 aa, Artificial Sequence
  Description of Artificial Sequence Synthetic peptide
MDPNCSCDPVGSCA

SEQ ID NO: 76 - protein, 14 aa, Artificial Sequence
  Description of Artificial Sequence Synthetic peptide
MDPNCSCAAGDSCT

SEQ ID NO: 77 - protein, 15 aa, Artificial Sequence
  Description of Artificial Sequence Synthetic peptide
MDPETCPCPSGGSCT

SEQ ID NO: 78 - protein, 15 aa, Artificial Sequence
  Description of Artificial Sequence Synthetic peptide
MDPRECVCMSGGICM

SEQ ID NO: 79 - protein, 14 aa, Artificial Sequence
  Description of Artificial Sequence Synthetic peptide
CTGSCKCKECKCNS

SEQ ID NO: 80 - protein, 14 aa, Artificial Sequence
  Description of Artificial Sequence Synthetic peptide
CAGSCKCKECKCTS

SEQ ID NO: 81 - protein, 14 aa, Artificial Sequence
  Description of Artificial Sequence Synthetic peptide
CAGSCKCKECKCTS

SEQ ID NO: 82 - protein, 14 aa, Artificial Sequence
  Description of Artificial Sequence Synthetic peptide
CAGSCKCKECKCTS

SEQ ID NO: 83 - protein, 14 aa, Artificial Sequence
  Description of Artificial Sequence Synthetic peptide
CASSCKCKECKCTS

SEQ ID NO: 84 - protein, 14 aa, Artificial Sequence
  Description of Artificial Sequence Synthetic peptide
CAGSCKCKKCKCTS

SEQ ID NO: 85 - protein, 14 aa, Artificial Sequence
  Description of Artificial Sequence Synthetic peptide
CAGSCKCKECKCTS

SEQ ID NO: 86 - protein, 14 aa, Artificial Sequence
  Description of Artificial Sequence Synthetic peptide
CASSCKCKECKCTS

SEQ ID NO: 87 - protein, 14 aa, Artificial Sequence
  Description of Artificial Sequence Synthetic peptide
CAGSCKCKECKCTS

SEQ ID NO: 88 - protein, 14 aa, Artificial Sequence
  Description of Artificial Sequence Synthetic peptide
CASSCKCKECKCTS

SEQ ID NO: 89 - protein, 14 aa, Artificial Sequence
  Description of Artificial Sequence Synthetic peptide
CAGSCKCKECKCTS

SEQ ID NO: 90 - protein, 14 aa, Artificial Sequence
  Description of Artificial Sequence Synthetic peptide
CAGSCKCKECKCTS

SEQ ID NO: 91 - protein, 14 aa, Artificial Sequence
  Description of Artificial Sequence Synthetic peptide
CADSCKCEGCKCTS

SEQ ID NO: 92 - protein, 14 aa, Artificial Sequence
  Description of Artificial Sequence Synthetic peptide
CGDNCKCTTCNCKT